(19) United States
(12) Patent Application Publication
(10) Pub. No.: US 2008/0300251 A1
(43) Pub. Date: Dec. 4, 2008

(54) Derivatives of 3-Azabicyclo[3.1.0] Hexane as Dipeptidyl Peptidase-IV Inhibitors

(76) Inventors: Jitendra A. Sattigeri, Gurgaon (IN); Murugaiah M.S. Andappan, Pudukottai (IN); Kaushal Kishore, Nalanda (IN); Sachin Sethi, Yamuna Nagar (IN); Sachin Ramesh Kandalkar, Ahmednagar (IN); Chanchal Kumar Pal, Midnipur (IN); Dipak C. Mahajan, Jalgaon (IN); Shahadat Ahmed, Gurgaon (IN); Santhosh Sadashiv Parkale, Pune (IN); T. Srinivasan, Chennai (IN); Lalima Sharma, Chandigarh (IN); Vinay S. Bansal, New Delhi (IN); Anita Chugh, New Delhi (IN); Joseph Alexanand Davis, Tiruchy (IN)

Correspondence Address: RANBAXY INC., 600 COLLEGE ROAD EAST, SUITE 2100, PRINCETON, NJ, 08540, US

(21) Appl. No.: 12/065,754

(22) PCT Filed: Sep. 1, 2006
(86) PCT No.: PCT/IB2006/002419

§ 371 Date: Jul. 8, 2008

(30) Foreign Application Priority Data
Sep. 5, 2005 (IN) 2375/DEL/2005

(51) Int. Cl. A61K 31/498; C07D 221/02; A61K 31/439; C07D 215/00; A61K 31/4709; C07D 239/24; A61P 25/00; A61P 29/00; A61P 35/00; A61P 37/00; A61P 3/00; A61K 31/506; C07D 217/00; A61K 31/4725; C07D 241/36

(52) U.S. Cl. 514/249; 546/112; 514/299; 546/168; 514/314; 544/242; 514/256; 546/139; 514/307; 544/353

(57) ABSTRACT

The present invention relates to novel 3-azabicyclo[3.1.0]hexane derivatives as dipeptidyl peptidase-IV inhibitors and the processes for the synthesis of the said compounds. This invention also relates to pharmacological compositions containing the compounds of the present invention, and methods of treating diabetes, especially type 2 diabetes, as well as prediabetes, diabetic dyslipidemia, metabolic acidosis, ketosis, satiety disorders, and obesity. These inhibitors can also be used to treat conditions manifested by a variety of metabolic, neurological, anti-inflammatory, and autoimmune disorders like inflammatory disease, multiple sclerosis, rheumatoid arthritis; viral, cancer and gastrointestinal disorders. The compounds of this invention can also be used for treatment of infertility arising due to polycystic ovary syndrome.

### FIELD OF THE INVENTION

The present invention relates to 3-azabicyclo[3.1.0]hexane derivatives as dipeptidyl peptidase-IV inhibitors and the processes for the synthesis of the compounds. This invention also relates to pharmacological compositions containing the compounds of the present invention, and methods of treating diabetes, especially type 2 diabetes, as well as prediabetes, diabetic dyslipidemia, metabolic acidosis, ketosis, satiety disorders, and obesity. These inhibitors can also be used to treat conditions manifested by a variety of metabolic, neurological, anti-inflammatory, and autoimmune disorders like inflammatory disease, multiple sclerosis, rheumatoid arthritis; viral, cancer and gastrointestinal disorders. The compounds of this invention can also be used for treatment of infertility arising due to polycystic ovary syndrome.

### BACKGROUND OF THE INVENTION

Type 2 diabetes mellitus, also known as “non-insulin dependent diabetes mellitus” (NIDDM), afflicts an estimated 6% of the adult population in western society and is expected to continue to grow at a rate of 6% per annum worldwide. Type 2 diabetes is a complex metabolic disorder, characterized by hyperglycemia and hyperinsulinemia. This results from contribution of impaired insulin secretion from β-cells in pancreas and insulin resistance mainly in muscle and liver. The insulin resistant individuals, in addition to being hyperglycemic, exhibit a constellation of closely related clinical indications, which include obesity, hypertension and dyslipidemia. Uncontrolled hyperglycemia can further lead to late stage microvascular and macrovascular complications such as nephropathy, neuropathy, retinopathy and premature atherosclerosis. In fact, 80% of diabetic mortality arises from atherosclerotic cardiovascular disease (ASCVD).

Presently, several pharmacological agents are available as antihyperglycemic agents to mitigate the conditions manifested in NIDDM (Lancet, 2005, 365, 1333-1346). These include insulin secretagogues, which increase insulin secretion from pancreatic cells [e.g., sulphonyl urea's (glimeperide) and non-sulphonyl ureas (repaglinide)], biguanides, which lower hepatic glucose production (e.g., metformin), and α-glucosidase inhibitors, which delay intestinal absorption of carbohydrates [e.g., acarbose] (Lancet, 2005, 365, 1333-1346). The insulin sensitizers like pioglitazone and rosiglitazone (TZDs), which exhibit their effect by PPARγ agonism, control hyperglycaemia by improving peripheral insulin sensitivity without increasing circulating insulin levels. However, all these agents are associated with one or more of side effects like hypoglycaemia, gastrointestinal side effects including abdominal discomfort, bloating, flatulence, hepatotoxicity, weight gain, dilutional anemia and peripheral edema (Endocrine Rev., 2000, 21, 585-618).

Given its prevalence and complexity of NIDDM, there is a growing need for novel strategies and effective therapeutic approaches for treatment of diabetes. The safe and, preferably, orally bioavailable therapeutic agents, that would accelerate glucose clearance by stimulating endogenous insulin secretion in a glucose-dependent manner, free from hypoglycemic episodes and previously mentioned side effects, would represent an important advance in the treatment paradigm of this disease.

One such novel approach appearing on the horizon involves enhancing the levels of incretin (insulin-secreting) hormones, glucagon-like peptide-1 (GLP-1) and glucose-dependent insulinotropic polypeptide (GIP) (Expert Opin. Investig. Drug, 2005, 14, 57-64). These hormones mediate the process of insulin release from pancreatic β-cells in a glucose-dependent manner. GLP-1 (7-36) is a 29 amino acid containing peptide derived by post translational processing of proglucagon in the L-cells of the distal small intestine in response to the food intake. It promotes multiple synergistic antidiabetic actions including stimulation of insulin secretion, inhibition of glucagon, inducement of feeling fullness and delayed gastric emptying. Administration (continuous infusion) of exogenous GLP-1 in diabetic patients has been demonstrated to be efficacious in lowering blood glucose levels by enhancing glucose-mediated insulin secretion, suppressing glucagon secretion and slowing gastric emptying. Additionally, preclinical studies with GLP-1 or Exendin-4 in streptozotocin-injected neonatal rats have implicated the role of GLP-1 in neogenesis and preservation of β-cells (Current Opin. Pharma., 2004, 4, 589-596; Expert Opin. Investig. Drug, 2003, 12, 87-100).

However, these incretin hormones are very short lived in vivo (t_{1/2 GLP-1}=˜2 min, t_{1/2 GIP}=˜7 min) because they are very rapidly cleaved by the enzyme dipeptidyl peptidase-IV (DPP IV, CD26, EC 3.4.14.5) to GLP-1 (9-36) and GIP (3-42), which are the weak antagonists of GLP-1 and GIP receptors respectively (Reg. Peptides, 2005, 128, 125-134). DPP IV is a serine protease with specificity for cleavage of polypeptides with Pro/Ala at the penultimate position from the N-terminus. It is expressed on the surface of epithelial cells of intestine, liver, kidney proximal tubules, prostrate, corpus luteum, lymphocytes and macrophages. It is now proven that DPP IV inhibition leads to an increase of biologically active forms of both GLP-1 and GIP to therapeutically beneficial levels and thus enhances the body's own normal homeostatic mechanism. As the incretins are released by the body, only in response to the food intake, DPP IV inhibition is not expected to increase the level of insulin at inappropriate times, such as in between meals, which can otherwise lead to hypoglycemia. The initial proof of concept for DPP IV-based therapy has been obtained from DPP IV knockout (KO) mice and other preclinical animal models. The DPP IV KO rat and mice have shown normal glucose tolerance and didn't develop diabetic symptoms, even when fed with fat-rich food. Clinical and pre-clinical studies with DPP IV-resistant GLP-1 analogs like Exenatide have provided indirect but valuable additional validation for the DPP IV target. In clinical trials with an early DPP IV inhibitor, viz., NVP DPP 728, significant improvement in mean 24 hours glucose excursion with lower insulin, glucagon and HbAlc levels were observed in the treated patients. The experimental evidence suggests that DPP IV inhibition offers an added benefit in preservation and regeneration of β cells. DPP IV inhibitors may thus be used in disease modifying therapy in type 1 and late-stage type 2 diabetes.

As GLP-1 has been proposed to be one of the physiological regulators of appetite and food intake, the DPP IV inhibitors may also manifest the beneficial effect of delaying gastric emptying observed with GLP-1. This is corroborated by recent Phase II studies, which demonstrate that no body weight gain was observed with DPP IV inhibitors during the treatment period of the patients with diabetes and obesity (Current Opin. Pharma., 2004, 4, 589-596).

The present invention provides inhibitors and methods for treating conditions mediated by DPP IV, like diabetes, especially, type 2 diabetes mellitus, as well as prediabetes, diabetic dyslipidemia, metabolic acidosis, ketosis, satiety disorders, and obesity. These inhibitors can also be used to treat conditions manifested by a variety of metabolic (Expert Opin. Investig. Drug, 2003, 12, 87-100), neurological (Brain Res., 2005, 1048, 177-184), anti-inflammatory, and autoimmune disorders (Clin. Diagnostic Lab. Immunol. 2002, 9, 1253-1259) like inflammatory disease, multiple sclerosis, rheumatoid arthritis (Clin. Immunol. Immunopath., 1996, 80, 31-37); viral (Clin. Immunol., 1999, 91, 283-295), cancer (Cancer Res., 2005, 65 1325-1334), blood disorders (Blood, 2003, 102, 1641-1648) and gastrointestinal disorders. The compounds of this invention can also be used for treatment of infertility arising due to polycystic ovary syndrome.

WO04/009544 discloses 2-cyano-4-fluoropyrrolidine derivative or its salts. WO03/106456 discloses novel compounds possessing dipeptidyl peptidase-IV enzyme inhibitory activity. WO03/074500 discloses new compounds, which contain fluorine atoms and are DPP-IV enzyme inhibitors. WO03/02553 discloses fluoropyrrolidines as dipeptidyl peptidase inhibitors. WO03/037327 discloses N-(substituted)pyrrolidine derivatives as dipeptidyl peptidase-IV inhibitors. WO03/057666 discloses novel inhibitors of dipeptidyl peptidase-IV. WO01/055105 discloses N-(substituted)-2-cyanopyroles and pyrrolines, which are inhibitors of the enzyme DPP-IV. U.S. Pat. No. 6,011,155 discloses N-(substituted glycyl)-2-cyanopyrrolidines, pharmaceutical compositions containing them and their use in inhibiting dipeptidyl peptidase-IV. The compound (2S)-1-[[(3-hydroxy-1-adamantyl)amino]acetyl]-2-cyanopyrrolidine [vildagliptin] has been disclosed as a potent, selective, and orally bioavailable dipeptidyl peptidase-IV inhibitor with antihyperglycemic properties vide reference J. Med. Chem., 2003, 46(13), 2774-2789.

### SUMMARY OF THE INVENTION

Herein are provided 3-azabicyclo[3.1.0]hexane derivatives possessing dipeptidylpeptidase-IV enzyme inhibitory activity. Also provided are processes for synthesizing such compounds.

These compounds can be used in treatment of conditions mediated by DPP IV, like diabetes, especially, type 2 diabetes mellitus as well as pre-diabetes, diabetic dyslipidemia, metabolic acidosis, ketosis, satiety disorders, and obesity. These inhibitors can also be used for treating conditions manifested by a variety of metabolic, neurological, anti-inflammatory, and autoimmune disorders like inflammatory disease, multiple sclerosis, rheumatoid arthritis viral, cancer and gastrointestinal disorders. The compounds of this invention can also be used for treatment of infertility arising due to polycystic ovary syndrome.

Pharmaceutical compositions containing such compounds are provided together with the pharmaceutically acceptable carriers or diluents, which can be used for the treatment of dipeptidyl peptidase-IV mediated pathologies. These pharmaceutical compositions may be administered or coadministered by a wide variety of routes including, for example, oral or parenteral. The composition may also be administered or coadministered in slow release dosage forms.

The racemates, enantiomers, diastereomers, N-oxides, polymorphs, pharmaceutically acceptable salts and pharmaceutically acceptable solvates of these compounds, prodrugs and metabolites, having the same type of activity, are also provided as well as pharmaceutical compositions comprising the compounds, their metabolites, racemates, enantiomers, N-oxides, polymorphs, solvates, prodrugs or pharmaceutically acceptable salts thereof, in combination with a pharmaceutically acceptable carrier and optionally included excipients.

Other aspects are set forth in the accompanying description, which follows and in the part will be apparent from the description or may be learnt by the practice of the invention.

In accordance with one aspect of the invention, are provided compounds having the structure of Formula I

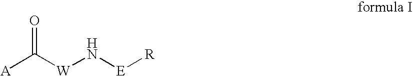

including pharmaceutically acceptable salts, pharmaceutically acceptable solvates, enantiomers, diastereomers, polymorphs, prodrugs, metabolites or N-oxides thereof,
wherein
A can be selected from

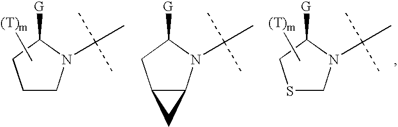

wherein
G can be selected from H, —CN, —COR₁, —CR₂—NOH, —CR₂═NR₂ or B(R₃)(R₄) (wherein R₁ is hydrogen, CF₃, alkyl, aryl or heteroaryl; R₂ is H, alkyl, aryl or heteroaryl; R₃ and R₄ can be independently selected from —OH or —OR₅ [wherein —OR₅ can be hydrolyzed to —OH and R₅ is alkyl, cycloalkyl or aryl]; If R₃ and R₄ are OR₅, then R₃ and R₄ may together form a ring of 5 to 8 atoms), and
T can be cyano, halogen, alkyl, alkenyl, alkynyl, hydroxy, alkoxy, carbonyl, thiocarbonyl, and oxo and n is 0-3;
   W is —C(RₓR_{y})ₙ—, wherein n is an integer of 1 to 3 and Rₓ and R_{y} can be independently selected from hydrogen, alkyl, alkenyl, alkynyl, cycloalkyl, aryl, heteroaryl or heterocyclyl;
   E can be —(CRₓR_{y})ₘ— or —C(RₓR_{y})CON(Rₓ)— (wherein m is an integer of 0 to 3 and Rₓ and R_{y} are defined as above), and when E=—(CRₓR_{y})ₘ— and m=0 to 1, then R can be

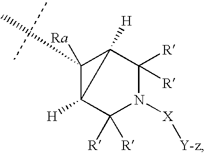

and
when E=—(CRₓR_{y})ₘ— or —C(RₓR_{y})CON(Rₓ)— and m=2-3, then R comprises one of the following Formulas:

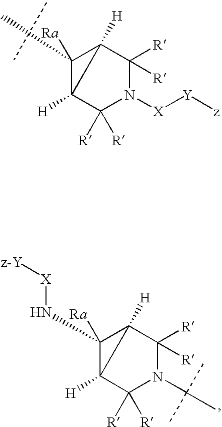

wherein
X can be no atom, —CO—, —CS—, —C(Rₓ, R_{y})ₙ— or —SO₂—, wherein 1 is an integer of 1-3; and Rₓ and R_{y} are defined as above;
   Y can be no atom, —O— or —NR_{c}—, with the proviso that Y cannot be —O— or —NRₓ— when X is C(RₓR_{y})₁ [wherein 1 is an integer of 1];
   Z is allyl, alkenyl, alkynyl, cycloalkyl, aryl, heteroaryl or heterocyclyl;
   Rₐ is hydrogen, alkyl or aryl; and
   R′ is Rₓ, —CN, carboxy, halogen, carbonyl, or amino.

In one embodiment, the invention relates to compounds of general Formula Ia,

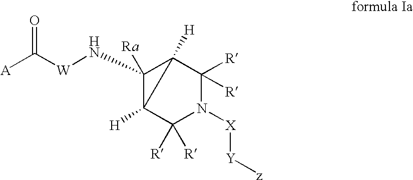

including pharmaceutically acceptable salts, pharmaceutically acceptable solvates, enantiomers, diastereomers, polymorphs, prodrugs, metabolites or N-oxides thereof,
wherein A, W, Rₐ, R′, X, Y and Z are defined as above.

In another embodiment, the invention relates to compounds of general Formula Ib,

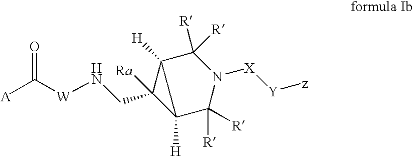

including pharmaceutically acceptable salts, pharmaceutically acceptable solvates, enantiomers, diastereomers, polymorphs, prodrugs, metabolites or N-oxides thereof,
wherein A, W, Rₐ, R′, X, Y and Z are defined as above.

In yet another embodiment, the invention relates to compounds of general Formula Ic,

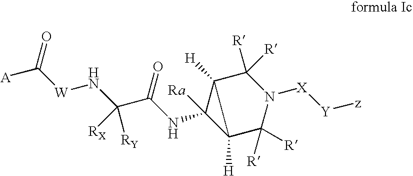

including pharmaceutically acceptable salts, pharmaceutically acceptable solvates, enantiomers, diastereomers, polymorphs, prodrugs, metabolites or N-oxides thereof,
wherein A, W, Rₐ, R′, Rₓ, R_{y}, X, Y and Z are defined as above.

In yet other embodiment, the invention encompasses compounds that include, for example,
(2S)-1-(N-{3-[(4-Methylphenyl)sulphonyl]-3-azabicyclo[3.1.0]hex-6-yl}glycyl)pyrrolidine-2-carbonitrile (Compound No. 01),
(2S)-1-{N-[3-(4-Fluorobenzoyl)-3-azabicyclo[3.1.0]hex-6-yl]glycyl}pyrrolidine-2carbonitrile (Compound No. 02),
(2S)-1-[N-({3-[(4-Fluorophenyl)sulphonyl]-3-azabicyclo[3.1.0]hex-6-yl}methyl)glycyl]pyrrolidine-2-carbonitrile (Compound No. 03).
(2S)-1-[N-(3-Benzyl-3-azabicyclo[3.1.0]hex-6-yl)glycyl]pyrrolidine-2-carbonitrile (Compound No. 04),
Phenyl(2S)-6-{[2-(2-cyano-4,4-difluoropyrrolidin-1-yl)-2-oxoethyl]amino}-3-azabicyclo[3.1.0]hexane-3-carboxylate (Compound No. 05),
(2S)-4,4-Difluoro-1-(N-{3-[(4-fluorophenyl)sulphonyl]-3-azabicyclo[3.1.0]hex-6-yl}glycyl)pyrrolidine-2-carbonitrile (Compound No. 06),
4-Chlorophenyl (2S)-6-{[2-(2-cyano-4,4-difluoropyrrolidin-1-yl)-2-oxoethyl]amino}-3-azabicyclo[3.1.0]hexane-3-carboxylate (Compound No. 07),
Phenyl (2S,4S)-6-({2-[2-cyano-4-fluoropyrrolidin-1-yl]-2-oxoethyl}amino)-3-azabicyclo[3.1.0]hexane-3-carboxylate (Compound No. 08),
(2S)-1-(N-{3-[(4-Fluorophenyl)sulphonyl]-3-azabicyclo[3.1.0]hex-6-yl}glycyl)pyrrolidine-2-carbonitrile (Compound No. 09),
(2S)-1-[N-(3-Benzyl-3-azabicyclo[3.1.0]hex-6-yl)glycyl]-4,4-difluoropyrrolidine-2-carbonitrile (Compound No. 10),
(2S,4S)-4-Fluoro-1-(N-{3-[(4-fluorophenyl)sulphonyl]-3-azabicyclo[3.1.0]hex-6-yl}glycyl)pyrrolidine-2-carbonitrile (Compound No. 11),
(2S)-4-Chlorophenyl 6-{[2-(2-cyanopyrrolidin-1-yl)-2-oxoethyl]amino}-3-azabicyclo[3.1.0]hexane-3-carboxylate (Compound No. 12)
(2S,4S)-1-(N-{3-[(4-tert-Butylphenyl)sulphonyl]-3-azabicyclo[3.1.0]hex-6-yl}glycyl)-4-fluoropyrrolidine-2-carbonitrile (Compound No. 13)
(2S,4S)-4-Fluoro-1-{N-[3-(4-fluorobenzoyl)-3-azabicyclo[3.1.0]hex-6yl]glycyl}pyrrolidine-2-carbonitrile (Compound No. 14),
(2S)-4,4-Difluoro-1-(N-{3-[5-(trifluoromethyl)pyridin-2-yl]-3-azabicyclo[3.1.0]hex-6-yl}glycyl)pyrrolidine-2-carbonitrile (Compound No. 15),
(2S)-6-{[2-(2-Cyano-4,4-difluoropyrrolidin-1-yl)-2-oxoethyl]amino}-N-(4-fluorophenyl)-3-azabicyclo[3.1.0]hexane-3-carboxamide (Compound No. 16),
(2S)-4,4-Difluoro-1-{N-[3-(4-fluorobenzoyl)-3-azabicyclo[3.1.0]hex-6-yl]glycyl}pyrrolidine-2-carbonitrile (Compound No. 17),
(2S)-1-{N-[3-(2-Naphthoyl)-3-azabicyclo[3.1.0]hex-6-yl]glycyl}pyrrolidine-2-carbonitrile (Compound No. 18),
(2S)-1-{N-[3-(Quinolin-2-ylcarbonyl)-3-azabicyclo[3.1.0]hex-6-yl]glycyl}pyrrolidine-2-carbonitrile (Compound No. 19),
(2S)-1-{N-[3-(1-Adamantylcarbonyl)-3-azabicyclo[3.1.0]hex-6-yl]glycyl}pyrrolidine-2-carbonitrile (Compound No. 20),
Phenyl (2S,4S)-6-({2-[2-cyano-4-fluoropyrrolidin-1-yl]-2-oxoethyl}amino)-3-azabicyclo[3.1.0]hexane-3-carboxylate (Compound No. 21),
(2S,4S)-6-({2-[2-Cyano-4-fluoropyrrolidin-1-yl]-2-oxoethyl}amino)-N-(4-cyanophenyl)-3-azabicyclo[3.1.0]hexane-3-carboxamide (Compound No. 22),
(2S)-6-(6-{[2-(2-Cyano-4,4-difluoropyrrolidin-1-yl)-2-oxoethyl]amino}-3-azabicyclo[3.1.0]hex-3-yl)nicotinonitrile (Compound No. 23),
(2S,4S)-6-[6-({2-[2-Cyano-4-fluoropyrrolidin-1-yl]-2-oxoethyl}amino)-3-azabicyclo[3.1.0]hex-3-yl]nicotinonitrile (Compound No. 24)
(2S)—N-{2-[2-Cyanopyrrolidin-1-yl]-2-oxoethyl-N′-[3-(4-fluorobenzoyl)-3-azabicyclo[3.1.0]hex-6-yl]glycinamide (Compound No. 25)
(2S)—N-{2-[2-Cyanopyrrolidin-1-yl]-2-oxoethyl-N′-[3-(4-fluorobenzoyl)-3-azabicyclo[3.1.0]hex-6-yl]-L-valinamide (Compound No. 26)
(2S)—N-{2-[2-Cyanopyrrolidin-1-yl]-2-oxoethyl-N′-[3-(4-fluorobenzoyl)-3-azabicyclo[3.1.0]hex-6-yl]-L-phenylalaninamide (Compound No. 27)
(2S,4S)—N-(4-{[6-({2-[2-Cyano-4-fluoropyrrolidin-1-yl]-2-oxoethyl}amino)-3-azabicyclo[3.1.0]hex-3-yl]carbonyl}phenyl)-4-methylbenzenesulphonamide (Compound No. 28)
(2S,4S)—N-(4-{[6-({2-[2-Cyano-4-fluoropyrrolidin-1-yl]-2-oxoethyl}amino)-3-azabicyclo[3.1.0]hex-3-yl]carbonyl}phenyl)methanesulphonamide (Compound No. 29)
(2S,4S)—N-(4-{[6-({2-[2-Cyano-4-fluoropyrrolidin-1-yl]-2-oxoethyl}amino)-3-azabicyclo[3.1.0]hex-3-yl]carbonyl}phenyl)-4-fluorobenzenesulphonamide (Compound No. 30)
(2S,4S)-1-(4-{[6-({2-[2-Cyano-4-fluoropyrrolidin-1-yl]-2-oxoethyl}amino)-3-azabicyclo[3.1.0]hex-3-yl]carbonyl}phenyl)-3-(4-fluorophenyl)urea (Compound No. 31)
(2S,4S)—N-(4-{[6-({2-[2-Cyano-4-fluoropyrrolidin-1-yl]-2-oxoethyl}amino)-3-azabicyclo[3.1.0]hex-3-yl]carbonyl}phenyl)-4-fluorobenzamide (Compound No. 32)
(2S,4S)-4-[6-({2-[2-Cyano-4-fluoropyrrolidin-1-yl]-2-oxoethyl}amino)-3-azabicyclo[3.1.0]hex-3-yl]benzamide (Compound No. 33)
(2S,4S)-4-[6-({2-[2-Cyano-4-fluoropyrrolidin-1-yl]-2-oxoethyl}amino)-3-azabicyclo[3.1.0]hex-3-yl]-N-cyclopropylbenzamide (Compound No. 34)
(2S,4S)-4-[6-({2-[2-Cyano-4-fluoropyrrolidin-1-yl]-2-oxoethyl}amino)-3-azabicyclo[3.1.0]hex-3-yl]-N-methylbenzamide (Compound No. 35)
(2S,4S)-1-(N-{3-[4-(Dimethylamino)benzoyl]-3-azabicyclo[3.1.0]hex-6-yl}glycyl)-4-fluoropyrrolidine-2-carbonitrile (Compound No. 36)
(2S,4S)—N-(4-{[6-({2-[2-Cyano-4-fluoropyrrolidin-1-yl]-2-oxoethyl}amino)-3-azabicyclo[3.1.0]hex-3-yl]carbonyl}phenyl)acetamide (Compound No. 37)
(2S,4S)-1-{N-[3-(4-Acetylbenzoyl)-3-azabicyclo[3.1.0]hex-6-yl]glycyl}-4-fluoropyrrolidine-2-carbonitrile (Compound No. 38)
(2S,4S)-4-Fluoro-1-(N-{3-[4-(trifluoromethyl)benzoyl]-3-azabicyclo[3.1.0]hex-6-yl}glycyl)pyrrolidine-2-carbonitrile (Compound No. 39)
(2S,4S)-1-{N-[3-(4-Acetylphenyl)-3-azabicyclo[3.1.0]hex-6-yl]glycyl}-4-fluoropyrrolidine-2-carbonitrile (Compound No. 40)
Ethyl (2S,4S)-4-[6-({2-[2-Cyano-4-fluoropyrrolidin-1-yl]-2-oxoethyl}amino)-3-azabicyclo[3.1.0]hex-3-yl]benzoate (Compound No. 41)
(2S,4S)-4-Fluoro-1-(N-{3-[4-(trifluoromethoxy)benzoyl]-3-azabicyclo[3.1.0]hex-6-yl}glycyl)pyrrolidine-2-carbonitrile (Compound No. 42)
(2S,4S)-1-{N-[3-(4-tert-Butylbenzoyl)-3-azabicyclo[3.1.0]hex-6-yl]glycyl}-4-fluoropyrrolidine-2-carbonitrile (Compound No. 43)
(2S,4S)-4-Fluoro-1-{N-[3-(4-methoxybenzoyl)-3-azabicyclo[3.1.0]hex-6-yl]glycyl}pyrrolidine-2-carbonitrile (Compound No. 44)
(2S,4S)-4-Fluoro-1-{N-[3-(4-methylbenzoyl)-3-azabicyclo[3.1.0]hex-6-yl]glycyl}pyrrolidine-2-carbonitrile (Compound No. 45)
(2S,4S)-4-Fluoro-1-{N-[3-phenyl-3-azabicyclo[3.1.0]hex-6-yl]glycyl}pyrrolidine-2-carbonitrile (Compound No. 46)
(2S,4S)-1-{N-[3-(4-Cyano-1-naphthyl)-3-azabicyclo[3.1.0]hex-6-yl]glycyl}-4-fluoropyrrolidine-2-carbonitrile (Compound No. 47)
(2S,4S)-1-(N-{3-[4-Cyano-3-(trifluoromethyl)phenyl]-3-azabicyclo[3.1.0]hex-6-yl}glycyl)-4-fluoropyrrolidine-2-carbonitrile (Compound No. 48)
(2S,4S)-1-(N-{3-[2-Cyano-3-(trifluoromethyl)phenyl]-3-azabicyclo[3.1.0]hex-6-yl}glycyl)-4-fluoropyrrolidine-2-carbonitrile (Compound No. 49)
(2S,4S)-1-{N-[3-(3-Chloro-2-cyanophenyl)-3-azabicyclo[3.1.0]hex-6-yl]glycyl}-4-fluoropyrrolidine-2-carbonitrile (Compound No. 50)
(2S,4S)-1-(N-{3-[2-Cyano-4-(trifluoromethyl)phenyl]-3-azabicyclo[3.1.0]hex-6-yl}glycyl)-4-fluoropyrrolidine-2-carbonitrile (Compound No. 51)
(2S,4S)-1-{N-[3-(4-Cyano-2-fluorophenyl)-3-azabicyclo[3.1.0]hex-6-yl]glycyl}-4-fluoropyrrolidine-2-carbonitrile (Compound No. 52)
(2S,4S)-1-{N-[3-(2-Cyano-3-fluorophenyl)-3-azabicyclo[3.1.0]hex-6-yl]glycyl}-4-fluoropyrrolidine-2-carbonitrile (Compound No. 53)
(2S,4S)-1-{N-[3-(2-Cyanophenyl)-3-azabicyclo[3.1.0]hex-6-yl]glycyl-4-fluoropyrrolidine-2-carbonitrile (Compound No. 54)
(2S,4S)-1-(N-{3-[4-Cyano-2-(trifluoromethyl)phenyl]-3-azabicyclo[3.1.0]hex-6-ylglycyl)-4-fluoropyrrolidine-2-carbonitrile (Compound No. 55)
(2S,4S)-1-{N-[3-(4-Cyano-3-fluorophenyl)-3-azabicyclo[3.1.0]hex-6-yl]glycyl}-4-fluoropyrrolidine-2-carbonitrile (Compound No. 56)
(2S,4S)-1-{N-[3-(2-Chloro-4-cyanophenyl)-3-azabicyclo[3.1.0]hex-6-yl]glycyl}-4-fluoropyrrolidine-2-carbonitrile (Compound No. 57)
(2S,4S)-1-{N-[3-(4-Cyanophenyl)-3-azabicyclo[3.1.0]hex-6-yl]glycyl}-4-fluoropyrrolidine-2-carbonitrile (Compound No. 58)
(2S,4S)-1-{N-[3-(3-Chloro-4-cyanophenyl)-3-azabicyclo[3.1.0]hex-6-yl]glycyl}-4-fluoropyrrolidine-2-carbonitrile (Compound No. 59)
(2S,4S)-1-{N-[3-(2-Cyano-6-fluorophenyl)-3-azabicyclo[3.1.0]hex-6-yl]glycyl}-4-fluoropyrrolidine-2-carbonitrile (Compound No. 60)
(2S,4S)-1-{N-[3-(2-Cyano-4-fluorophenyl)-3-azabicyclo[3.1.0]hex-6-yl]glycyl}-4-fluoropyrrolidine-2-carbonitrile (Compound No. 61)
(2S,4S)-1-{N-[3-(4-Cyanobenzoyl)-3-azabicyclo[3.1.0]hex-6-yl]glycyl}-4-fluoropyrrolidine-2-carbonitrile (Compound No. 62)
(2S,4S)-4-Fluoro-1-(N-{3-[2-fluoro-3-(trifluoromethyl)benzoyl]-3-azabicyclo[3.1.0]hex-6-yl}glycyl)pyrrolidine-2-carbonitrile (Compound No. 63)
(2S,4S)-4-Fluoro-1-(N-{3-[2-fluoro-5-(trifluoromethyl)benzoyl]-3-azabicyclo[3.1.0]hex-6-yl}glycyl)pyrrolidine-2-carbonitrile (Compound No. 64)
(2S,4S)-4-Fluoro-1-(N-{3-[4-fluoro-3-(trifluoromethyl)benzoyl]-3-azabicyclo[3.0]hex-6-yl}glycyl)pyrrolidine-2-carbonitrile (Compound No. 65)
(2S,4S)-4-Fluoro-1-(N-{3-[4-fluoro-2-(trifluoromethyl)benzoyl]-3-azabicyclo[3.1.0]hex-6-yl}glycyl)pyrrolidine-2-carbonitrile (Compound No. 66)
(2S,4S)-4-Fluoro-1-(N-{3-[3-fluoro-4-(trifluoromethyl)benzoyl]-3-azabicyclo[3.1.0]hex-6-yl}glycyl)pyrrolidine-2-carbonitrile (Compound No. 67)
(2S,4S)-1-{N-[3-(3-Chloro-2-fluorobenzoyl)-3-azabicyclo[3.1.0]hex-6-yl]glycyl}-4-fluoropyrrolidine-2-carbonitrile (Compound No. 68)
(2S,4S)-1-(N-{3-[2,4-Bis(trifluoromethyl)benzoyl]-3-azabicyclo[3.0]hex-6-yl}glycyl)-4-fluoropyrrolidine-2-carbonitrile (Compound No. 69)
(2S,4S)-1-{N-[3-(2,6-Difluorobenzoyl)-3-azabicyclo[3.0]hex-6-yl]glycyl}-4-fluoropyrrolidine-2-carbonitrile (Compound No. 70)
(2S,4S)-1-{N-[3-(2,4-Difluorobenzoyl)-3-azabicyclo[3.0]hex-6-yl]glycyl}-4-fluoropyrrolidine-2-carbonitrile (Compound No. 71)
(2S,4S)-1-{N-[3-(3,4-Difluorobenzoyl)-3-azabicyclo[3.0]hex-6-yl]glycyl}-4-fluoropyrrolidine-2-carbonitrile (Compound No. 72)
(2S,4S)-1-{N-[3-(3-Chloro-2,4-difluorobenzoyl)-3-azabicyclo[3.1.0]hex-6-yl]glycyl}-4-fluoropyrrolidine-2-carbonitrile (Compound No. 73)
(2S,4S)-1-{N-[3-(2-Chloro-4,5-difluorobenzoyl)-3-azabicyclo[3.1.0]hex-6-yl]glycyl}-4-fluoropyrrolidine-2-carbonitrile (Compound No. 74)
(2S,4S)-4-Fluoro-1-{N-[3-(2,3,4-trifluorobenzoyl)-3-azabicyclo[3.1.0]hex-6-yl]glycyl}pyrrolidine-2-carbonitrile (Compound No. 75)
(2S,4S)-4-Fluoro-1-{N-[3-(3-fluorobenzoyl)-3-azabicyclo[3.1.0]hex-6-yl]glycyl}pyrrolidine-2-carbonitrile (Compound No. 76)
(2S,4S)-4-Fluoro-1-{N-[3-(3-fluorobenzoyl)-3-azabicyclo[3.1.0]hex-6-yl]glycyl}pyrrolidine-2-carbonitrile (Compound No. 77)
(2S,4S)-4-Fluoro-1-{N-[3-(2,4,5-trifluorobenzoyl)-3-azabicyclo[3.1.0]hex-6-yl]glycyl}pyrrolidine-2-carbonitrile (Compound No. 78)
(2S,4S)-1-{N-[3-(3-Chloro-4-fluorobenzoyl)-3-azabicyclo[3.1.0]hex-6-yl]glycyl}-4-fluoropyrrolidine-2-carbonitrile (Compound No. 79)
(2S,4S)-4-Fluoro-1-{N-[3-(4-fluoro-2-methylbenzoyl)-3-azabicyclo[3.1.0]hex-6-yl]glycyl}pyrrolidine-2-carbonitrile (Compound No. 80)
(2S,4S)-4-Fluoro-1-{N-[3-(3,4,5-trifluorobenzoyl)-3-azabicyclo[3.1.0]hex-6-yl]glycyl}pyrrolidine-2-carbonitrile (Compound No. 81)
(2S,4S)-1-{N-[3-(3-Cyano-5-fluorophenyl)-3-azabicyclo[3.1.0]hex-6-yl]glycyl}-4-fluoropyrrolidine-2-carbonitrile (Compound No. 82)
(2S,4S)-1-{N-[3-(2-Cyano-3,5-difluorophenyl)-3-azabicyclo[3.1.0]hex-6-yl]glycyl}-4-fluoropyrrolidine-2-carbonitrile (Compound No. 83)
(2S,4S)-1-{N-[3-(4-Cyano-3,5-difluorophenyl)-3-azabicyclo[3.1.0]hex-6-yl]glycyl}-4-fluoropyrrolidine-2-carbonitrile (Compound No. 84)
(2S,4S)-1-{N-[3-(4-Chlorobenzoyl)-3-azabicyclo[3.1.0]hex-6-yl]glycyl}-4-fluoropyrrolidine-2-carbonitrile (Compound No. 85)
(2S,4S)-1-{N-[3-(5-Chloropyridin-2-yl)-3-azabicyclo[3.1.0]hex-6-yl]glycyl}-4-fluoropyrrolidine-2-carbonitrile (Compound No. 86)
(2S,4S)-4-Fluoro-1-{N-[3-(5-nitropyridin-2-yl)-3-azabicyclo[3.1.0]hex-6-yl]glycyl}pyrrolidine-2-carbonitrile (Compound No. 87)
(2S,4S)-4-Fluoro-1-{N-[3-pyridin-2-yl-3-azabicyclo[3.1.0]hex-6-yl]glycyl}pyrrolidine-2-carbonitrile (Compound No. 88)
(2S,4S)-1-{N-[3-(3-Chloropyridin-2-yl)-3-azabicyclo[3.1.0]hex-6-yl]glycyl}-4-fluoropyrrolidine-2-carbonitrile (Compound No. 89)
(2S,4S)—N-(4-Chlorophenyl)-6-({2-[2-cyano-4-fluoropyrrolidin-1-yl]-2-oxoethyl}amino)-3-azabicyclo[3.1.0]hexane-3-carboxamide (Compound No. 90)
(2S,4S)-4-Fluoro-1-{N-[3-{[4-(trifluoromethyl)phenyl]sulfonyl}-3-azabicyclo[3.1.0]hex-6-yl]glycyl}pyrrolidine-2-carbonitrile (Compound No. 91)
(2S,4S)-1-{N-[3-(1,3-Benzothiazol-2-yl)-3-azabicyclo[3.1.0]hex-6-yl]glycyl}-4-fluoropyrrolidine-2-carbonitrile (Compound No. 92)
(2S,4S)-1-{N-[3-(1,3-Benzoxazol-2-yl)-3-azabicyclo[3.1.0]hex-6-yl]glycyl}-4-fluoropyrrolidine-2-carbonitrile (Compound No. 93)
(2S,4S)-4-Fluoro-1-(N-{3-[4-(trifluoromethyl)pyrimidin-2-yl]-3-azabicyclo[3.1.0]hex-6-yl}glycyl)pyrrolidine-2-carbonitrile (Compound No. 94)
(2S,4S)-4-Fluoro-1-{N-[3-isoquinolin-1-yl-3-azabicyclo[3.1.0]hex-6-yl]glycyl}pyrrolidine-2-carbonitrile (Compound No. 95)
(2S,4S)-4-Fluoro-1-{N-[3-quinoxalin-2-yl-3-azabicyclo[3.0]hex-6-yl]glycyl}pyrrolidine-2-carbonitrile (Compound No. 96)
(2S,4S)-4-Fluoro-1-{N-[3-(3-nitropyridin-2-yl)-3-azabicyclo[3.1.0]hex-6-yl]glycyl}pyrrolidine-2-carbonitrile (Compound No. 97)
(2S,4S)-4-Fluoro-1-{N-[3-(1,3-thiazol-2-yl)-3-azabicyclo[3.1.0]hex-6-yl]glycyl}pyrrolidine-2-carbonitrile (Compound No. 98)
(2S,4S)-1-{N-[3-(Cyclohexylcarbonyl)-3-azabicyclo[3.1.0]hex-6-yl]glycyl}-4-fluoropyrrolidine-2-carbonitrile (Compound No. 99)
(2S,4S)-4-Fluoro-1-{N-[3-(methylsulphonyl)-3-azabicyclo[3.1.0]hex-6-yl]glycyl}pyrrolidine-2-carbonitrile (Compound No. 100)
(2S)—N-(4-Chlorophenyl)-6-({2-[2-cyanopyrrolidin-1-yl]-2-oxoethyl}amino)-3-azabicyclo[3.0]hexane-3-carboxamide (Compound No. 101)
(2S,4S)-1-(N-{3-[(4-Bromophenyl)sulphonyl]-3-azabicyclo[3.1.0]hex-6-yl}glycyl)-4-fluoropyrrolidine-2-carbonitrile (Compound No. 102)
(2S,4S)-2-[6-({2-[2-Cyano-4-fluoropyrrolidin-1-yl]-2-oxoethyl}amino)-3-azabicyclo[3.1.0]hex-3-yl]nicotinonitrile (Compound No. 103)
(2S,4S)-4-Fluoro-1-{N-[3-pyrimidin-2-yl-3-azabicyclo[3.1.0]hex-6-yl]glycyl}pyrrolidine-2-carbonitrile (Compound No. 104)
(2S,4S)-4-Fluoro-1-(N-{3-[5-(trifluoromethyl)pyridin-2-yl]-3-azabicyclo[3.0]hex-6-yl}glycyl)pyrrolidine-2-carbonitrile (Compound No. 105)
(2S,4S)-1-(N-{3-[(4-Cyanophenyl)sulphonyl]-3-azabicyclo[3.1.0]hex-6-yl}glycyl)-4-fluoropyrrolidine-2-carbonitrile (Compound No. 106)
(2S)-1-{N-[3-(3-Methoxybenzoyl)-3-azabicyclo[3.1.0]hex-6-yl]glycyl}pyrrolidine-2-carbonitrile (Compound No. 107)
(2S)-1-{N-[3-(Biphenyl-4-ylcarbonyl)-3-azabicyclo[3.1.0]hex-6-yl]glycyl}pyrrolidine-2-carbonitrile (Compound No. 108)
3-(4-Fluorobenzoyl)-N-[2-oxo-2-(1,3-thiazolidin-3-yl)ethyl]-3-azabicyclo[3.1.0]hexan-6-amine (Compound No. 109)
6-[6-{[2-Oxo-2-(1,3-thiazolidin-3-yl)ethyl]amino}-3-azabicyclo[3.1.0]hex-3-yl]nicotinonitrile (Compound No. 110)
N-(4-Fluorophenyl)-6-{[2-oxo-2-(1,3-thiazolidin-3-yl)ethyl]amino}-3-azabicyclo[3.1.0]hexane-3-carboxamide (Compound No. 111)
(2S)-1-{N-[3-(4-Chlorobenzoyl)-3-azabicyclo[3.1.0]hex-6-yl]glycyl}pyrrolidine-2-carbonitrile (Compound No. 112)
(2S)-1-(N-{3-[(Benzyloxy)acetyl]-3-azabicyclo[3.1.0]hex-6-yl}glycyl)pyrrolidine-2-carbonitrile (Compound No. 113)
(2S)-1-{N-[3-(3,4-Dichlorobenzoyl)-3-azabicyclo[3.1.0]hex-6-yl]glycyl}pyrrolidine-2-carbonitrile (Compound No. 114)
(2S)-1-{N-[3-(2,6-Dimethoxybenzoyl)-3-azabicyclo[3.1.0]hex-6-yl]glycyl}pyrrolidine-2-carbonitrile (Compound No. 115)
(2S)-1-{N-[3-(4-Methoxybenzoyl)-3-azabicyclo[3.1.0]hex-6-yl]glycyl}pyrrolidine-2-carbonitrile (Compound No. 116)
(2S)-1-(N-{3-[(4-Methoxyphenyl)acetyl]-3-azabicyclo[3.1.0]hex-6-yl}glycyl)pyrrolidine-2-carbonitrile (Compound No. 117)
(2S)-1-{N-[3-(4-tert-Butylbenzoyl)-3-azabicyclo[3.1.0]hex-6-yl]glycyl}pyrrolidine-2-carbonitrile (Compound No. 118)
(2S)-1-{N-[3-(2,4-Dichlorobenzoyl)-3-azabicyclo[3.1.0]hex-6-yl]glycyl}pyrrolidine-2-carbonitrile (Compound No. 119)
(2S)-1-{N-[3-(4-Cyanobenzoyl)-3-azabicyclo[3.1.0]hex-6-yl]glycyl}pyrrolidine-2-carbonitrile (Compound No. 120)
(2S)-1-{N-[3-(3-Chlorobenzoyl)-3-azabicyclo[3.1.0]hex-6-yl]glycyl}pyrrolidine-2-carbonitrile (Compound No. 121)
(2S)-1-(N-{3-[4-(Trifluoromethoxy)benzoyl]-3-azabicyclo[3.1.0]hex-6-yl}glycyl)pyrrolidine-2-carbonitrile (Compound No. 122),
(2S)-1-{N-[3-(2,6-Difluorobenzoyl)-3-azabicyclo[3.1.0]hex-6-yl]glycyl}pyrrolidine-2-carbonitrile (Compound No. 123),
(2S)-1-{N-[3-(4-Methylbenzoyl)-3-azabicyclo[3.1.0]hex-6-yl]glycyl}pyrrolidine-2-carbonitrile (Compound No. 124),
(2S,4S)—N-{4-[6-({2-[2-Cyano-4-fluoropyrrolidin-1-yl]-2-oxoethyl}amino)-3-azabicyclo[3.1.0]hex-3-yl]phenyl}methanesulphonamide (Compound No. 125)
(2S,4S)—N-{4-[6-({2-[2-Cyano-4-fluoropyrrolidin-1-yl]-2-oxoethyl}amino)-3-azabicyclo[3.1.0]hex-3-yl]phenyl}-4-methylbenzenesulphonamide (Compound No. 126), or
(2S,4S, 5S)-1-{N-[3-(4-Methylbenzoyl)-3-azabicyclo[3.1.0]hex-6-yl]glycyl}-4,5-methanopyrrolidine-2-carbonitrile (Compound No. 127).

In yet another embodiment, the present invention relates to the therapeutically effective dose of a compound of Formula I alone or in combination with one or more of other therapeutic agents used for treating metabolic disorder or related diseases. Examples of such therapeutic agents include, but are not limited to,
1) antihyperglycemic agents (a) other DPP IV inhibitors, e.g., saxagliptin, (b) insulin sensitizers, (I) PPAR agonists, for example, PPARγ agonists (e.g., rosiglitazone and pioglitazone), (II) PPARα/γ dual agonists (e.g., tesaglitazar and muraglitazar), and (III) PPAR pan-agonists (e.g., GSK 667954) (c) biguanides, e.g., metformin, (d) insulin secretagogues, for example, sulphonyl ureas (e.g., glimeperide) and non-sulphonyl ureas (e.g., repaglinide), (e) α-glucosidase inhibitors, e.g., acarbose, (f) protein tyrosine phosphatase-11βinhibitors, (g) glucokinase activators, e.g. PSN105 (h) inhibitors of 11β-hydroxysteroid dehydrogenase type 1, (i) glucagon receptor antagonists, (j) GLP-1 and GLP-1 receptor agonists, e.g. liraglutide (k) insulin or insulin mimetics, (l) GIP and GIP receptor agonists (m) PACAP and PACAP receptor agonists, (n) fructose 1,6 bisphosphatase inhibitors; (o) glucose 6 phosphates inhibitors; (p) Sodium glucose transporter 2 (SGLT2) inhibitor, e.g., Kissei—869682; (q) AMP-Activated protein kinase activators;
2) lipid modulating agents, (i) HMG-CoA reductase inhibitors, e.g., atorvastatin, simvastatin, and fluvastatin (ii) sequestrants (cholestyramine, colestipol, and dialkylaminoalkyl derivatives of a cross-linked dextran) (iii) nicotinyl alcohol, nicotinic acid or a salt thereof, (iv) inhibitors of cholesterol absorption, e.g., β-sitosterol and ezetimibe, (v) acyl CoA:cholesterol acyltransferase inhibitors, e.g., avasimibe (vi) ileal bile acid transporter inhibitors, and (vi) CETP inhibitors, e.g., torcetrapib;
3) antiobesity compounds, (i) CB1 receptor inverse agonists and antagonists, e.g., rimonabant (ii) β3 adrenergic receptor agonists, (iii) melanocortin-receptor agonists, in particular, melanocortin-4 receptor agonists, (iv) ghrelin antagonists, (v) neuropeptide Y₁ or Y₅ antagonists, (vi) melanin-concentrating hormone (MCH) receptor antagonists and (vii) fenfluramine, dexfenfluramine, phentermine, sibutramine, orlistat, and the like;
4) antihypertensive agents, (i) ACE inhibitors, e.g., enalapril, lisinopril, and quinapril, (ii) angiotensin II receptor antagonists and agonists, e.g., losartan, candesartan, irbesartan, valsartan, and eprosartan, (iii) β-blockers, and (iv) calcium channel blockers; and
5) anti-TNF agent or c-AMP raising agents, such as, for example, PDE inhibitors.
   The following definitions apply to terms as used herein:

The term “allyl,” unless otherwise specified, refers to a monoradical branched or unbranched saturated hydrocarbon chain having from 1 to 20 carbon atoms. Alkyl groups can be optionally interrupted by atom(s) or group(s) independently selected from oxygen, sulfur, a phenylene, sulphinyl, sulphonyl group or —NR_{α}—, wherein R_{β} can be hydrogen, alkyl, cycloalkyl, alkenyl, cycloalkenyl, alkynyl, aryl, acyl, aralkyl, —C(═O)OR_{λ}, SOₘR_{ψ} or —C(═O)NR_{λ}R_{π}. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, aryl, heterocyclyl, heteroaryl, (heterocyclyl)alkyl, cycloalkoxy, —CH═N—O(C₁₋₆alkyl), —CH═N—NH(C₁₋₆alkyl), —CH═N—NH(C₁₋₆alkyl)-C₁₋₆alkyl, arylthlio, thiol, alkylthio, aryloxy, nitro, aminosulfonyl, aminocarbonylamino, —NHC(═O)R_{λ}, —NR_{λ}R_{π}, —C(═O)NR_{λ}R_{π}, —NHC(═O)NR_{λ}R_{π}, —C(═O)heteroaryl, C(═O)heterocyclyl, —O—C(═O)NR_{λ}R_{π}, {wherein R_{λ} and R_{π}, are independently selected from hydrogen, halogen, hydroxy, alkyl, alkenyl, alkynyl, alkenyl, alkoxy, cycloalkyl, cycloalkenyl, aryl, aralkyl, heterocyclyl, heteroaryl, heterocyclylalkyl, heteroarylalkyl or carboxy}, nitro or —SOₘR_{ψ} (wherein m is an integer from 0-2 and R_{ψ} is hydrogen, alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, alkenyl, alkynyl, carboxy, —NR_{λ}R_{π}, —C(═O)NR_{λ}R_{π}, —OC(═O)NR_{λ}R_{π}, —NHC(═O)NR_{λ}R_{π}, hydroxy, alkoxy, halogen, CF₃, cyano, and —SOₘR_{ψ}; or an alkyl group also may be interrupted by 1-5 atoms of groups independently selected from oxygen, sulfur or —NR_{α}— (wherein R_{α}, R_{λ}, R_{π}, m and R_{ψ} are the same as defined earlier). Unless otherwise constrained by the definition, all substituents may be substituted further by 1-3 substituents selected from alkyl, alkenyl, alkynyl, carboxy, carboxyalkyl, —NR_{λ}R_{π}, —C(═O)NR_{λ}R_{π}, —O—C(═O)NR_{λ}R_{π}, hydroxy, alkoxy, halogen, CF₃, cyano, and —SOₘR_{ψ} (wherein R_{λ}, R_{π}, m and R_{ψ} are the same as defined earlier); or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group having from 2 to 20 carbon atoms with cis, trans or geminal geometry. Alkenyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkenyl is attached to a heteroatom, the double bond cannot be alpha to the heteroatom. Alkenyl groups may be substituted further with one or more substituents selected from alkyl, alkenyl, alkynyl, alkoxy, cycloalkyl, cycloalkenyl, acyl, acylamino, acyloxy, —NHC(═O)R_{λ}, —NR_{λ}R_{π}, —C(═O)NR_{λ}R_{π}, —NHC(═O)NR_{λ}R_{π}, —O—C(═O)NR_{λ}R_{π}, alkoxycarbonylamino, azido, cyano, halogen, hydroxy, oxo, keto, carboxyalkyl, thiocarbonyl, carboxy, arylthio, thiol, allylthio, aryl, aralkyl, aryloxy, heterocyclyl, heteroaryl, heterocyclyl alkyl, heteroaryl alkyl, aminosulfonyl, aminocarbonylamino, alkoxyamino, hydroxyamino, alkoxyamino, nitro or SOₘR_{ψ} (wherein R_{λ}, R_{π}, m and R_{ψ} are as defined earlier). Unless otherwise constrained by the definition, alkenyl substituents optionally may be substituted further by 1-3 substituents selected from alkyl, alkenyl, alkynyl, carboxy, hydroxy, alkoxy, halogen, —CF₃, cyano, —NR_{λ}R_{π}, —C(═O)NR_{λ}R_{π}, —O—C(═O)NR_{λ}R_{π}, and —SOₘR_{ψ} (wherein R_{λ}, R_{π}, m and RF are as defined earlier). Groups, such as ethenyl or vinyl (CH═CH₂), 1-propylene or allyl (—CH₂CH═CH₂), iso-propylene (—C(CH₃)═CH₂), bicyclo[2.2.1]heptene, and the like, exemplify this term.

The term “alkynyl,” unless otherwise specified, refers to a monoradical of an unsaturated hydrocarbon, having from 2 to 20 carbon atoms. Alkynyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkynyl groups are attached to a heteroatom, the triple bond cannot be alpha to the heteroatom. Alkynyl groups may be substituted further with one or more substituents selected from alkyl, alkenyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, arylthio, thiol, alkylthio, aryl, aralkyl, aryloxy, aminosulfonyl, aminocarbonylamino, hydroxyamino, alkoxyamino, nitro, heterocyclyl, heteroaryl, heterocyclylalkyl, heteroarylalkyl, —NHC(═O)R_{λ}, —NR_{λ}R_{π}, —NHC(═O)NR_{λ}R_{π}, —C(═O)NR_{λ}R_{π}, —O—C(═O)NR_{λ}R_{π} or —SOₘR_{ψ} (wherein R_{λ}, R_{π}, m and R_{ψ} are the same as defined earlier). Unless otherwise constrained by the definition, alkynyl substituents optionally may be substituted further by 1-3 substituents selected from alkyl, alkenyl, alkynyl, carboxy, carboxyalkyl, hydroxy, alkoxy, halogen, CF₃, —NR_{λ}R_{π}, —C(═O)NR_{λ}R_{π}, —NHC(═O)NR_{λ}R_{π}, —C(═O)NR_{λ}R_{π}, cyano or —SOₘR_{π} (wherein R_{λ}, R_{π}, m and R_{ψ} are the same as defined earlier).

The term “cycloalkyl,” unless otherwise specified, refers to cyclic allyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like or multiple ring structures, including adamantanyl, and bicyclo [2.2.1]heptane or cyclic alkyl groups to which is fused an aryl group, for example, indane, and the like. Spiro and fused ring structures can also be included. Cycloalkyl groups may be substituted further with one or more substituents selected from allyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{λ}R_{π}, —NHC(═O)NR_{λ}R_{π}, —NHC(═O)R_{λ}, —C(═O)NR_{λ}R_{π}, —O—C(═O)NR_{λ}R_{π}, nitro, heterocyclyl, heteroaryl, heterocyclylalkyl, heteroarylalkyl or SOₘR_{ψ} (wherein R_{λ}, R_{π}, m and R_{ψ} are the same as defined earlier). Unless otherwise constrained by the definition, cycloalkyl substituents optionally may be substituted further by 1-3 substituents selected from alkyl, alkenyl, alkynyl, carboxy, hydroxy, alkoxy, halogen, CF₃, —NR_{λ}R_{π}, —C(═O)NR_{λ}R_{π}, —NHC(═O)NR_{λ}R_{π}, —OC(═O)NR_{λ}R_{π}, cyano or —SOₘR_{ψ} (wherein R_{λ}, R_{π}, m and R_{ψ} are the same as defined earlier). “Cycloalkylalkyl” refers to alkyl-cycloalkyl group linked through alkyl portion, wherein the alkyl and cycloalkyl are the same as defined earlier.

The term “aryl,” unless otherwise specified, refers to aromatic system having 6 to 14 carbon atoms, wherein the ring system can be mono-, bi- or tricyclic and are carbocyclic aromatic groups. For example, aryl groups include, but are not limited to, phenyl, biphenyl, anthryl or napthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COOR_{ψ}, NHC(═O)R_{λ}, —NR_{λ}R_{π}, —C(═O)NR_{λ}R_{π}, —NHC(═O)NR_{λ}R_{π}, —O—C(═O)NR_{λ}R_{π}, —SOₘR_{ψ}, carboxy, heterocyclyl, heteroaryl, heterocyclylalkyl, heteroarylallcyl or amino carbonyl amino, mercapto, haloalkyl, optionally substituted aryl, optionally substituted heterocyclylalkyl, thioalkyl, —CONHR_{π}, —OCOR_{π}, —COR_{π}, —NHSO₂R_{π}, or —SO₂NHR_{π} (wherein R_{λ}, R_{π}, m and R_{ψ} are the same as defined earlier). Aryl groups optionally may be fused with a cycloalkyl group, wherein the cycloalkyl group may optionally contain heteroatoms selected from O, N or S. Groups such as phenyl, naphthyl, anthryl, biphenyl, and the like exemplify this term.

The term “heteroaryl,” unless otherwise specified, refers to an aromatic ring structure containing 5 or 6 ring atoms or a bicyclic or tricyclic aromatic group having from 8 to 10 ring atoms, with one or more heteroatom(s) independently selected from N, O or S optionally substituted with 1 to 4 substituent(s) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{λ}R_{π}, CH═NOH, —(CH₂)_{w}C(═O)R_{η}{wherein w is an integer from 0-4 and R_{η} is hydrogen, hydroxy, OR_{λ}, NR_{λ}R_{π}, —NHOR_{ω} or —NHOH}, —C(═O)NR_{λ}R_{π}, —NHC(═O)NR_{λ}R_{π}, —SOₘR_{ψ}, —O—C(═O)NR_{λ}R_{π}, —O—C(═O)R_{λ}, or —O—C(═O)OR_{λ} (wherein m, R_{ψ}, R_{λ} and R_{π} are as defined earlier and R_{ω} is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benztliiazinyl, benzthiazinonyl, benzoxazinyl, benzoxazinonyl, quinazonyl, carbazolyl phenothiazinyl, phenoxazinyl, benzothiazolyl or benzoxazolyl, and the like.

The term “heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, optionally substituted aryl, alkoxy, alkaryl, cyano, nitro, oxo, carboxy, optionally substituted heterocyclyl, optionally substituted heterocyclylalkyl, optionally substituted heteroaryl, —O—C(═O)R_{λ}, —O—C(═O)OR_{λ}, —C(═O)NR_{λ}R_{π}, SOₘR_{ψ}, —O—C(═O)NR_{λ}R_{π}, —NHC(═O)NR_{λ}R_{π}, —NR_{λ}R_{π}, mercapto, haloalkyl, thioalkyl, —COOR_{ψ}, —COONHR_{λ}, —COR_{λ}, —NHSO₂R_{λ} or SO₂NHR_{λ} (wherein m, R_{ψ}, R_{λ} and R_{π} are as defined earlier) or guanidine. Heterocyclyl can optionally include rings having one or more double bonds. Such ring systems can be mono-, bi- or tricyclic. Carbonyl or sulfonyl group can replace carbon atom(s) of heterocyclyl. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, benzoxazinyl, benzthiazinyl, imidazolyl, benzimidazolyl, tetrazolyl, carbaxolyl, indolyl, phenoxazinyl, phenothiazinyl, dihydropyridinyl, dihydroisoxazolyl, dihydrobenzofuryl, azabicyclohexyl, thiazolidinyl, dihydroindolyl, pyridinyl, isoindole 1,3-dione, piperidinyl, tetrahydropyranyl, piperazinyl, 3H-imidazo[4,5-b]pyridine, isoquinolinyl, 1H-pyrrolo[2,3-b]pyridine or piperazinyl and the like.

The term “oxo” refers to (C═O).

The term “carboxy” refers to —C(═O)OR_{f}.

The term “amino” refers to group —N(Rₖ)₂, (wherein each Rₖ is independently selected from hydrogen, alkyl, cycloalkyl, aryl, heteroaryl, heterocyclyl).

The term “carbonyl” refers to —C(═O)R_{f} (wherein R_{f} is the same as defined earlier).

The term “thiocarbonyl” refers to —C(═S)R_{f} (wherein R_{f} is the same as defined earlier).
The term “halogen” refers to —F, —Cl, —Br, and —I.

The term “leaving group” refers to groups that exhibit or potentially exhibit the properties of being labile under the synthetic conditions and also, of being readily separated from synthetic products under defined conditions. Examples of leaving groups include, but are not limited to, halogen (e.g., F, Cl, Br, I), triflates, tosylate, mesylates, alkoxy, thioalkoxy, or hydroxy radicals and the like.

The term “protecting groups” refers to moieties that prevent chemical reaction at a location of a molecule intended to be left unaffected during chemical modification of such molecule. Unless otherwise specified, protecting groups may be used on groups, such as hydroxy, amino, or carboxy. Examples of protecting groups are found in T. W. Greene and P. G. M. Wuts, “Protective Groups in Organic Synthesis”, 2^{nd} Ed., John Wiley and Sons, New York, N.Y., which is incorporated herein by reference. The species of the carboxylic protecting groups, amino protecting groups or hydroxy protecting groups employed are not critical, as long as the derivatised moieties/moiety is/are stable to conditions of subsequent reactions and can be removed without disrupting the remainder of the molecule.

The term “pharmaceutically acceptable salts” refers to derivatives of compounds that can be modified by forming their corresponding acid or base salts. Examples of pharmaceutically acceptable salts include, but are not limited to, mineral or organic acids salts of basic residues (such as amines), or alkali or organic salts of acidic residues (such as carboxylic acids), and the like. The term “pharmaceutically acceptable salts” also refers to a salt prepared from pharmaceutically acceptable non-toxic inorganic or organic acid. Examples of such inorganic acids include, but are not limited to, hydrochloric, hydrobromic, hydroiodic, nitrous, nitric, carbonic, sulfuric, phosphoric acid, and the like. Appropriate organic acids include, but are not limited to aliphatic, cycloaliphatic, aromatic, heterocyclic, carboxylic and sulfonic classes of organic acids, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic, methanesulfonic, ethanesulfonic, benzenesulfonic, panthenic, toluenesulfonic, 2-hydroxyethanesulfonic acid and the like.

The term “pharmaceutically acceptable solvates” refers to solvates with water (i.e., hydrates) or pharmaceutically acceptable solvents, for example, ethanol and the like. Such solvates are also encompassed within the scope of the disclosure. Furthermore, some of the crystalline forms for compounds described herein may exist as polymorphs and as such are intended to be included in the scope of the disclosure.

The present invention within its scope also includes prodrugs of the disclosed compounds of Formula I. In general, such prodrugs will be functional derivatives of these compounds, which are readily convertible in vivo into the active drugs. Conventional procedure for the selection and preparation of suitable prodrug derivatives are described, for example, in “Targeted prodrug design to optimize drug delivery”, AAPS PharmSci. (2000), 2(1), E6.

### DETAILED DESCRIPTION OF THE INVENTION

The compounds disclosed herein may be prepared by techniques well known in the art and familiar to the skilled synthetic organic chemist. In addition, the compounds of the present invention may be prepared by the following reaction sequences as depicted in, for example, Schemes I, II, III, IV and V.

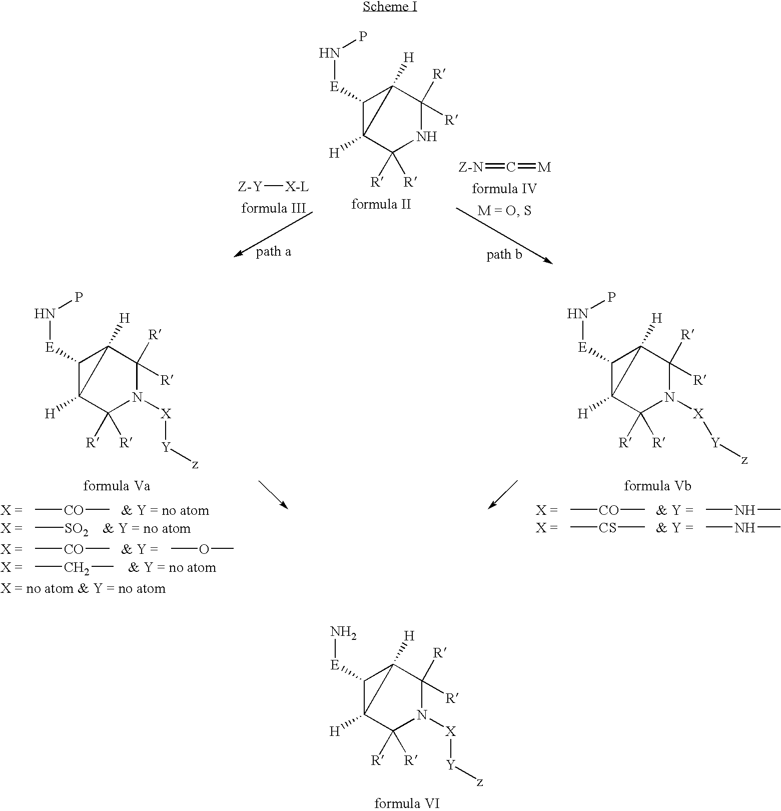

The compounds of Formula VI can be prepared, for example, following Scheme I.

Path a: A compound of Formula II (wherein P is an amino protecting group, for example, t-butyl carbamate (Boc), 9-fluorenylmethyl carbamate (Fmoc), allyloxycarbonyl or benzyl derivative; E is —(CH₂)ₘ— and m is 0-1) can be reacted with a compound of Formula III (wherein L is a leaving group such as halide or hydroxy; X is no atom, —CO—, —SO₂— or —CH₂—; Y is O or no atom; and Z is alkyl, alkenyl, alkynyl, cycloalkyl, aryl, heteroaryl or heterocyclyl) to give the compound of Formula Va.

Path b: The compound of Formula II can be reacted with a compound of Formula IV (wherein M is O or S; and Z is alkyl, alkenyl, alkynyl, cycloalkyl, aryl, heteroaryl or heterocyclyl) to form a compound of Formula Vb.

The compounds of Formula Va and Formula Vb on deprotection can yield a compound of Formula VI.

The reaction of the compound of Formula II with the compound of Formula III (wherein X is —CO—, —SO₂ or —CH₂— and Y is —O— or no atom) to give the compound of Formula Va (Path a) can be carried out in a solvent, for example, dichlioromethane, toluene, dichloroethane, tetrahydrofuran, ether or dioxane, in the presence of a base, for example, triethylamine, N,N-diisopropylethylamine or N-methylmorpholine at a temperature of 0 to 100° C.

The reaction of the compound of Formula II with the compound of Formula III (wherein X and Y are no atom) to give the compound of Formula Va (Path a) can be carried out in a solvent, for example, dimethylformamide, dioxane, tetrahydrofuran or dimethylsulphoxide, in the presence of a base, for example, potassium carbonate, triethylamine or N,N-diisopropylethylamine at a temperature of 0 to 150° C.

The reaction of the compound of Formula II with the compound of Formula IV to give a compound of Formula Vb (Path b) can be carried out in a solvent, for example, dichloromethane, toluene, dichloroethane, tetrahydrofuran, ether or dioxane, optionally, in the presence of a base, for example, potassium carbonate, triethylamine, diisopropylethylamine or N-methylmorpholine.

The deprotection of the compound of Formula Va and Formula Vb to form the compound of Formula VI can be carried out in the presence of p-toluenesulphonic acid, trifluoroacetic acid or piperidine in a solvent, for example, acetonitrile, tetrahydrofuran or dioxane, dimethylformamide or a mixture thereof. The deprotection can also be carried out by other deprotection methods known to a skilled organic chemist.

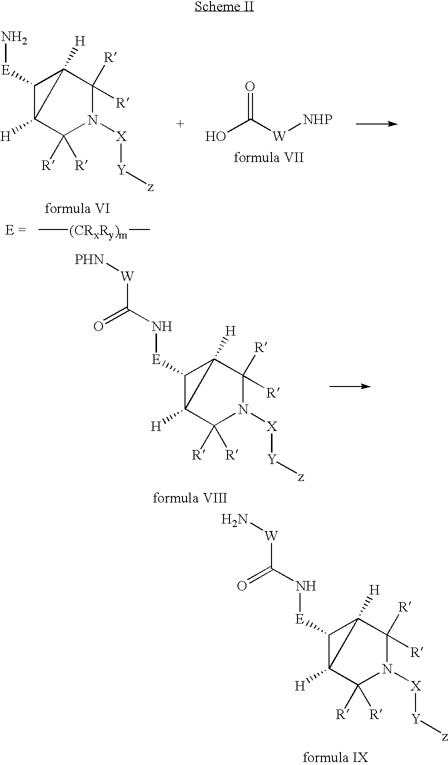

The compound of Formula IX can be prepared, for example, following Scheme II.

The compound of Formula VI can be reacted with a compound of Formula VII (wherein P is an amino protecting group and W is —C(RₓR_{y})ₙ—, wherein n is an integer of 1 to 3 and Rₓ and R_{y} can be independently selected from hydrogen, alkyl, alkenyl, alkynyl, cycloalkyl, aryl, heteroaryl or heterocyclyl) to form a compound of Formula VIII, which can be deprotected to give a compound of Formula IX.

The reaction of the compound of Formula VI with a compound of Formula VII to give a compound of Formula VIII can be carried out in a solvent, for example, tetrahydrofuran, dimethylformamide or dioxane using a coupling agent, for example, 1,3-dicyclohexylcarbodiimide (DCC), 1-ethyl-3-(3′-dimethylaminopropyl)carbodiimide hydrochloride (EDCI), N-[(dimethylamino)-1H-1,2,3-triazolo[4,5-b]pyridylmethylene]-N-methylmethanaminium hexafluorophosphate N-oxide (HATU) or benzotriazol-1-yl-N-oxy-tris(pyrrolidino)phosphonium hexafluorophosphate (PyBOP) and, optionally, a catalyst, for example, 1-hydroxybenzotriazole (HOBt), 3-hydroxy-3,4-dihydro-4-oxo-1,2,3-benzotriazine (HODhbt) or 7-aza-1-hydroxybenzotriazole (HOAt) and, optionally, with a base, for example, N,N-dimethylaminopyridine (DMAP), triethylamine, N,N-diisopropylethylamine or N-methylmorpholine. The reaction can also be carried out by any other method for amide bond formation.

The deprotection of the compound of Formula VIII to form the compound of Formula IX can be carried out under similar conditions as that of the deprotection of the compound of Formula Va to provide the compound of Formula VI.

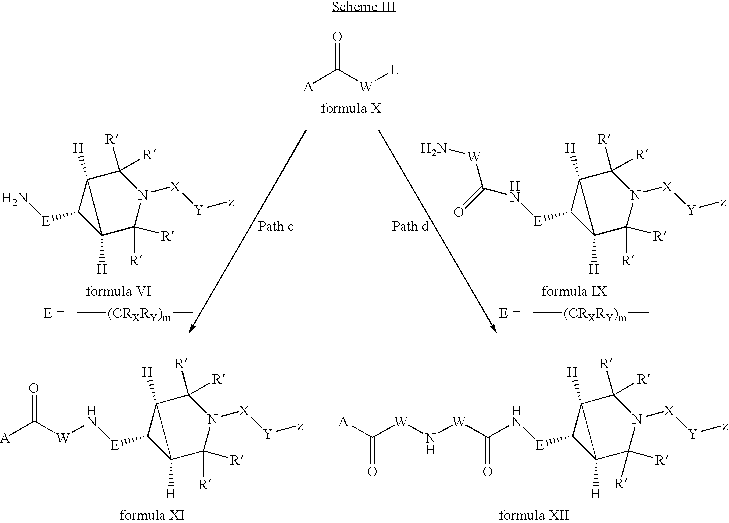

The compounds of Formula XI and XII can be prepared, for example, following Scheme III.

Path c: The compound of Formula X (wherein A can be selected from

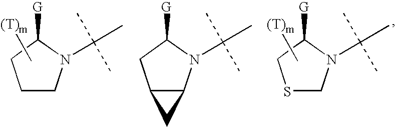

wherein
G can be selected from H, —CN, —COR₁, —CR₂═NOH, —CR₂═NR₂ or B(R₃)(R₄) (wherein R₁ is hydrogen, CF₃, alkyl, aryl or heteroaryl; R₂ is H, alkyl, aryl or heteroaryl; R₃ and R₄ can be independently selected from —OH or —OR₅ [wherein —OR₅ can be hydrolyzed to —OH and R₅ is alkyl, cycloalkyl or aryl]; If R₃ and R₄ are OR₅, then R₃ and R₄ may together form a ring of 5 to 8 atoms), and
T can be cyano, halogen, allyl, alkenyl, alkynyl, hydroxy, alkoxy, carbonyl, thiocarbonyl, and oxo and n is 0-3, W is —C(RₓR_{y})ₙ—, wherein n is an integer of 1 to 3 and Rₓ and R_{y} can be independently selected from hydrogen, alkyl, alkenyl, alkynyl, cycloalkyl, aryl, heteroaryl or heterocyclyl and L is a leaving group) is reacted with a compound of Formula VI to form a compound of Formula XI.

Path d: The compound of Formula X (wherein A, W and L are defined as earlier) is reacted with a compound of Formula IX to form a compound of Formula XII.

The above reactions (path c and path d) can be carried out in a solvent, for example, dimethyl formamide, tetrahydrofuran, dioxane, diethyl ether, dichloromethane, toluene or dichloroethene and a base, for example, potassium carbonate, triethylamine, N,N-diisopropylethylamine or N-methylmorpholine, optionally in the presence of a catalyst, like sodium iodide and tetra-n-butylammonium iodide.

The illustrative compounds, 1 to 127, were prepared following Schemes I to III.

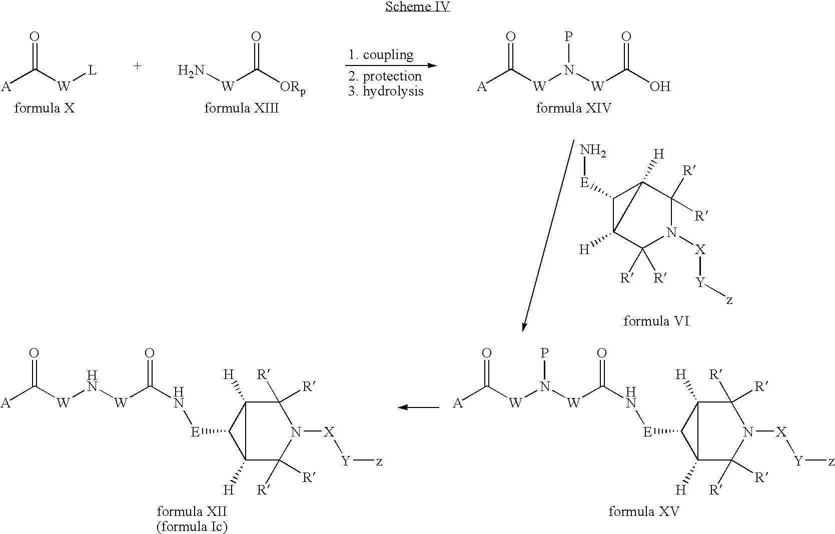

Alternatively, the compound of Formula XII can also be prepared, for example, following Scheme IV. Thus, the compound of Formula X is reacted with a compound of Formula XIII (wherein Rp is alkyl or aryl) to form a compound of Formula XIV. The compound of Formula XIV can be reacted with the compound of Formula VI to give a compound of Formula XV, which can be deprotected to give a compound of Formula XII.

The conversion of the compound of Formula X to the compound of Formula XIV can be carried out in three steps: 1) coupling of compounds of Formula X and Formula XIII in a solvent, for example, tetrahydrofuran, dimethyl formamide or dioxane, in the presence of a base, for example, potassium carbonate, triethylamine, N,N-diisopropylethylamine or N-methylmorpholine; 2) protection of amine as, for example, t-butyl carbamate (Boc), 9-fluorenylmethyl carbamate (Fmoc), allyloxycarbonyl, or benzyl derivative using conditions available to the person skilled in the art of organic synthesis; and (3) hydrolysis with a base, for example, sodium hydroxide, potassium hydroxide or lithium hydroxide in a solvent, for example, ethanol, methanol, water, tetrahydrofuran or mixtures thereof.

The reaction of a compound of Formula XIV with the compound of Formula VI to give a compound of Formula XV can be carried out under similar conditions as that of the reaction of the compound of Formula VI with the compound of Formula VII to form a compound of Formula VIII. The deprotection of the compound of Formula XV to give the compound of Formula XII (Formula Ic) can be carried out under similar conditions as that of the deprotection of the compound of Formula Va to provide the compound of Formula VI.

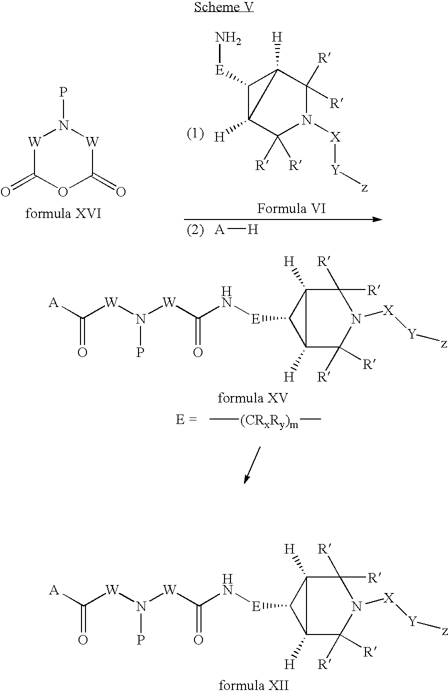

The compound of Formula XII, wherein W is —CH₂— can be prepared, for example, following Scheme V. The compound of Formula XVI (can be prepared, for example, as described in WO 2004/103993) can be reacted with the compound of Formula VI in a solvent, for example, dichloromethane, to provide an intermediate, which, in turn, can be coupled with A-H (wherein A is defined as earlier) in the presence of an amino acid coupling agent (e.g., DCC, EDCI, etc.) and optionally a catalyst (e.g., HOBt) and an organic base (e.g., N-methylmorpholine) in a solvent, for example, dimethylformamide to give a compound of Formula XV, which, in turn, can be deprotected to give the compound of Formula XII.

The deprotection of the compound of Formula XV to give the compound of Formula XII can be carried out under similar conditions as that of the deprotection of the compound of Formula Va to provide the compound of Formula VI.

In the above schemes, where specific bases, acids, solvents, coupling agents, deprotecting agents, hydrolyzing agents, metal catalysts, etc., are mentioned, it is to be understood that other acids, bases, solvents, coupling agents, deprotecting agents, hydrolyzing agents, metal catalysts etc., known to those skilled in the art may also be used. Similarly, the reaction temperature and duration of the reactions may be adjusted according to the requirements that arise during the process.

Examples set forth below demonstrate the general synthetic procedures for the preparation of representative compounds. The examples are provided to illustrate particular aspect of the disclosure and should not be constrained to limit the scope of the present invention.

### EXAMPLES

### Example 1

### Synthesis of phenyl 6-amino-3-azabicyclo[3.1.0]hexane-3-carboxylate (pTSA salt)

### Step a: Synthesis of phenyl 6-[(tert-butoxycarbonyl)amino]-3-azabicyclo[3.1.0]hexane-3-carboxylate

To a solution of tert-butyl 3-azabicyclo[3.1.0]hex-6-ylcarbamate (0.500 g, 2.50 mmol) [prepared following the procedure described in Bioorg. Med. Chem. Lett. 2004, 14(11), 2773-2776)] and triethylamine (0.73 mL, 5.30 mmol) in dichloromethane (10.0 mL) at 0° C., was added dropwise a solution of phenyl chloroformate (0.41 mL, 3.30 mmol) in dichloromethane (5.0 mL) and the reaction mixture was stirred at room temperature for about 2-3 hours and partitioned between water (10.0 mL) and dichloromethane (15.0 mL). The aqueous layer was extracted with dichloromethane (15.0 mL). The combined organic layer was washed water and brine, dried over anhydrous sodium sulphate and concentrated under reduced pressure to yield the title compound, which was used as such in the next step.

¹H NMR (400 MHz, CDCl₃): δ 1.46 (s, 9H), 1.75-1.80 (m, 2H), 2.39 (s, 1H), 3.54-3.58 (m, 1M), 3.63-3.66 (m, 1H), 3.79 (d, 1H, J=12 Hz), 3.91 (br s, 1H), 4.74 (br s, 1H, NH), δ 7.07-7.36 (m, 5H); ESI-MS (m/z): 341 (M⁺+23)

### Step b: Synthesis of phenyl 6-amino-3-azabicyclo[3.1.0]hexane-3-carboxylate (pTSA salt)

To the compound obtained from ‘step a’ in acetonitrile (7.0 mL), was added p-toluenesulphonic acid (0.713 g, 3.75 mmol). The mixture was stirred for about 12 hours at room temperature. The solvent was evaporated and the residue taken in ethyl acetate. The mixture was stirred for about 30 min., and the precipitated solid filtered, washed with cold ethyl acetate and dried to yield the title compound (0.93 g, 90%). In those cases, where the solid did not precipitate (semi-solid) in step b, the solvent was decanted. Fresh ethyl acetate was added and, after stirring for 5 min., the solvent was decanted and the resulting semi-solid was dried in vacuo to afford the pure product.

¹H NMR (400 MHz, DMSO-d₆+D₂O): δ 2.07 (m, 2H), 2.38 (s, 3H), 2.62 (s, 1H), 3.56-3.59 (m, 1H), 3.68-3.73 (m, 2H), 3.84 (d, 1H, J=8.0 Hz), 7.16 (d, 2H, J=8.0 Hz), 7.26-7.31 (m, 3H), 7.45-7.48 (m, 2H), 7.61 (d, 2H, J=8.0 Hz); ESI-MS (m/z): 219 (M⁺+1, free amine)

The following illustrative intermediates were prepared by following the preparation of phenyl 6-amino-3-azabicyclo[3.1.0]hexane-3-carboxylate (pTSA salt) except that appropriate acyl chloride, sulphonyl chloride or chloroformate was used instead of phenyl chloroformate:
4-Chlorophenyl 6-amino-3-azabicyclo[3.1.0]hexane-3-carboxylate (pTSA Salt)
3-(4-Fluorobenzoyl)-3-azabicyclo[3.1.0]hexan-6-amine (pTSA salt)
3-[(4-Methylphenyl)sulphonyl]-3-azabicyclo[3.1.0]hexan-6-amine (pTSA salt)
3-[(4-Fluorophenyl)sulphonyl]-3-azabicyclo[3.1.0]hexan-6-amine (pTSA salt)
3-[(4-tert-Butylphenyl)sulphonyl]-3-azabicyclo[3.1.0]hexan-6-amine (pTSA salt)
1-{3-[(4-Fluorophenyl)sulphonyl]-3-azabicyclo[3.1.0]hex-6-yl}methanamine (pTSA salt)
3-(Cyclohexylcarbonyl)-3-azabicyclo[3.1.0]hexan-6-amine (pTSA salt)
3-(4-Methylbenzoyl)-3-azabicyclo[3.1.0]hexan-6-amine (pTSA salt)
3-[4-(Trifluoromethoxy)benzoyl]-3-azabicyclo[3.1.0]hexan-6-amine (pTSA salt)
3-(3-Chlorobenzoyl)-3-azabicyclo[3.1.0]hexan-6-amine (pTSA salt)
3-(4-Cyanobenzoyl)-3-azabicyclo[3.1.0]hexan-6-amine (pTSA salt)
3-(2,4-Dichlorobenzoyl)-3-azabicyclo[3.1.0]hexan-6-amine (pTSA salt)
3-(4-tert-Butylbenzoyl)-3-azabicyclo[3.1.0]hexan-6-amine (pTSA salt)
3-[(4-Methoxyphenyl)acetyl]-3-azabicyclo[3.1.0]hexan-6-amine (pTSA salt)
3-(4-Methoxybenzoyl)-3-azabicyclo[3.1.0]hexan-6-amine (pTSA salt)
3-(4-Chlorobenzoyl)-3-azabicyclo[3.1.0]hexan-6-amine (pTSA salt)
3-(2,6-Dimethoxybenzoyl)-3-azabicyclo[3.1.0]hexan-6-amine (pTSA salt)
3-(3,4-Dichlorobenzoyl)-3-azabicyclo[3.1.0]hexan-6-amine (pTSA salt)
3-(Benzyloxyacetyl)-3-azabicyclo[3.1.0]hexan-6-amine (pTSA salt)
3-(4-Phenylbenzoyl)-3-azabicyclo[3.1.0]hexan-6-amine (pTSA salt)
3-(3-Methoxybenzoyl)-3-azabicyclo[3.1.0]hexan-6-amine (pTSA salt)
3-(4-Cyanophenylsulphonyl)-3-azabicyclo[3.1.0]hexan-6-amine (pTSA salt)
3-(4-Bromophenylsulphonyl)-3-azabicyclo[3.1.0]hexan-6-amine (pTSA salt)
3-(Methylsulfonyl)-3-azabicyclo[3.1.0]hexan-6-amine (pTSA salt)
3-(4-{Trifluoromethyl}phenylsulphonyl)-3-azabicyclo[3.1.0]hexan-6-amine (pTSA salt)
3-(3,4,5-Trifluorobenzoyl)-3-azabicyclo[3.1.0]hexan-6-amine (pTSA salt)
3-(4-Fluoro-2-methylbenzoyl)-3-azabicyclo[3.1.0]hexan-6-amine &TSA salt)
3-(3-Chloro-4-fluorobenzoyl)-3-azabicyclo[3.1.0]hexan-6-amine (pTSA salt)
3-(2,4,5-Trifluorobenzoyl)-3-azabicyclo[3.1.0]hexan-6-amine (pTSA salt)
3-(4-Fluoro-3-methylbenzoyl)-3-azabicyclo[3.1.0]hexan-6-amine (pTSA salt)
3-(3-Fluorobenzoyl)-3-azabicyclo[3.1.0]hexan-6-amine (pTSA salt)
3-(2,3,4-Trifluorobenzoyl)-3-azabicyclo[3.1.0]hexan-6-amine (pTSA salt)
3-(2-Chloro-4,5-difluorobenzoyl)-3-azabicyclo[3.1.0]hexan-6-amine (pTSA salt)
3-(3-Chloro-2,4-difluorobenzoyl)-3-azabicyclo[3.1.0]hexan-6-amine (pTSA salt)
3-(3,4-Difluorobenzoyl)-3-azabicyclo[3.1.0]hexan-6-amine (pTSA salt)
3-(2,4-Difluorobenzoyl)-3-azabicyclo[3.1.0]hexan-6-amine (pTSA salt)
3-(2,6-Difluorobenzoyl)-3-azabicyclo[3.1.0]hexan-6-amine (pTSA salt)
3-[2,4-bis(trifluoromethyl)benzoyl]-3-azabicyclo[3.1.0]hexan-6-amine(pTSA salt)
3-(3-Chloro-2-fluorobenzoyl)-3-azabicyclo[3.1.0]hexan-6-amine (pTSA salt)
3-[3-Fluoro-4-(trifluoromethyl)benzoyl]-3-azabicyclo[3.1.0]hexan-6-amine (pTSA salt)
3-[4-Fluoro-2-(trifluoromethyl)benzoyl]-3-azabicyclo[3.1.0]hexan-6-amine (pTSA salt)
3-(4-Fluoro-3-[trifluoromethyl]benzoyl)-3-azabicyclo[3.1.0]hexan-6-amine (pTSA salt)
3-(2-Fluoro-5-[trifluoromethyl]benzoyl)-3-azabicyclo[3.1.0]hexan-6-amine (pTSA salt)
3-(2-Fluoro-3-[trifluoromethyl]benzoyl)-3-azabicyclo[3.1.0]hexan-6-amine (pTSA salt)
3-(4-Acetylbenzoyl)-3-azabicyclo[3.1.0]hexan-6-amine (pTSA salt)
3-([4-Trifluoromethyl]benzoyl)-3-azabicyclo[3.1.0]hexan-6-amine (pTSA salt)
3-(4-Acetamidobenzoyl)-3-azabicyclo[3.1.0]hexan-6-amine (pTSA salt)
3-(4-{N,N-Dimethylamino}benzoyl)-3-azabicyclo[3.1.0]hexan-6-amine (pTSA salt)
3-(4-[{4-Fluorophenylsulphonyl}amino]benzoyl)-3-azabicyclo[3.1.0]hexan-6-amine (pTSA salt)
3-(4-[{N-Methanesulphonyl}amino]benzoyl)-3-azabicyclo[3.1.0]hexan-6-amine (pTSA salt)
3-(4-[{4-Methylphenylsulphonyl}amino]benzoyl)-3-azabicyclo[3.1.0]hexan-6-amine (pTSA salt)
3-(Adamantan-1-ylcarbonyl)-3-azabicyclo[3.1.0]hexan-6-amine (pTSA salt)
3-(Quinolin-2-ylcarbonyl)-3-azabicyclo[3.1.0]hexan-6-amine (pTSA salt)
3-(2-Naphthoyl)-3-azabicyclo[3.1.0]hexan-6-amine (pTSA salt)

### Example 2

### Synthesis of 3-[5-(trifluoromethyl)pyridin-2-yl]-3-azabicyclo[3.1.0]hexan-6-amine (pTSA salt)

### Step a: Synthesis of tert-butyl [3-(5-trifluoropyridin-2-yl)-3-azabicyclo[3.1.0]hex-6-yl] carbamate

A solution of tert-butyl 3-azabicyclo[3.1.0]hex-6-ylcarbamate (0.500 g, 2.50 mmol) and 2-chloro-5-(trifluoromethyl)pyridine (0.38 mL, 3.0 mmol) in dimethylformamide (5.0 mL) was heated at 80° C. for about 6 hours. The solvent was removed under vacuum, and the residue partitioned between dichloromethane (30.0 mL) and water (20.0 mL). The organic layer was washed with brine, dried over anhydrous sodium sulphate and concentrated under reduced pressure. The residue was purified by column chromatography using 30% ethyl acetate in hexane as eluant (silica gel 100-200 mesh) to yield the title compound.

### Step b: Synthesis of 3-[5-(trifluoromethyl)pyridin-2-yl]-3-azabicyclo[3.1.0]hexan-6-amine (pTSA salt)

To the compound obtained from ‘step a’ in acetonitrile (7.0 mL), was added pTSA (0.978 g, 5.14 mmol) at room temperature. The mixture was stirred for about 12 hours. The solvent was evaporated. The crude mixture was taken in ethyl acetate and stirred for about 30 minutes. The precipitated solid was filtered, washed with cold ethyl acetate and dried under reduced pressure to afford the title compound (0.76 gm, 69%).

¹H NMR (400 MHz, MeOH-d₄): δ 2.36 (s, 5H), 2.65 (s, 1H), 3.87 (d, 2H, J=8.0 Hz), 3.97 (d, 2H, J=8.0 Hz), 7.13 (d, 1H, J=8.0 Hz), 7.23 (d, 2H, J=8.0 Hz), 7.68 (d, 2H, J=8.0 Hz), 8.08 (d, 1H, J=8.0 Hz), 8.26 (s, 1H); ESI-MS (m/z): 244 (M⁺+1, free amine)

The following intermediates were prepared by following the preparation of 3-[5-(trifluoromethyl)pyridin-2-yl]-3-azabicyclo[3.1.0]hexan-6-amine (pTSA salt) by replacement of 2-chloro-5-trifluoromethylpyridine with appropriate haloheteroaryls or haloaryls at different temperatures (50-140° C.) and times (8-12 hours):
6-(6-Amino-3-azabicyclo[3.1.0]hex-3-yl)nicotinonitrile (pTSA salt)
3-[3-Cyanopyridin-2-yl]-3-azabicyclo[3.1.0]hexan-6-amine (pTSA salt)
3-[3-Chloropyridin-2-yl]-3-azabicyclo[3.1.0]hexan-6-amine (pTSA salt)
3-[5-Chloropyridin-2-yl]-3-azabicyclo[3.1.0]hexan-6-amine (pTSA salt)
3-[Pyridin-2-yl]-3-azabicyclo[3.1.0]hexan-6-amine (pTSA salt)
3-[3-Nitropyridin-2-yl]-3-azabicyclo[3.1.0]hexan-6-amine (pTSA salt)
3-[5-Nitropyridin-2-yl]-3-azabicyclo[3.1.0]hexan-6-amine (pTSA salt)
3-[Pyridimidin-2-yl]-3-azabicyclo[3.1.0]hexan-6-amine (pTSA salt)
3-[4-[Trifluoromethyl]pyridimidin-2-yl]-3-azabicyclo[3.1.0]hexan-6-amine (pTSA salt)
3-[Thiazol-2-yl]-3-azabicyclo[3.1.0]hexan-6-amine (pTSA salt)
3-[Quinoxalin-2-yl]-3-azabicyclo[3.1.0]hexan-6-amine (pTSA salt)
3-[Isoquinolin-1-yl]-3-azabicyclo[3.1.0]hexan-6-amine (pTSA salt)
3-[1,3-Benzoxzol-2-yl]-3-azabicyclo[3.1.0]hexan-6-amine (pTSA salt)
3-[1,3-Benzthiazol-2-yl]-3-azabicyclo[3.1.0]hexan-6-amine (pTSA salt)
3-[4-Cyano-3,5-difluorophenyl]-3-azabicyclo[3.1.0]hexan-6-amine (pTSA salt)
3-[2-Cyano-3,5-difluorophenyl]-3-azabicyclo[3.1.0]hexan-6-amine (pTSA salt)
3-[5-Cyano-3-fluorophenyl]-3-azabicyclo[3.1.0]hexan-6-amine (pTSA salt)
3-[2-Cyano-4-fluorophenyl]-3-azabicyclo[3.1.0]hexan-6-amine (pTSA salt)
3-[2-Cyano-6-fluorophenyl]-3-azabicyclo[3.1.0]hexan-6-amine (pTSA salt)
3-[5-Chloro-4-cyanophenyl]-3-azabicyclo[3.1.0]hexan-6-amine (pTSA salt)
3-[4-Cyanophenyl]-3-azabicyclo[3.1.0]hexan-6-amine (pTSA salt)
3-[2-Chloro-4-cyanophenyl]-3-azabicyclo[3.1.0]hexan-6-amine (pTSA salt)
3-[3-Chloro-4-cyanophenyl]-3-azabicyclo[3.1.0]hexan-6-amine (pTSA salt)
3-[4-Cyano-3-fluorophenyl]-3-azabicyclo[3.1.0]hexan-6-amine (pTSA salt)
3-[4-Cyano-2-{trifluoromethyl]phenyl}-3-azabicyclo[3.1.0]hexan-6-amine (pTSA salt)
3-[2-Cyanophenyl]-3-azabicyclo[3.1.0]hexan-6-amine (pTSA salt)
3-[2-Cyano-3-fluorophenyl]-3-azabicyclo[3.1.0]hexan-6-amine (pTSA salt)
3-[4-Cyano-2-fluorophenyl]-3-azabicyclo[3.1.0]hexan-6-amine (pTSA salt)
3-[2-Cyano-4-{trifluoromethyl}phenyl]-3-azabicyclo[3.1.0]hexan-6-amine (pTSA salt)
3-[3-Chloro-2-cyanophenyl]-3-azabicyclo[3.1.0]hexan-6-amine (pTSA salt)
3-[2-Cyano-3-{trifluoromethyl}phenyl]-3-azabicyclo[3.1.0]hexan-6-amine (pTSA salt)
3-[4-Cyano-3-{trifluoromethyl}phenyl]-3-azabicyclo[3.1.0]hexan-6-amine (pTSA salt)
3-[4-cyanonaphthyl]-3-azabicyclo[3.1.0]hexan-6-amine (pTSA salt)
3-[4-acetylphenyl]-3-azabicyclo[3.1.0]hexan-6-amine (pTSA salt)
3-(4-{Ethoxycarbonyl}phenyl)-3-azabicyclo[3.1.0]hexan-6-amine (pTSA salt)

### Example 3

### Synthesis of 6-amino-3-azabicyclo[3.1.0]hexane-3-N-(4-fluorophenyl)carboxamide (pTSA salt)

### Step a: Synthesis of tert-butyl (3-{[(4-fluorophenyl)amino]carbonyl}-3-azabicyclo[3.1.0]hex-6-yl)carbamate

To a solution of tert-butyl 3-azabicyclo[3.1.0]hex-6-ylcarbamate (0.500 g, 2.50 mmol) in dichloromethane (10.0 mL) at 0° C., was added dropwise a solution of 4-fluorophenyl isocyanate (0.34 mL, 3.0 mmol) in dichloromethane (5.0 mL) and stirred at 0° C. for about 3 hours. The reaction mixture was partitioned between water (10.0 mL) and dichloromethane (20.0 mL). The organic layer was washed with brine, dried over anhydrous sodium sulphate and concentrated under reduced pressure to yield the title product, which was used directly in the next step.

### Step b: Synthesis of 6-amino-N-(4-fluorophenyl)-3-azabicyclo[3.1.0]hexane-3-carboxamide (pTSA salt)

To the compound obtained from ‘step a’ in acetonitrile (7.0 mL), was added p-toluenesulphonic acid (0.713 g, 3.75 mmol) at room temperature. The reaction mixture was stirred for 12 hours. The solvent was evaporated and the crude mixture taken in ethyl acetate and stirred for 30 minutes. The precipitate was filtered, washed with cold ethyl acetate and dried under reduced pressure to yield the title compound (1.021 g, 95%)

¹H NMR (400 MHz, MeOH-d₄): δ 2.07 (s, 2H), 2.36 (s, 3H), 2.45 (s, 1H), 3.55 (d, 2H, J=12.0 Hz), 3.79 (d, 2H, J=12.0 Hz), 6.99 (m, 2H), 7.23 (d, 2H, J=8.0 Hz), 7.33-7.36 (m, 2H), 7.70 (d, 2H, J=8.0 Hz); ESI-MS (m/z): 236 (M⁺+1, free amine)

The following illustrative intermediates were prepared as per the procedure for phenyl 6-amino-N-(4-fluorophenyl)-3-azabicyclo[3.1.0]hexane-3-carboxamide by replacement of 4-fluorophenyl isocyanate with appropriate isocyanate:
6-Amino-N-(4-cyanophenyl)-3-azabicyclo[3.1.0]hexane-3-carboxamide
6-Amino-N-(4-chlorophenyl)-3-azabicyclo[3.1.0]hexane-3-carboxamide

### Example 4

### Synthesis of 3-phenol-3-azabicyclo[3.1.0]hexan-6-amine (pTSA salt)

### Step a: Synthesis of tert-butyl (3-phenyl-3-azabicyclo[3.1.0]hex-6-yl)carbamate

To a solution of tert-butyl 3-azabicyclo[3.1.0]hex-6-ylcarbamate (0.500 g, 2.52 mmol) in toluene (20.0 mL), was added potassium tert-butoxide (330 mg, 2.95 mmol), bromobenzene (0.444 g, 2.1 mmol), 2,2′-bis(diphenylphosphino)-1,1′-binaphthyl (BINAP) (39.3 mg, 0.063 mmol) and Pd₂(dba)₃ (40.0 mg, 0.042 mmol) at ambient temperature under argon atmosphere. The reaction mixture was heated to 80° C. for about 4 hours. The reaction mixture was allowed to cool to room temperature and then partitioned between water (20.0 mL) and ether (20.0 mL). The organic layer was washed with brine, dried over anhydrous sodium sulphate and concentrated under reduced pressure to yield the residue, which was partially purified by column chromatography (silica gel 100-200 mesh, 25% ethyl acetate in hexane).

### Step b: Synthesis of 3-phenyl-3-azabicyclo[3.1.0]hexan-6-amine (pTSA salt)

To the compound obtained from ‘step a’ (140.0 mg, 0.51 mmol) in acetonitrile (7.0 mL), was added p-toluenesulphonic acid (0.145 g, 0.77 mmol) at room temperature. The reaction mixture was stirred for 12 hours. The solvent was evaporated and the crude mixture taken in ethyl acetate and stirred for 30 minutes. The precipitate was filtered, washed with cold ethyl acetate and dried under reduced pressure to yield the title compound (211 mg, 24%)

¹H NMR (400 MHz, MeOH-d₄): δ 2.19 (s, 2H), 2.36 (s, 3H), 2.70 (s, 1H), 3.40-3.50 (m, 2H), 3.75 (d, 2H, J=11.6 Hz), 6.80-6.95 (3H), 7.20-7.32 (m, 4H), 7.68-7.70 (m, 2H);

ESI-MS (m/z): 175.1 (M⁺+1, free amine)

The following illustrative intermediates were prepared as per the procedure for 3-phenyl-3-azabicyclo[3.1.0]hexan-6-amine (pTSA salt) by replacing bromobenzene with appropriate bromoaryl derivatives.
3-(4-{Aminocarbonyl}phenyl)-3-azabicyclo[3.1.0]hexan-6-amine (pTSA salt)
3-(4-{Methylaminocarbonyl}phenyl)-3-azabicyclo[3.1.0]hexan-6-amine (pTSA salt)
3-(4-{Cyclopropylaminocarbonyl}phenyl)-3-azabicyclo[3.1.0]hexan-6-amine (pTSA salt)

### Example 5

### Synthesis of N-[4-(6-amino-3-azabicyclo[3.1.0]hex-3-yl)phenyl]-4-methylbenzenesulph onamide

### Step a: Synthesis of tert-butyl [3-(4-{[(4-methylphenyl)sulphonyl]amino}phenyl)-3-azabicyclo[3.1.0]hex-6-yl]carbamate

To a solution of 4-fluoroaniline (1.0 g, 9.0 mmol), and triethylamine (1.32 g, 18.0 mmol) in DCM (25.0 mL) under N₂ atmosphere, was added p-toluenesulphonyl chloride (1.88 g, 9.9 mmol) at 0° C. The mixture was warmed to room temperature and stirred overnight. The mixture was diluted with DCM (25.0 mL), washed with water and brine, dried over anhydrous sodium sulphate and concentrated in vacuo. The crude product (1.50 g, 5.66 mmol), obtained, was mixed with tert-butyl 3-azabicyclo[3.1.0]hex-6-ylcarbamate (1.35 g, 6.79 mmol), and potassium carbonate (1.56 g, 11.32 mmol) in DMF (5.0 mL) and heated at 130° C. overnight. The mixture was partitioned between water (30.0 mL) and ethyl acetate (30.0 mL). The organic layer was washed with water and brine, dried over anhydrous sodium sulphate and concentrated in vacuo to afford the residue, which was purified by column chromatography (silica gel 100-200 mesh, 15% ethyl acetate in hexane) to provide the title compound (650 mg, 16%).

¹H NMR (400 MHz, MeOH-d₄): δ 2.12 (s, 2H), 2.36 (s, 3H), 2.40-2.50 (m, 4H), 3.25-3.40 (m, 2H), 3.60-3.70 (m, 2H), 6.4-6.9 (m, 4H), 7.15-7.70 (m, 8H); ESI-MS (m/z): 444.17 (M⁺+1)

### Step b: Synthesis of N-[4-(6-amino-3-azabicyclo[3.1.0]hex-3-yl)phenyl]-4-methylbenzene sulphonamide (PTSA)

To the compound obtained from ‘step a’ (635 mg, 1.43 mmol) in acetonitrile (7.0 mL), was added p-toluenesulphonic acid (408 mg, 2.15 mmol) at room temperature. The reaction mixture was stirred for 12 hours. The solvent was evaporated and the crude mixture taken in ethyl acetate and stirred for 30 minutes. The precipitate was filtered, washed with cold ethyl acetate and dried under reduced pressure to yield the title compound (286 mg, 39%)

¹H NMR (400 MHz, MeOH-d₄): δ 2.12 (s, 2H), 2.41 (s, 3H), 3.65-3.88 (m, 4H), 7.22-7.30 (m, 4H), 7.68-7.75 (m, 4H), 8.07 (d, 1H, J=4.0 Hz); ESI-MS (m/z): 276.1 (M⁺+1, free amine)

The following illustrative intermediate was prepared as per the procedure for N-[4-(6-amino-3-azabicyclo[3.1.0]hex-3-yl)phenyl]-4-methylbenzenesulphonamide by replacing p-toluenesulphonyl chloride with methanesulphonyl chloride.
N-[4-(6-amino-3-azabicyclo[3.1.0]hex-3-yl)phenyl]methanesulphonamide (pTSA salt)

### Example 6

### Synthesis of (2S,4S,5S)-4,5-methanoprrolidine-2-carbonitrile (pTSA salt)

### Step a: Synthesis of (2S,4S,5S)—N-(tert-butyloxycarbonyl)-4,5-methanopyrrolidine-2-carbonitrile

To a solution of (2S,4S,5S)—N-(tert-butyloxycarbonyl)-4,5-methanopyrrolidine-2-carboxamide (5.0 g, 22.09 mmol) [prepared as per the procedure described in WO 2004/052850] in anhydrous pyridine at −20° C. under inert atmosphere, was added trifluoroacetic anhydride (12.3 mL, 88.3 mmol) dropwise. The reaction mixture was stirred at −20° C. for about 60 minutes and then at room temperature for 8 hours. Water (10.0 mL) was added. The mixture was stirred for about 30 minutes, and then extracted with ethyl acetate. The organic portion was washed with cold dilute hydrochloric acid (1N), water, dried over anhydrous sodium sulphate and concentrated in vacuo. The crude compound was purified by column chromatography using dichloromethane as eluant to yield the title compound (3.6 g, 80%).

ESI-MS (m/z): 209 (M⁺+1).

### Step b: Synthesis of (2S,4S,5S)-4,5-methanopyrrolidine-2-carbonitrile (pTSA salt)

To a solution of compound (5.0 g, 24.04 mmol) obtained from step a in dry acetonitrile (5.0 ml), was added with p-toluenesulphonic acid (6.79 g, 35.6 mmol). The mixture was stirred under inert atmosphere at room temperature overnight. The solvent was evaporated and the crude taken in cold ethyl acetate (50.0 mL). The precipitated solid was filtered, washed with cold ethyl acetate and dried in vacuo to yield the title compound (6.0 g, 90%).

¹H NMR (400 MHz MeOH-d₄): δ1.09-1.16 (m, 1H), 1.16-1.22 (m, 1H), 1.37-1.51 (m, 1H), 1.97-2.08 (m, 1H), 2.37 (s, 3H), 2.45 (dd, 1H, J=13.96 Hz, J=2.02 Hz), 2.64-2.75 (m, 1H), 3.45-3.57 (m, 1H), 4.99 (dd, 1H, J=9.84 Hz, J=2.08 Hz), 7.24 (d, 2H, J=7.9 Hz), 7.70 (d, 2H, J=8.2 Hz); ESI-MS (m/z): 109 (M⁺+1, free amine).

### Example 7

### Synthesis of N-{4-[(6-amino-3-azabicyclo[3.1.0]hex-3-yl)carbonyl]phenyl}-4-fluorobenzamide (pTSA salt)

### Step a: Synthesis of ethyl 4-[(4-fluorobenzoyl)amino]benzoate

To a solution of ethyl 4-aminobenzoate (0.500 g, 3.02 mmol) [prepared following the procedure described in Bioorg. Med. Chem. Lett. (2004), 14(11), 2773-2776)] and triethylamine (0.84 mL, 6.04 mmol) in dichloromethane (10.0 mL) at 0° C., was added dropwise a solution of 4-fluorobenzoyl chloride (0.46 mL, 3.92 mmol) in dichloromethane (5.0 mL). The reaction mixture was stirred at room temperature for about 2-3 hours and partitioned between water (10.0 mL) and dichloromethane (15.0 mL). The aqueous layer was extracted with dichloromethane (15.0 mL). The combined organic layer was washed water and brine, dried over anhydrous sodium sulphate and concentrated under reduced pressure. The residue was purified by column chromatography (20% ethyl acetate in hexane, silica gel 100-200 mesh) to yield the title compound (739 mg, 85%)

¹H NMR (400 MHz, CDCl₃): δ 1.38-1.41 (m, 3), 4.32-4.40 (m, 2H), 7.15-8.07 (m, 8H);

ESI-MS (m/z): 287.98 (M⁺+1)

### Step b: Synthesis of 4-[(4-fluorobenzoyl)amino]benzoic acid

To a solution of ethyl 4-[(4-fluorobenzoyl)amino]benzoate (0.700 g, 2.44 mmol) in the mixture of tetrahydrofuran and methanol (10.0 ml, 3:2), was added aqueous solution of lithium hydroxide [2.0 mL] (0.153 g, 3.66 mmol) at room temperature. The reaction mixture was stirred at room temperature for about 2-3 hours and then the solvent evaporated. The residue was dissolved in water (10.0 ml), acidified using dilute hydrochloric acid (1N) and then partitioned between water (10.0 mL) and dichloromethane (15.0 mL). The aqueous layer was extracted with dichloromethane (15.0 mL). The combined organic layer was washed water and brine, dried over anhydrous sodium sulphate and concentrated under reduced pressure to yield the title compound (600 mg, 95%).

¹H NMR (400 MHz, MeOH-d₄): δ 7.16-8.08 (m, 8H); ESI-MS (m/z): 259.61 (M⁺+1)

### Step c: Synthesis of tert-butyl (3-{4-[(4-fluorobenzoyl)amino]benzoyl}-3-azabicyclo[3.1.0]hex-6-yl)carbamate

To a solution of 4-[(4-fluorobenzoyl)amino]benzoic acid (0.55 g, 2.12 mmol), tert-butyl 3-azabicyclo[3.1.0]hex-6-ylcarbamate (0.42 g, 2.12 mmol), 1-hydroxybenzotriazole (0.34 g, 2.54 mmol), triethylamine (0.44 ml, 3.18 mmol) in dichloromethane (10.0 mL) at 0° C., was added in portion N-(3-dimethylaminopropyl)-N′-ethylcarbodiimide hydrochloride (0.60 g, 3.18 mmol). The reaction mixture was stirred at room temperature for about 16 hours and then partitioned between water (10.0 mL) and dichloromethane (15.0 mL). The aqueous layer was extracted with dichloromethane (15.0 mL). The combined organic layer was washed water and brine, dried over anhydrous sodium sulphate and concentrated under reduced pressure. The residue was purified by column chromatography (30% ethyl acetate in hexane, silica gel 100-200 mesh) to yield the title compound (792 mg, 85%).

¹H NMR (400 MHz, CDCl₃): δ 1.43 (s, 9H), 1.75 (m, 2H), 2.29 (s, 1H), 3.54-3.57 (m, 1H), 3.66-3.81 (m, 2H), 4.19-4.21 (m, 1H), 4.72 (br s, 1H, NH), 7.15-7.94 (m, 8H); ESI-MS (m/z): 440.14 (M⁺+1)

### Step d: Synthesis of N-{4-[(6-amino-3-azabicyclo[3.1.0]hex-3-yl)carbonyl]phenyl}-4-fluorobenzamide (pTSA)

To the compound obtained from ‘step c’ (700 mg, 1.59 mmol) in acetonitrile (7.0 mL), was added p-toluenesulphonic acid (453 mg, 2.38 mmol) at room temperature. The reaction mixture was stirred for 12 h. The solvent was evaporated and the crude mixture taken in ethyl acetate and stirred for 30 minutes. The precipitate was filtered, washed with cold ethyl acetate and dried under reduced pressure to yield the title compound (530 mg, 65%)

¹H NMR (400 MHz, MeOH-d₄): δ 2.05 (s, 2H), 2.35 (s, 3H), 2.41 (s, 1H), 3.57-3.81 (m, 3H), 4.16-4.19 (m, 1H), 7.22-7.30 (m, 4H), 7.48-8.01 (m, 8H); ESI-MS (m/z): 340.07 (M⁺+1, free amine)

The following illustrative intermediate was prepared by as per the procedure for N-{4-[(6-amino-3-azabicyclo[3.1.0]hex-3-yl)carbonyl]phenyl}-4-fluorobenzamide (PTSA salt) except that 4-fluorophenyl isocyanate was used in the first step instead of 4-fluorobenzoyl chloride.
1-{4-[(6-amino-3-azabicyclo[3.1.0]hex-3-yl)carbonyl]phenyl}-3-(4-fluorophenyl)urea

### Example 8

### Synthesis of (2S)-1-(N-{3-[(4-methylphenyl sulphonyl]-3-azabicyclo[3.1.0]hex-6-yl}glycyl)pyrrolidine-2-carbonitrile (Compound No. 01)

To a solution of 3-[(4-methylphenyl)sulphonyl]-3-azabicyclo[3.1.0]hexan-6-amine (pTSA salt) (170 mg, 0.37 mmol) in anhydrous dimethylformamide (2.0 mL) under N₂ atmosphere at room temperature was added potassium carbonate (100 mg, 0.72 mmol) and a solution of 1-(chloroacetyl)pyrrolidine-2-carbonitrile (32 mg, 0.18 mmol) (prepared following the procedure given in J. Med. Chem., (2003), 46(13), 2774-2789) in dimethylformamide (2.0 mL). The reaction mixture was stirred overnight and partitioned between water (10.0 mL) and dichloromethane (10.0 mL). The organic layer was washed with water and brine, dried over anhydrous sodium sulphate and concentrated under reduced pressure. The residue was purified by column chromatography using 3% methanol in dichloromethane as eluant (silica gel 100-200 mesh) to yield the title compound (24 mg, 33%).

¹HNMR (CDCl₃): δ 1.26 (s, 2H), 2.10-2.40 (m, 6H), 2.44 (s, 3H), 2.98-3.0 (m, 2H), 3.31-3.60 (m, 6H), 4.59 (d, 0.2H, J=8.0 Hz, CHCN, rotomer), 4.77-4.79 (d, 0.8H, J=8.0 Hz, CHCN, rotomer), 7.33 (d, 2H, J=8.0 Hz), 7.66 (d, 2H, J=8.0 Hz); ESI-MS (m/z): 389 (M⁺+1)

### Example 9

### Synthesis of (2S)-1-{N-[3-(4-fluorobenzoyl)-3-azabicyclo[3.1.0]hex-6-yl]glycyl}pyrrolidine-2-carbonitrile (Compound No. 02)

To a solution of 3-(4-fluorobenzoyl)-3-azabicyclo[3.1.0]hexan-6-amine (pTSA salt) (0.272 g, 0.7 mmol), triethylamine (0.07 mL, 0.53 mmol) in anhydrous dichloromethane (5.0 mL) under N₂ atmosphere at room temperature, was added a solution of (2S)-1-(chloroacetyl)pyrrolidine-2-carbonitrile (100 mg, 0.56 mmol) in dichloromethane (2.0 mL) and stirred overnight at room temperature. The reaction mixture was partitioned between dichloromethane (15.0 mL) and water (20.0 mL). The organic layer was washed with water and brine, dried over anhydrous sodium sulphate and concentrated under reduced pressure. The residue was purified by column chromatography using 3% methanol in dichloromethane as eluant (silica gel 100-200 mesh) to yield the title compound (80 mg, 39%).

¹H NMR (400 MHz, CDCl₃): δ 1.25 (br s, 2H), 2.10-2.40 (m, 6H), 3.40-3.65 (m, 7H), 4.13 (d, 1H, J=12.0 Hz), 4.59 (d, 0.1H, J=8.0 Hz, CHCN, rotomer), 4.70-4.80 (m, 0.9H, CHCN, rotomer), 7.07 (t, 2H, J=8.0 Hz), 7.40-7.5 (m, 2H); ESI-MS (m/z): 357 (M⁺+1).

### Example 10

### Synthesis of (2S)-1-[N-({3-[(4-fluorophenyl)sulphonyl]-3-azabicyclo[3.1.0]hex-6-yl}methlyl) glcyl]prolidine-2-carbonitrile (Compound No. 03)

To a solution of 1-{3-[(4-fluorophenyl)sulphonyl]-3-azabicyclo[3.1.0]hex-6-yl}methanamine (250 mg, 0.56 mmol) and N,N-diisopropylethylamine (0.048 mL, 0.48 mmol) in anhydrous dichloromethane (5.0 mL) under N₂ atmosphere at room temperature, was added a solution of (2S)-1-(chloroacetyl)pyrrolidine-2-carbonitrile (75 mg, 0.43 mmol) in dichloromethane (2.0 mL) and stirred overnight at room temperature. The reaction mixture was partitioned between water (10.0 mL) and dichloromethane (15.0 mL). The organic layer was washed with water and brine, dried over anhydrous sodium sulphate and concentrated under reduced pressure. The residue, obtained, was purified by column chromatography using 2% methanol in dichloromethane as eluant (silica gel 100-200 mesh) to yield the title compound (40 mg, 23%).

¹H NMR (400 MHz, CDCl₃): δ 1.0-1.1 (m, 1H), 1.38 (s, 2H), 2.1-2.4 (m, 4H), 2.5-2.6 (m, 2H), 3.05 (d, 2H, J=9.2 Hz), 3.37 (s, 2H), 3.5-3.85 (m, 4H), 4.68-4.78 (m, 1H, CHCN), 7.21 (t, 2H, J=8.4 Hz), 7.79-7.82 (m, 2H); ESI-MS (m/z): 407 (M⁺+1).

The following illustrative compounds were prepared by following one of the procedures of Example 8, Example 9 and Example 10 by coupling appropriate 3-N-substituted-3-azabicyclo[3.1.0]hexan-6-amine with (2S)-1-(chloroacetyl)pyrrolidine-2-carbonitrile or its derivative or thiazolidine:
(2S)-1-[N-(3-Benzyl-3-azabicyclo[3.1.0]hex-6-yl)glycyl]pyrrolidine-2-carbonitrile (Compound No. 04),

ESI-MS (m/z): 325 (M⁺+1),
Phenyl (2S)-6-{[2-(2-cyano-4,4-difluoropyrrolidin-1-yl)-2-oxoethyl]amino}-3-azabicyclo[3.1.0]hexane-3-carboxylate (Compound No. 05),

ESI-MS (m/z): 391 (M⁺+1);
(2S)-4,4-Difluoro-1-(N-{3-[(4-fluorophenyl)sulphonyl]-3-azabicyclo[3.1.0]hex-6-yl}glycyl)pyrrolidine-2-carbonitrile (Compound No. 06),

ESI-MS (m/z): 429 (M⁺+1);
4-Chlorophenyl (2S)-6-{[2-(2-cyano-4,4-difluoropyrrolidin-1-yl)-2-oxoethyl]amino}-3-azabicyclo[3.1.0]hexane-3-carboxylate (Compound No. 07)

ESI-MS (m/z): 425 (M⁺+1);
Phenyl (2S,4S)-6-({2-[2-cyano-4-fluoropyrrolidin-1-yl]-2-oxoethyl}amino)-3-azabicyclo[3.1.0]hexane-3-carboxylate (Compound No. 08),

ESI-MS (m/z): 373 (M⁺+1);
(2S)-1-(N-{3-[(4-Fluorophenyl)sulphonyl]-3-azabicyclo[3.1.0]hex-6-yl}glycyl)pyrrolidine-2-carbonitrile (Compound No. 09),

ESI-MS (m/z): 393 (M⁺+1);
(2S)-1-[N-(3-Benzyl-3-azabicyclo[3.1.0]hex-6-yl)glycyl]-4,4-difluoropyrrolidine-2-carbonitrile (Compound No. 10),

ESI-MS (m/z): 362 (M⁺+2);
(2S,4S)-4-Fluoro-1-(N-{3-[(4-fluorophenyl)sulphonyl]-3-azabicyclo[3.1.0]hex-6-yl}glycyl)pyrrolidine-2-carbonitrile (Compound No. 011),

ESI-MS (m/z): 411 (M⁺+1);
(2S)-4-Chlorophenyl 6-{[2-(2-cyanopyrrolidin-1-yl)-2-oxoethyl]amino}-3-azabicyclo[3.1.0]hexane-3-carboxylate (Compound No. 12),

ESI-MS (m/z): 389 (M⁺+1);
(2S,4S)-1-(N-{3-[(4-tert-Butylphenyl)sulphonyl]-3-azabicyclo[3.1.0]hex-6-yl}glycyl)-4-fluoropyrrolidine-2-carbonitrile (Compound No. 13),

ESI-MS (m/z): 373 (M⁺+1);
(2S,4S)-4-Fluoro-1-{N-[3-(4-fluorobenzoyl)-3-azabicyclo[3.1.0]hex-6-yl]glycyl}pyrrolidine-2-carbonitrile (Compound No. 14),

ESI-MS (m/z): 373 (M⁺+1);
(2S)-4,4-Difluoro-1-(N-{3-[5-(trifluoromethyl)pyridin-2-yl]-3-azabicyclo[3.1.0]hex-6-yl}glycyl)pyrrolidine-2-carbonitrile (Compound No. 15),

ESI-MS (m/z): 416 (M⁺+1);
(2S)-6-{[2-(2-Cyano-4,4-difluoropyrrolidin-1-yl)-2-oxoethyl]amino}-N-(4-fluorophenyl)-3-azabicyclo[3.1.0]hexane-3-carboxamide (Compound No. 16),

ESI-MS (m/z): 408 (M⁺+1);
(2S)-4,4-Difluoro-1-{N-[3-(4-fluorobenzoyl)-3-azabicyclo[3.1.0]hex-6-yl]glycyl}pyrrolidine-2-carbonitrile (Compound No. 17),

ESI-MS (m/z): 393 (M⁺+1)

(2S)-1-{N-[3-(2-Naphthoyl)-3-azabicyclo[3.1.0]hex-6-yl]glycyl}pyrrolidine-2-carbonitrile (Compound No. 18),

ESI-MS (m/z): 389.0 (M⁺+1)
(2S)-1-{N-[3-(Quinolin-2-ylcarbonyl)-3-azabicyclo[3.1.0]hex-6-yl]glycyl}pyrrolidine-2-carbonitrile (Compound No. 19),

ESI-MS (m/z): 390.1 (M⁺+1)
(2S)-1-{N-[3-(1-Adamantylcarbonyl)-3-azabicyclo[3.1.0]hex-6-yl]glycyl}pyrrolidine-2-carbonitrile (Compound No. 20),

ESI-MS (m/z): 415.0 (M⁺+1)
Phenyl (2S,4S)-6-({2-[2-cyano-4-fluoropyrrolidin-1-yl]-2-oxoethyl}amino)azabicyclo[3.1.0]hexane-3-carboxylate (Compound No. 21),

ESI-MS (m/z): 373 (M⁺+1)
(2S,4S)-6-({2-[2-Cyano-4-fluoropyrrolidin-1-yl]-2-oxoethyl}amino)-N-(4-cyanophenyl)-3-azabicyclo[3.1.0]hexane-3-carboxamide (Compound No. 22),

ESI-MS (m/z): 397 (M⁺+1)
(2S)-6-(6-{[2-(2-Cyano-4,4-difluoropyrrolidin-1-yl)-2-oxoethyl]amino}-3-azabicyclo[3.1.0]hex-3-yl)nicotinonitrile (Compound No. 23),

ESI-MS (m/z): 373 (M⁺+1).
(2S,4S)-6-[6-({2-[2-Cyano-4-fluoropyrrolidin-1-yl]-2-oxoethyl}amino)-3-azabicyclo[3.1.0]hex-3-yl]-nicotinonitrile (Compound No. 24)

ESI-MS (m/z): 355.2 (M⁺+1)
(2S,4S)—N-(4-{[6-({2-[2-Cyano-4-fluoropyrrolidin-1-yl]-2-oxoethyl}amino)-3-azabicyclo[3.1.0]hex-3-yl]carbonyl}phenyl)-4-methylbenzenesulphonamide (Compound No. 28),

ESI-MS (m/z): 498.3 (M⁺+1)
(2S,4S)—N-(4-{[6-({2-[2-Cyano-4-fluoropyrrolidin-1-yl]-2-oxoethyl}amino)-3-azabicyclo[3.1.0]hex-3-yl]carbonyl}phenyl)methanesulphonamide (Compound No. 29),

ESI-MS (m/z): 422.2 (M⁺+1)
(2S,4S)—N-(4-{[6-({2-[2-Cyano-4-fluoropyrrolidin-1-yl]-2-oxoethyl}amino)-3-azabicyclo[3.1.0]hex-3-yl]carbonyl}phenyl)-4-fluorobenzenesulphonamide (Compound No. 30),

ESI-MS (m/z): 530.09 ((M⁺+1)(2S,4S)-1-(4-{[6-({2-[2-Cyano-4-fluoropyrrolidin-1-yl]-2-oxoethyl}amino)-3-azabicyclo[3.1.0]hex-3-yl]carbonyl}phenyl)-3-(4-fluorophenyl)urea (Compound No. 31),

ESI-MS (m/z): 509.13 (M⁺+1)
(2S,4S)—N-(4-{[6-({2-[2-Cyano-4-fluoropyrrolidin-1-yl]-2-oxoethyl}amino)-3-azabicyclo[3.1.0]hex-3-yl]carbonyl}phenyl)-4-fluorobenzamide (Compound No. 32),

ESI-MS (m/z): 494.15 (M⁺+1)
(2S,4S)-4-[6-({2-[2-Cyano-4-fluoropyrrolidin-1-yl]-2-oxoethyl}amino)-3-azabicyclo[3.1.0]hex-3-yl]benzamide (Compound No. 33),

¹H NMR (400 MHz, MeOH-d₄): δ 1.55-1.80 (m, 2H), 2.03 (s, 1H), 2.30-2.68 (m, 2H), 3.35-4.10 (m, 8H), 4.90-5.20 (m, 1H), 5.20-5.55 (m, 1H), 7.10-7.25 (m, 2H), 7.40-7.58 (m, 2H)
(2S,4S)-4-[6-({2-[2-Cyano-4-fluoropyrrolidin-1-yl]-2-oxoethyl}amino)-3-azabicyclo[3.1.0]hex-3-yl]-N-cyclopropylbenzamide (Compound No. 34),

¹H NMR (400 MHz, MeOH-d₄): δ 0.50-0.80 (m, 4H), 1.90-2.00 (m, 2H), 2.10-2.65 (m, 3H), 2.70-2.82 (m, 1H), 3.40-4.30 (m, 8H), 4.95 (d, 1H, J=9.2 Hz), 5.08-5.40 (m, 1H), 6.45-6.60 (m, 2H), 7.60-7.70 (m, 2H)
(2S,4S)-4-[6-({2-[2-Cyano-4-fluoropyrrolidin-1-yl]-2-oxoethyl}amino)-3-azabicyclo[3.1.0]hex-3-yl]-N-methylbenzamide (Compound No. 35),

¹H NMR (400 MHz, MeOH-d₄): δ 1.90-2.70 (m, 3H), 3.30-4.30 (m, 1H), 4.95 (d, 1H, J=9.2 Hz), 5.35-5.60 (m, 1H), 6.46-6.60 (m, 2H), 7.65-7.80 (m, 2H)
(2S,4S)-1-(N-{3-[4-(Dimethylamino)benzoyl]-3-azabicyclo[3.1.0]hex-6-yl}glycyl)-4-fluoropyrrolidine-2-carbonitrile (Compound No. 36),

ESI-MS (m/z): 399.89 (M⁺+1)
(2S,4S)—N-(4-{[6-({2-[2-Cyano-4-fluoropyrrolidin-1-yl]-2-oxoethyl}amino)-3-azabicyclo[3.1.0]hex-3-yl]carbonyl}phenyl)acetamide (Compound No. 37),

ESI-MS (m/z): 413.98 (M⁺+1)
(2S,4S)-1-{N-[3-(4-Acetylbenzoyl)-3-azabicyclo[3.1.0]hex-6-yl]glycyl}-4-fluoropyrrolidine-2-carbonitrile (Compound No. 38),

ESI-MS (m/z): 399.10 (M⁺+1)
(2S,4S)-4-Fluoro-1-(N-{3-[4-(trifluoromethyl)benzoyl]-3-azabicyclo[3.1.0]hex-6-yl}glycyl)pyrrolidine-2-carbonitrile (Compound No. 39),

ESI-MS (m/z): 424.79 (M⁺+1)
(2S,4S)-1-{N-[3-(4-Acetylphenyl)-3-azabicyclo[3.1.0]hex-6-yl]glycyl}-4-fluoropyrrolidine-2-carbonitrile (Compound No. 40),

ESI-MS (m/z): 371.33 (M⁺+1)
Ethyl (2S,4S)-4-[6-({2-[2-Cyano-4-fluoropyrrolidin-1-yl]-2-oxoethyl}amino)-3-azabicyclo[3.1.0]hex-3-yl]benzoate (Compound No. 41),

ESI-MS (m/z): 401.28 (M⁺+1)
(2S,4S)-4-Fluoro-1-(N-{3-[4-(trifluoromethoxy)benzoyl]-3-azabicyclo[3.1.0]hex-6-yl}glycyl)pyrrolidine-2-carbonitrile (Compound No. 42),

ESI-MS (m/z): 440.83 (M⁺+1)
(2S,4S)-1-{N-[3-(4-tert-Butylbenzoyl)-3-azabicyclo[3.1.0]hex-6-yl]glycyl}-4-fluoropyrrolidine-2-carbonitrile (Compound No. 43),

ESI-MS (m/z): 413.29 (M⁺+1)
(2S,4S)-4-Fluoro-1-{N-[3-(4-methoxybenzoyl)-3-azabicyclo[3.1.0]hex-6-yl]glycyl}pyrrolidine-2-carbonitrile (Compound No. 44),

ESI-MS (m/z): 386.93 (M⁺+1)
(2S,4S)-4-Fluoro-1-{N-[3-(4-methylbenzoyl)-3-azabicyclo[3.1.0]hex-6-yl]glycyl}pyrrolidine-2-carbonitrile (Compound No. 45),

ESI-MS (m/z): 370.89 (M⁺+1)
(2S,4S)-4-Fluoro-1-{N-[3-phenyl-3-azabicyclo[3.1.0]hex-6-yl]glycyl}pyrrolidine-2-carbonitrile (Compound No. 46),

ESI-MS (m/z): 328.99 (M⁺+1)
(2S,4S)-1-{N-[3-(4-Cyano-1-naphthyl)-3-azabicyclo[3.1.0]hex-6-yl]glycyl}-4-fluoropyrrolidine-2-carbonitrile (Compound No. 47),

ESI-MS (m/z): 403.98 (M⁺+1)
(2S,4S)-1-(N-{3-[4-Cyano-3-(trifluoromethyl)phenyl]-3-azabicyclo[3.1.0]hex-6-yl}glycyl)-4-fluoropyrrolidine-2-carbonitrile (Compound No. 48),

ESI-MS (m/z): 421.90 (M⁺+1)
(2S,4S)-1-(N-{3-[2-Cyano-3-(trifluoromethyl)phenyl]-3-azabicyclo[3.1.0]hex-6-yl}glycyl)-4-fluoropyrrolidine-2-carbonitrile (Compound No. 49),

ESI-MS (m/z): 421.90 (M⁺+1)
(2S,4S)-1-{N-[3-(3-Chloro-2-cyanophenyl)-3-azabicyclo[3.0]hex-6-yl]glycyl}-4-fluoropyrrolidine-2-carbonitrile (Compound No. 50),

ESI-MS (m/z): 387.94 (M⁺+1)
(2S,4S)-1-(N-{3-[2-Cyano-4-(trifluoromethyl)phenyl]-3-azabicyclo[3.1.0]hex-6-yl}glycyl)-4-fluoropyrrolidine-2-carbonitrile (Compound No. 51),

ESI-MS (m/z): 421.94 (M⁺+1)
(2S,4S)-1-{N-[3-(4-Cyano-2-fluorophenyl)-3-azabicyclo[3.1.0]hex-6-yl]glycyl}-4-fluoropyrrolidine-2-carbonitrile (Compound No. 52),

ESI-MS (m/z): 371.97 (M⁺+1)
(2S,4S)-1-{N-[3-(2-Cyano-3-fluorophenyl)-3-azabicyclo[3.1.0]hex-6-yl]glycyl}-4-fluoropyrrolidine-2-carbonitrile (Compound No. 53),

ESI-MS (m/z): 371.98 (M⁺+1)
(2S,4S)-1-{N-[3-(2-Cyanophenyl)-3-azabicyclo[3.1.0]hex-6-yl]glycyl-4-fluoropyrrolidine-2-carbonitrile (Compound No. 54),

ESI-MS (m/z): 354.09 (M⁺+1)
(2S,4S)-1-(N-{3-[4-Cyano-2-(trifluoromethyl)phenyl]-3-azabicyclo[3.1.0]hex-6-ylglycyl)-4-fluoropyrrolidine-2-carbonitrile (Compound No. 55),

ESI-MS (m/z): 422.01 (M⁺+1)
(2S,4S)-1-{N-[3-(4-Cyano-3-fluorophenyl)-3-azabicyclo[3.1.0]hex-6-yl]glycyl}-4-fluoropyrrolidine-2-carbonitrile (Compound No. 56),

ESI-MS (m/z): 372.01 (M⁺+1)
(2S,4S)-1-{N-[3-(2-Chloro-4-cyanophenyl)-3-azabicyclo[3.1.0]hex-6-yl]glycyl}-4-fluoropyrrolidine-2-carbonitrile (Compound No. 57),

ESI-MS (m/z): 387.98 (M⁺+1)
(2S,4S)-1-{N-[3-(4-Cyanophenyl)-3-azabicyclo[3.1.0]hex-6-yl]glycyl}-4-fluoropyrrolidine-2-carbonitrile (Compound No. 58),

ESI-MS (m/z): 354.02 (M⁺+1)
(2S,4S)-1-{N-[3-(3-Chloro-4-cyanophenyl)-3-azabicyclo[3.1.0]hex-6-yl]glycyl}-4-fluoropyrrolidine-2-carbonitrile (Compound No. 59),

ESI-MS (m/z): 389.93 (M⁺+1)
(2S,4S)-1-{N-[3-(2-Cyano-6-fluorophenyl)-3-azabicyclo[3.1.0]hex-6-yl]glycyl}-4-fluoropyrrolidine-2-carbonitrile (Compound No. 60),

ESI-MS (m/z):372.04 (M⁺+1)
(2S,4S)-1-{N-[3-(2-Cyano-4-fluorophenyl)-3-azabicyclo[3.1.0]hex-6-yl]glycyl}-4-fluoropyrrolidine-2-carbonitrile (Compound No. 61),

ESI-MS (m/z): 372.04 (M⁺+1)
(2S,4S)-1-{N-[3-(4-Cyanobenzoyl)-3-azabicyclo[3.1.0]hex-6-yl]glycyl}-4-fluoropyrrolidine-2-carbonitrile (Compound No. 62),

ESI-MS (m/z): 381.97 (M⁺+1)
(2S,4S)-4-Fluoro-1-(N-{3-[2-fluoro-3-(trifluoromethyl)benzoyl]-3-azabicyclo[3.1.0]hex-6-yl}glycyl)pyrrolidine-2-carbonitrile (Compound No. 63),

ESI-MS (m/z): 442.92 (M⁺+1)
(2S,4S)-4-Fluoro-1-(N-{3-[2-fluoro-5-(trifluoromethyl)benzoyl]-3-azabicyclo[3.1.0]hex-6-yl}glycyl)pyrrolidine-2-carbonitrile (Compound No. 64),

ESI-MS (m/z): 442.92 (M⁺+1)
(2S,4S)-4-Fluoro-1-(N-{3-[4-fluoro-3-(trifluoromethyl)benzoyl]-3-azabicyclo[3.1.0]hex-6-yl}glycyl)pyrrolidine-2-carbonitrile (Compound No. 65),

ESI-MS (m/z): 442.92 (M⁺+1)
(2S,4S)-4-Fluoro-1-(N-{3-[4-fluoro-2-(trifluoromethyl)benzoyl]-3-azabicyclo[3.1.0]hex-6-yl}glycyl)pyrrolidine-2-carbonitrile (Compound No. 66),

ESI-MS (m/z): 442.92 (M⁺+1)
(2S,4S)-4-Fluoro-1-(N-{3-[3-fluoro-4-(trifluoromethyl)benzoyl]-3-azabicyclo[3.1.0]hex-6-yl}glycyl)pyrrolidine-2-carbonitrile (Compound No. 67),

ESI-MS (m/z): 442.92 (M⁺+1)
(2S,4S)-1-{N-[3-(3-Chloro-2-fluorobenzoyl)-3-azabicyclo[3.1.0]hex-6-yl]glycyl}-4-fluoropyrrolidine-2-carbonitrile (Compound No. 68),

ESI-MS (m/z): 408.95 (M⁺+1)
(2S,4S)-1-(N-{3-[2,4-Bis(trifluoromethyl)benzoyl]-3-azabicyclo[3.1.0]hex-6-yl}glycyl)-4-fluoropyrrolidine-2-carbonitrile (Compound No. 69),

ESI-MS (m/z): 492.90 (M⁺+1)
(2S,4S)-1-{N-[3-(2,6-Difluorobenzoyl)-3-azabicyclo[3.1.0]hex-6-yl]glycyl}-4-fluoropyrrolidine-2-carbonitrile (Compound No. 70),

ESI-MS (m/z): 392.97 (M⁺+1)
(2S,4S)-1-{N-[3-(2,4-Difluorobenzoyl)-3-azabicyclo[3.1.0]hex-6-yl]glycyl}-4-fluoropyrrolidine-2-carbonitrile (Compound No. 71),

ESI-MS (m/z): 392.97 (M⁺+1)
(2S,4S)-1-{N-[3-(3,4-Difluorobenzoyl)-3-azabicyclo[3.1.0]hex-6-yl]glycyl}-4-fluoropyrrolidine-2-carbonitrile (Compound No. 72),

ESI-MS (m/z): 393.04 (M⁺+1)
(2S,4S)-1-{N-[3-(3-Chloro-2,4-difluorobenzoyl)-3-azabicyclo[3.1.0]hex-6-yl]glycyl}-4-fluoropyrrolidine-2-carbonitrile (Compound No. 73),

ESI-MS (m/z): 426.94 (M⁺+1)
(2S,4S)-1-{N-[3-(2-Chloro-4,5-difluorobenzoyl)-3-azabicyclo[3.1.0]hex-6-yl]glycyl}-4-fluoropyrrolidine-2-carbonitrile (Compound No. 74),

ESI-MS (m/z): 426.94 (M⁺+1)
(2S,4S)-4-Fluoro-1-{N-[3-(2,3,4-trifluorobenzoyl)-3-azabicyclo[3.1.0]hex-6-yl]glycyl}pyrrolidine-2-carbonitrile (Compound No. 75),

ESI-MS (m/z): 410.96 (M⁺+1)
(2S,4S)-4-Fluoro-1-{N-[3-(3-fluorobenzoyl)-3-azabicyclo[3.0]hex-6-yl]glycyl}pyrrolidine-2-carbonitrile (Compound No. 76),

ESI-MS (m/z): 375.06 (M⁺+1)
(2S,4S)-4-Fluoro-1-{N-[3-(3-fluorobenzoyl)-3-azabicyclo[3.1.0]hex-6-yl]glycyl}pyrrolidine-2-carbonitrile (Compound No. 77),

ESI-MS (m/z): 389.01 (M⁺+1)
(2S,4S)-4-Fluoro-1-{N-[3-(2,4,5-trifluorobenzoyl)-3-azabicyclo[3.1.0]hex-6-yl]glycyl}pyrrolidine-2-carbonitrile (Compound No. 78),

ESI-MS (m/z): 410.97 (M⁺+1)
(2S,4S)-1-{N-[3-(3-Chloro-4-fluorobenzoyl)-3-azabicyclo[3.1.0]hex-6-yl]glycyl}-4-fluoropyrrolidine-2-carbonitrile (Compound No. 79),

ESI-MS (m/z): 408.95 (M⁺+1)
(2S,4S)-4-Fluoro-1-{N-[3-(4-fluoro-2-methylbenzoyl)-3-azabicyclo[3.1.0]hex-6-yl]glycyl}pyrrolidine-2-carbonitrile (Compound No. 80),

ESI-MS (m/z): 389.01 (M⁺+1)
(2S,4S)-4-Fluoro-1-{N-[3-(3,4,5-trifluorobenzoyl)-3-azabicyclo[3.1.0]hex-6-yl]glycyl}pyrrolidine-2-carbonitrile (Compound No. 81),

ESI-MS (m/z): 410.96 (M⁺+1)
(2S,4S)-1-{N-[3-(3-Cyano-5-fluorophenyl)-3-azabicyclo[3.1.0]hex-6-yl]glycyl}-4-fluoropyrrolidine-2-carbonitrile (Compound No. 82),
(2S,4S)-1-{N-[3-(2-Cyano-3,5-difluorophenyl)-3-azabicyclo[3.1.0]hex-6-yl]glycyl}-4-fluoropyrrolidine-2-carbonitrile (Compound No. 83),

ESI-MS (m/z): 390.0 (M⁺+1)
(2S,4S)-1-{N-[3-(4-Cyano-3,5-difluorophenyl)-3-azabicyclo[3.1.0]hex-6-yl]glycyl}-4-fluoropyrrolidine-2-carbonitrile (Compound No. 84),

ESI-MS (m/z): 390.0 (M⁺+1)
(2S,4S)-1-{N-[3-(4-Chlorobenzoyl)-3-azabicyclo[3.1.0]hex-6-yl]glycyl}-4-fluoropyrrolidine-2-carbonitrile (Compound No. 85),

ESI-MS (m/z): 391.22 (M⁺+1)
(2S,4S)-1-{N-[3-(5-Chloropyridin-2-yl)-3-azabicyclo[3.1.0]hex-6-yl]glycyl}-4-fluoropyrrolidine-2-carbonitrile (Compound No. 86),

ESI-MS (m/z): 364.1 (M⁺+1)
(2S,4S)-4-Fluoro-1-{N-[3-(5-nitropyridin-2-yl)-3-azabicyclo[3.1.0]hex-6-yl]glycyl}pyrrolidine-2-carbonitrile (Compound No. 87),

ESI-MS (m/z): 375.1 (M⁺+1)
(2S,4S)-4-Fluoro-1-{N-[3-pyridin-2-yl-3-azabicyclo[3.1.0]hex-6-yl]glycyl}pyrrolidine-2-carbonitrile (Compound No. 88),

ESI-MS (m/z): 330.06 (M⁺+1)
(2S,4S)-1-{N-[3-(3-Chloropyridin-2-yl)-3-azabicyclo[3.1.0]hex-6-yl]glycyl}-4-fluoropyrrolidine-2-carbonitrile (Compound No. 89),

ESI-MS (m/z): 364.1 (M⁺+1)
(2S,4S)—N-(4-Chlorophenyl)-6-({2-[2-cyano-4-fluoropyrrolidin-1-yl]-2-oxoethyl}amino)-3-azabicyclo[3.1.0]hexane-3-carboxamide (Compound No. 90),

ESI-MS (m/z): 405.93 (M⁺+1)
(2S,4S)-4-Fluoro-1-{N-[3-{[4-(trifluoromethyl)phenyl]sulfonyl}-3-azabicyclo[3.1.0]hex-6-yl]glycyl}pyrrolidine-2-carbonitrile (Compound No. 91),

ESI-MS (m/z): 461.2 (M⁺+1)
(2S,4S)-1-{N-[3-(1,3-Benzothiazol-2-yl)-3-azabicyclo[3.1.0]hex-6-yl]glycyl}-4-fluoropyrrolidine-2-carbonitrile (Compound No. 92),

ESI-MS (m/z): 386.1 (M⁺+1)
(2S,4S)-1-{N-[3-(1,3-Benzoxazol-2-yl)-3-azabicyclo[3.1.0]hex-6-yl]glycyl}-4-fluoropyrrolidine-2-carbonitrile (Compound No. 93),

ESI-MS (m/z): 370.2 (M⁺+1)
(2S,4S)-4-Fluoro-1-(N-{3-[4-(trifluoromethyl)pyrimidin-2-yl]-3-azabicyclo[3.1.0]hex-6-yl}glycyl)pyrrolidine-2-carbonitrile (Compound No. 94),

ESI-MS (m/z): 399.2 (M⁺+1)
(2S,4S)-4-Fluoro-1-{N-[3-isoquinolin-1-yl-3-azabicyclo[3.1.0]hex-6-yl]glycyl}pyrrolidine-2-carbonitrile (Compound No. 95),

ESI-MS (m/z): 380.2 (M⁺+1)
(2S,4S)-4-Fluoro-1-{N-[3-quinoxalin-2-yl-3-azabicyclo[3.1.0]hex-6-yl]glycyl}pyrrolidine-2-carbonitrile (Compound No. 96)

ESI-MS (m/z): 381.2 (M⁺+1)
(2S,4S)-4-Fluoro-1-{N-[3-(3-nitropyridin-2-yl)-3-azabicyclo[3.1.0]hex-6-yl]glycyl}pyrrolidine-2-carbonitrile (Compound No. 97)

ESI-MS (m/z): 375.3 (M⁺+1)
(2S,4S)-4-Fluoro-1-{N-[3-(1,3-thiazol-2-yl)-3-azabicyclo[3.1.0]hex-6-yl]glycyl}pyrrolidine-2-carbonitrile (Compound No. 98)

ESI-MS (m/z): 336.2 (M⁺+1)
(2S,4S)-1-{N-[3-(Cyclohexylcarbonyl)-3-azabicyclo[3.1.0]hex-6-yl]glycyl}-4-fluoropyrrolidine-2-carbonitrile (Compound No. 99)

ESI-MS (m/z): 363.2 (M⁺+1)
(2S,4S)-4-Fluoro-1-{N-[3-(methylsulphonyl)-3-azabicyclo[3.1.0]hex-6-yl]glycyl}pyrrolidine-2-carbonitrile (Compound No. 100)

ESI-MS (m/z): 331.2 (M⁺+1)
(2S)—N-(4-Chlorophenyl)-6-({2-[2-cyanopyrrolidin-1-yl]-2-oxoethyl}amino)-3-azabicyclo[3.1.0]hexane-3-carboxamide (Compound No. 101)

ESI-MS (m/z): 388.1 (M⁺+1)
(2S,4S)-1-(N-{3-[(4-Bromophenyl)sulphonyl]-3-azabicyclo[3.1.0]hex-6-yl}glycyl)-4-fluoropyrrolidine-2-carbonitrile (Compound No. 102)

ESI-MS (m/z): 471.1 (M⁺+1)
(2S,4S)-2-[6-({2-[2-Cyano-4-fluoropyrrolidin-1-yl]-2-oxoethyl}amino)-3-azabicyclo[3.1.0]hex-3-yl]nicotinonitrile (Compound No. 103)

ESI-MS (m/z): 337.1 (M⁺+1)
(2S,4S)-4-Fluoro-1-{N-[3-pyrimidin-2-yl-3-azabicyclo[3.1.0]hex-6-yl]glycyl}pyrrolidine-2-carbonitrile (Compound No. 104)

ESI-MS (m/z): 331.2 (M⁺+1)
(2S,4S)-4-Fluoro-1-(N-{3-[5-(trifluoromethyl)pyridin-2-yl]-3-azabicyclo[3.1.0]hex-6-yl}glycyl)pyrrolidine-2-carbonitrile (Compound No. 105)

ESI-MS (m/z): 398.0 (M⁺+1)
(2S,4S)-1-(N-{3-[(4-Cyanophenyl)sulphonyl]-3-azabicyclo[3.1.0]hex-6-yl}glycyl)-4-fluoropyrrolidine-2-carbonitrile (Compound No. 106)

ESI-MS (m/z): 418.1 (M⁺+1)
(2S)-1-{N-[3-(3-Methoxybenzoyl)-3-azabicyclo[3.1.0]hex-6-yl]glycyl}pyrrolidine-2-carbonitrile (Compound No. 107)

ESI-MS (m/z): 369.1 (M⁺+1)
(2S)-1-{N-[3-(Biphenyl-4-ylcarbonyl)-3-azabicyclo[3.1.0]hex-6-yl]glycyl}pyrrolidine-2-carbonitrile (Compound No. 108)

ESI-MS (m/z): 415.1 (M⁺+1)
3-(4-Fluorobenzoyl)-N-[2-oxo-2-(1,3-thiazolidin-3-yl)ethyl]-3-azabicyclo[3.1.0]hexan-6-amine (Compound No. 109)

ESI-MS (m/z): 350.2 (M⁺+1)
6-[6-{[2-Oxo-2-(1,3-thiazolidin-3-yl)ethyl]amino}-3-azabicyclo[3.1.0]hex-3-yl]nicotinonitrile (Compound No. 110)

ESI-MS (m/z): 330.1 (M⁺+1)
N-(4-Fluorophenyl)-6-{[2-oxo-2-(1,3-thiazolidin-3-yl)ethyl]amino}-3-azabicyclo[3.1.0]hexane-3-carboxamide (Compound No. 111)

ESI-MS (m/z): 365.1 (M⁺+1)
(2S)-1-{N-[3-(4-Chlorobenzoyl)-3-azabicyclo[3.1.0]hex-6-yl]glycyl}pyrrolidine-2-carbonitrile (Compound No. 112)

ESI-MS (m/z): 373.1 (M⁺+1)
(2S)-1-(N-{3-[(Benzyloxy)acetyl]-3-azabicyclo[3.1.0]hex-6-yl}glycyl)pyrrolidine-2-carbonitrile (Compound No. 113)

ESI-MS (m/z): 383.2 (M⁺+1)
(2S)-1-{N-[3-(3,4-Dichlorobenzoyl)-3-azabicyclo[3.1.0]hex-6-yl]glycyl}pyrrolidine-2-carbonitrile (Compound No. 114)

ESI-MS (m/z): 407.1 (M⁺+1)
(2S)-1-{N-[3-(2,6-Dimethoxybenzoyl)-3-azabicyclo[3.1.0]hex-6-yl]glycyl}pyrrolidine-2-carbonitrile (Compound No. 115)

ESI-MS (m/z): 399.2 (M⁺+1)
(2S)-1-{N-[3-(4-Methoxybenzoyl)-3-azabicyclo[3.1.0]hex-6-yl]glycyl}pyrrolidine-2-carbonitrile (Compound No. 116)

ESI-MS (m/z): 369.1 (M⁺+1)
(2S)-1-(N-{3-[(4-Methoxyphenyl)acetyl]-3-azabicyclo[3.1.0]hex-6-yl}glycyl)pyrrolidine-2-carbonitrile (Compound No. 117)

ESI-MS (m/z): 384.2 (M⁺+1)
(2S)-1-{N-[3-(4-tert-Butylbenzoyl)-3-azabicyclo[3.1.0]hex-6-yl]glycyl}pyrrolidine-2-carbonitrile (Compound No. 118)

ESI-MS (m/z): 395.2 (M⁺+1)
(2S)-1-{N-[3-(2,4-Dichlorobenzoyl)-3-azabicyclo[3.1.0]hex-6-yl]glycyl}pyrrolidine-2-carbonitrile (Compound No. 119)

ESI-MS (m/z): 407.1 (M⁺+1)
(2S)-1-{N-[3-(4-Cyanobenzoyl)-3-azabicyclo[3.1.0]hex-6-yl]glycyl}pyrrolidine-2-carbonitrile (Compound No. 120)

ESI-MS (m/z): 364.1 (M⁺+1)
((2S)-1-{N-[3-(3-Chlorobenzoyl)-3-azabicyclo[3.1.0]hex-6-yl]glycyl}pyrrolidine-2-carbonitrile (Compound No. 121)

ESI-MS (m/z): 373.1 (M⁺+1)
(2S)-1-(N-{3-[4-(Trifluoromethoxy)benzoyl]-3-azabicyclo[3.1.0]hex-6-yl}glycyl)pyrrolidine-2-carbonitrile (Compound No. 122),

ESI-MS (m/z): 423.1 (M⁺+1)
(2S)-1-{N-[3-(2,6-Difluorobenzoyl)-3-azabicyclo[3.1.0]hex-6-yl]glycyl}pyrrolidine-2-carbonitrile (Compound No. 123),

ESI-MS (m/z): 375.10 (M⁺+1)
(2S)-1-{N-[3-(4-Methylbenzoyl)-3-azabicyclo[3.1.0]hex-6-yl]glycyl}pyrrolidine-2-carbonitrile (Compound No. 124),

ESI-MS (m/z): 353.21
(2S,4S)—N-{4-[6-({2-[2-Cyano-4-fluoropyrrolidin-1-yl]-2-oxoethyl}amino)-3-azabicyclo[3.1.0]hex-3-yl]phenyl}methanesulphonamide (Compound No. 125)

ESI-MS (m/z): 422.23
(2S,4S)—N-{4-[6-({2-[2-Cyano-4-fluoropyrrolidin-1-yl]-2-oxoethyl}amino)-3-azabicyclo[3.1.0]hex-3-yl]phenyl}-4-methylbenzenesulphonamide (Compound No. 126),

ESI-MS (m/z): 498.30

### Example 11

### Synthesis of (2S)—N-[2-[2-cyanopyrrolidin-1-yl]-2-oxoethyl]-N-[3-(4-fluorobenzoyl)-3-azabicyclo[3.1.0]hex-6-yl]glycinamide (Compound No. 25)

### Step a: Synthesis of tert-butyl (2-{[3-(4-fluorobenzoyl)-3-azabicyclo[3.1.0]hex-6-yl]amino}-2-oxoethyl)carbamate

To a solution of 3-(4-fluorobenzoyl)-3-azabicyclo[3.1.0]hexan-6-ammonium p-toluenesulphonate (1.0 g, 2.55 mmol) in tetrahydrofuran (20.0 mL) at 0° C., was added a solution of N-(tert-butyloxycarbonyl)glycine (0.446 g, 2.55 mmol), triethylamine (0.4 mL, 2.80 mmol) and 1-hydroxbenzotriazole (0.391 g, 2.55 mmol) in tetrahydrofuran followed by a solution of N,N-dicyclohexylcarbodiimide (0.580 g, 2.80 mmol) in dry dichloromethane. The reaction mixture was stirred at 0° C. for about 1 hour followed by overnight at room temperature. The precipitate was filtered and the filtrate diluted with ethylacetate, washed with water, aqueous citric acid (10%), water, aqueous sodium bicarbonate (10%), water and brine. The organic layer was dried over anhydrous sodium sulphate, and concentrated under reduced pressure. The residue, obtained, was purified by column chromatography using 10% methanol in dichloromethane as eluant (silica gel 100-200 mesh) to yield the title compound (610.0 mg, 63%).

### Step b: Synthesis of N-[3-(4-fluorobenzoyl)-3-azabicyclo[3.1.0]hex-6-yl]glycinamide (pTSA salt)

To the compound (610 mg, 1.62 mmol) obtained from step a in acetonitrile (10.0 mL), was added p-toluenesulphonic acid (0.474 g, 2.4 mmol). The mixture was stirred for about 24 hours at room temperature. The solvent was evaporated and the residue taken in ethyl acetate and cooled to 0° C. The precipitate was filtered, washed with cold ethyl acetate and dried to yield the title compound as colourless solid (0.620 g, 54%).

¹H NMR (400 MHz, MeOH-d₄): δ 1.77-1.84 (m, 2H), 2.36 (s, 3H), 2.40 (s, 1H), 3.55-3.75 (m, 5H), 4.15 (d, 1H, J=12.0 Hz), 7.16-7.24 (m, 4H), 7.51-7.54 (m, 2H), 7.70 (d, 2H, J=8.0 Hz).

### Step c: Synthesis of (2S)—N-{2-[2-cyanopyrrolidin-1-yl]-2-oxoethyl}-N′-[3-(4-fluorobenzoyl)-3-azabicyclo[3.1.0]hex-6-yl]glycinamide

To a solution of compound (234 mg, 0.52 mmol) obtained from step b in anhydrous dimethylformamide (2.0 mL) under N₂ atmosphere at room temperature, was added potassium carbonate (100 mg) and a solution of (2S)-1-(chloroacetyl)pyrrolidine-2-carbonitrile (75 mg, 0.43 mmol) in dimethylformamide (2.0 mL). The resulting mixture was stirred overnight and partitioned between dichloromethane (15.0 mL) and water (15.0 mL). The organic layer was washed with water and brine, dried over anhydrous sodium sulphate and concentrated under reduced pressure. The residue was purified by column chromatography using 2% methanol in dichloromethane as eluant (silica gel 100-200 mesh) to yield the title compound (10 mg, 6%).

¹H NMR (400 MHz, CDCl₃): δ 1.3-1.5 (m, 2H), 2.1-2.40 (m, 4H), 2.49 (s, 1H), 3.15-3.45 (m, 4H), 3.45-3.8 (m, 5H), 4.26 (d, 0.8H, J=8.0 Hz, CHCN, rotomer), 4.5 (d, 0.2H, J=8.0 Hz, CHCN, rotomer), 7.07-7.10 (m, 2H), 7.43-7.52 (m, 2H); ESI-MS (m/z): 414.1 (M⁺+1)

The following illustrative compounds were prepared as per the procedure for (2S)—N²-{2-[2-cyanopyrrolidin-1-yl]-2-oxoethyl}-N-[3-(4-fluorobenzoyl)-3-azabicyclo[3.1.0]hex-6-yl]glycinamide by coupling appropriate amines (instead of N-[3-(4-fluorobenzoyl)-3-azabicyclo[3.1.0]hex-6-yl]glycinamide) with (2S)-1-(chloroacetyl)pyrrolidine-2-carbonitrile or its derivative:
(2S)—N-{2-[2-Cyanopyrrolidin-1-yl]-2-oxoethyl-N-[3-(4-fluorobenzoyl)-3-azabicyclo[3.1.0]hex-6-yl]-L-valinamide (Compound No. 26),

ESI-MS (m/z): 456.1 (M⁺+1);
(2S)—N-{2-[2-Cyanopyrrolidin-1-yl]-2-oxoethyl-N-[3-(4-fluorobenzoyl)-3-azabicyclo[3.1.0]hex-6-yl]-L-phenyalaninamide (Compound No. 27),

ESI-MS (m/z): 504 (M⁺+1).

### Example 12

### Synthesis of (2S,4S, 5S)-1-{N-[3-(4-Methylbenzoyl)-3-azabicyclo[3.1.0]hex-6-yl]glycyl}-4,5-methanoppyrrolidine-2-carbonitrile (Compound No. 127)

### Step a: Synthesis of (2S,4S,5S)-1-(2-chloroacetyl)-4,5-methanoproline-2-carbonitrile

To an ice-cold solution of (2S,4S,5S)-4,5-methanopyrrolidine-3-carbonitrile [pTSA salt] (500 mg, 1.78 mmol) and triethylamine (1.80 g, 17.7 mmol) in anhydrous dichloromethane (10.0 ml), was added a solution of 2-chloroacetyl chloride (605 mg, 5.35 mmol) in anhydrous dichloromethane (10.0 ml) dropwise over 10 minutes. The reaction mixture was stirred overnight at room temperature; diluted with dichloromethane (25 ml) and washed with water, brine and dried over anhydrous sodium sulphate and concentrated under vacuum to obtain the crude product that was purified by column chromatography using 2% methanol in dichloromethane as eluant (silica gel 100-200 mesh) to yield the title compound. (210 mg, 64%).

¹H NMR (400 MHz, CDCl₃): δ 1.04-1.08 (m, 1H), 1.13-1.16 (m, 1H), 1.9-1.94 (m, 1H), 2.40-2.44 (dd, 1H, 2 Hz, 11.5 Hz), 2.55-2.65 (m, 1H), 3.58-3.62 (m, 1H), 4.14-4.25 (m, 2H), 4.94-4.97 (dd, 1H, 2 Hz, 8.4 Hz); ESI-MS (m/z): 185.7 (M⁺+1).

### Step b: Synthesis of (2S,4S,5S)-1-(N-{3-[(4-fluorophenyl)carbonyl]-3-azabicyclo[3.1.0]hex-6-yl}glycyl)-4,5-methanoproline-2-carbonitrile

To a solution of 3-[4-fluorobenzoyl]-3-azabicyclo[3.1.0]hexan-6-amine (pTSA salt) (189 mg, 0.48 mmol) in anhydrous dimethylformamide (2.0 mL) under N₂ atmosphere at room temperature, was added potassium carbonate (215 mg, 1.55 mmol) and stirred for about 15 minutes. A solution of potassium iodide (44 mg, 0.26 mmol) in anhydrous dimethylformamide (1.0 mL) was added followed by a solution of compound (100 mg, 0.54 mmol) in anhydrous dimethylformamide (0.5 mL) obtained from step a. The reaction mixture was stirred overnight and partitioned between water and ethyl acetate. The organic layer was washed with water and brine, dried over anhydrous sodium sulphate and concentrated under reduced pressure. The residue was purified by column chromatography using 5% methanol in dichloromethane (silica gel 100-200 mesh) as eluant to yield the title compound (70 mg, 35%).

¹H NMR (400 MHz, CD₃OD): δ 0.8-0.9 (m, 1H), 0.9-1.2 (m, 1H), 1.70-1.75 (m, 2H), 1.88-1.96 (bt, 1H), 2.03-2.05 (m, 1H), 2.28-2.31 (dd, 1H, 2 Hz, 12.7 Hz), 2.55-2.7 (br, 1H), 3.44-3.52 (m, 2H), 3.60-3.72 (m, 4H), 4.00-4.04 (dd, 1H, 4.9 Hz, 7.3 Hz), 5.01-5.03 (dd, 1H, 2 Hz, 10.4 Hz), 7.14-7.18 (t, 2H, 8.6 Hz), 7.47-7.49 (br, 2H); ESI-MS (m/z): 369 (M⁺+1).

### DPP IV Assay

### Materials:

H-Gly-Pro-7-amido-methylcoumarine (Gly-Pro-AMC; Cat. # G2761) and coumarine (AMC; Cat. # A9891) were purchased from Sigma. A stock solution of 1 mM Gly-Pro-AMC was prepared in 50 mM HEPES buffer, pH 7.8, containing 80 mM MgCl₂, 140 mM NaCl and 1% BSA (working buffer). A solution of 1 mM AMC was prepared in 10% dimethylsulfoxide (DMSO). Aliquots were stored at −20° C.

### DPP IV Assay:

The DPP IV enzyme activity was determined using the fluorometric assay with the substrate Gly-Pro-AMC, which is cleaved by DPP IV to release the fluorescent AMC leaving group. The test compounds were dissolved in 100% dimethylsulfoxide to get a final concentration of 10 mM. The compounds were diluted serially in 10% DMSO to get 10× concentrations of 10 nM, 100 nM, 1000 nM, 10 μM, 100 μM, and 1000 μM. The source of DPP IV was human plasma, which was procured from local blood bank. DPP IV (10 μl human plasma) was mixed in 96-well FluoroNunc plates with test compounds. The final concentrations of the compounds were 1 nM, 10 nM, 100 nM, 1000 μM, 10 μM and 100 μM in working buffer, which were pre-incubated at 25° C. for 15 min. The assay was also carried out with 1% DMSO (final concentration), lacking the compound, as vehicle control. The reaction was started by adding 20 μl of 0.1 mM H-Gly-Pro-AMC (40 μM final concentration), followed by mixing and incubation at 25° C. for 20 min. The reaction was arrested by adding 50 μl of 25% acetic acid. The fluorescence was measured at an excitation filter of 380 nM and emission filter of 460 nM.

The DPP IV releases AMC from Gly-Pro-AMC, which was quantitated as relative fluorescence units (RFU). The percentage of activity was calculated as follows:

=(100/RFU of vehicle control)×RFU of test (with compound)

### IC₅₀ Determination

The IC₅₀ is defined as the concentration of the inhibitor required to inhibit 50% of the human DPP IV activity under specific assay conditions. The activity obtained at different concentrations of the compound was plotted as log (X) vs. % activity in y-axis. The IC₅₀ values were calculated using non-linear regression analysis (GradPad Prism4).

Compounds specifically disclosed herein displaed IC₅₀ for the DPP IV assay in a range from about 26 μM to more than 100 μM, or from about 26 μM to about 1000 μM, or from out 26 μM to about 600 μM, or from about 26 μM to about 300 μM, or from about 26 μM to about 140 μM, or from about 26 μM to about 80 μM.

We claim:

1. Compounds having the structure of Formula I: 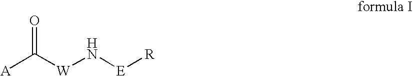
including pharmaceutically acceptable salts, pharmaceutically acceptable solvates, enantiomers, diastereomers, polymorphs, prodrugs, metabolites or N-oxides thereof,
wherein
A is a group selected from 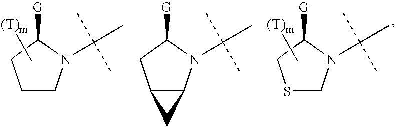
wherein
G is selected from H, —CN, —COR₁, —CR₂═NOH, —CR₂═NR₂ or B(R₃)(R₄) (wherein R₁ is hydrogen, CF₃, alkyl, aryl or heteroaryl; R₂ is H, alkyl, aryl or heteroaryl; R₃ and R₄ are independently selected from —OH or —OR₅ [wherein —OR₅ can be hydrolysed to —OH and R₅ is alkyl, cycloalkyl or aryl]; If R₃ and R₄ are OR₅, then R₃ and R₄ may together form a ring of 5 to 8 atoms);
T is cyano, halogen, alkyl, alkenyl, alkynyl, hydroxy, alkoxy, carbonyl, thiocarbonyl, and oxo and n is an integer of 0 to 3.
W is —C(R^{x}R_{y})ₙ—, wherein n is an integer of 1 to 3 and Rₓ and R_{y} are independently selected from the group consisting of hydrogen, alkyl, alkenyl, alkynyl, cycloalkyl, aryl, heteroaryl or heterocyclyl;
E is —(CRₓR_{y})ₘ— or —C(RₓR_{y})CON(Rₓ)—(wherein m is an integer of 0 to 3 and Rₓ and R_{y} are defined as above), when E=—(CRₓR_{y})ₘ— and m=0 to 1 then R can be 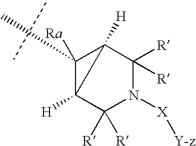
when E=—(CRₓR_{y})ₘ— or —C(RₓR_{y})CON(Rₓ)— and m=2-3, then R comprises one of the following Formulas: 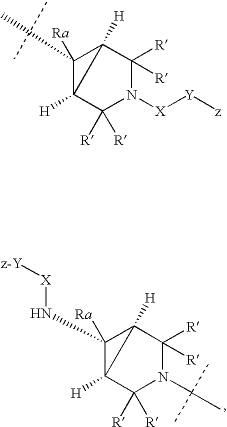
wherein
X is no atom, —CO—, —CS—, —C(RₓR_{y})₁ and —SO₂—, wherein 1 is an integer of 1-3; and Rₓ and R_{y} are defined as above;
Y is no atom, —O— or —NRₓ— with the proviso that Y cannot be —O— or —NRₓ— when X═C(RₓR_{y})₁ [wherein 1 is 1];
Z is alkyl, alkenyl, alkynyl, cycloalkyl, aryl, heteroaryl or heterocyclyl;
Rₐ is hydrogen, alkyl or aryl; and
R′ is Rₓ, —CN, carboxy, halogen, carbonyl, or amino.

2. A compound as claimed in claim 1 of general Formula Ia, 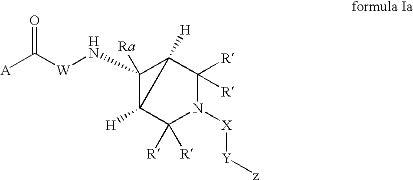
including pharmaceutically acceptable salts, pharmaceutically acceptable solvates, enantiomers, diastereomers, polymorphs, prodrugs, metabolites or N-oxides thereof,
wherein
A is a group selected from 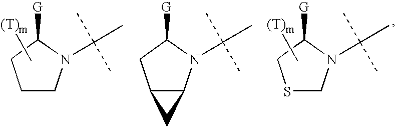
wherein
G is selected from H, —CN, —COR₁, —CR₂═NOH, —CR₂═NR₂ or B(R₃)(R₄) (wherein R₁ is hydrogen, CF₃, alkyl, aryl or heteroaryl; R₂ is H, alkyl, aryl or heteroaryl; R₃ and R₄ are independently selected from —OH or —OR₅ [wherein —OR₅ can be hydrolysed to —OH and R₅ is alkyl, cycloalkyl or aryl]; If R₃ and R₄ are OR₅, then R₃ and R₄ may together form a ring of 5 to 8 atoms);
T is cyano, halogen, alkyl, alkenyl, alkynyl, hydroxy, alkoxy, carbonyl, thiocarbonyl, and oxo.
W is —C(RₓR_{y})ₙ—, wherein n is an integer of 1 to 3 and Rₓ and R_{y} are independently selected from the group consisting of hydrogen, alkyl, alkenyl, alkynyl, cycloalkyl, aryl, heteroaryl or heterocyclyl;
X is no atom, —CO—, —CS—, —C(RₓR_{y})ₙ— and —SO₂—, wherein 1 is an integer of 1-3; and
Rₓ and R_{y} are defined as above;
Y is no atom, —O— or —NR_{c}— with the proviso that Y cannot be —O— or —NRₓ— when X═C(RₓR_{y})₁ [wherein 1 is 1];
Z is alkyl, alkenyl, alkynyl, cycloalkyl, aryl, heteroaryl or heterocyclyl;
Rₐ is hydrogen, alkyl or aryl; and
R′ is Rₓ, —CN, carboxy, halogen, carbonyl, or amino.

3. A compound as claimed in claim 1 of general Formula Ib, 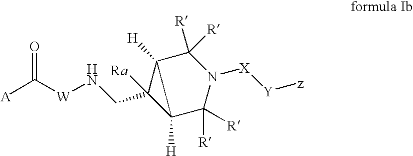
including pharmaceutically acceptable salts, pharmaceutically acceptable solvates, enantiomers, diastereomers, polymorphs, prodrugs, metabolites or N-oxides thereof,
wherein
A is a group selected from 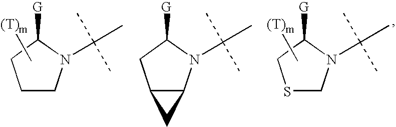
wherein
G is selected from H, —CN, —COR₁, —CR₂—NOH, —CR₂═NR₂ or B(R₃)(R₄) (wherein R₁ is hydrogen, CF₃, alkyl, aryl or heteroaryl; R₂ is H, alkyl, aryl or heteroaryl; R₃ and R₄ are independently selected from —OH or —OR₅ [wherein —OR₅ can be hydrolysed to —OH and R₅ is alkyl, cycloalkyl or aryl]; If R₃ and R₄ are OR₅, then R₃ and R₄ may together form a ring of 5 to 8 atoms);
T is cyano, halogen, alkyl, alkenyl, alkynyl, hydroxy, alkoxy, carbonyl, thiocarbonyl, and oxo.
W is —C(RₓR_{y})ₙ— wherein n is an integer of 1 to 3 and Rₓ and R_{y} are independently selected from the group consisting of hydrogen, alkyl, alkenyl, alkynyl, cycloalkyl, aryl, heteroaryl or heterocyclyl;
X is no atom, —CO—, —CS—, —C(RₓR_{y})₁— and —SO₂—, wherein 1 is an integer of 1-3; and
Rₓ and R_{y} are defined as above;
Y is no atom, —O— or —NR_{c}— with the proviso that Y cannot be —O— or —NR_{c}— when X═C(RₓR_{y})₁ [wherein 1 is 1];
Z is alkyl, alkenyl, alkynyl, cycloalkyl, aryl, heteroaryl or heterocyclyl;
Rₐ is hydrogen, alkyl or aryl; and
R′ is Rₓ, —CN, carboxy, halogen, carbonyl, or amino.

4. A compound as claimed in claim 1 of general Formula Ic, 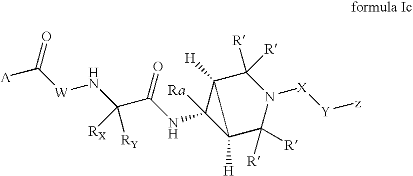
including pharmaceutically acceptable salts, pharmaceutically acceptable solvates, enantiomers, diastereomers, polymorphs, prodrugs, metabolites or N-oxides thereof,
wherein
A is a group selected from 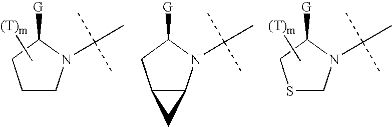
G is selected from H, —CN, —COR₁, —CR₂═NOH, —CR₂═NR₂ or B(R₃)(R₄) (wherein R₁ is hydrogen, CF₃, alkyl, aryl or heteroaryl; R₂ is H, alkyl, aryl or heteroaryl; R₃ and R₄ are independently selected from —OH or —OR₅ [wherein —OR₅ can be hydrolysed to —OH and R₅ is alkyl, cycloalkyl or aryl]; If R₃ and R₄ are OR₅, then R₃ and R₄ may together form a ring of 5 to 8 atoms);
T is cyano, halogen, alkyl, alkenyl, alkynyl, hydroxy, alkoxy, carbonyl, thiocarbonyl, and oxo. W is —C(RₓR_{y})ₙ— wherein n is an integer of 1 to 3 and Rₓ and R_{y} are independently selected from the group consisting of hydrogen, alkyl, alkenyl, alkynyl, cycloalkyl, aryl, heteroaryl or heterocyclyl;
X is no atom, —CO—, —CS—, —C(RₓR_{y})₁— and —SO₂—, wherein 1 is an integer of 1-3; and
Rₓ and R_{y} are defined as above;
Y is no atom, —O— or —NRₓ— with the proviso that Y cannot be —O— or —NRₓ— when X═C(RₓR_{y})₁ [wherein 1 is 1];
Z is alkyl, alkenyl, alkynyl, cycloalkyl, aryl, heteroaryl or heterocyclyl;
Rₐ is hydrogen, alkyl or aryl; and
R′ is Rₓ, —CN, carboxy, halogen, carbonyl, or amino.

15. Compounds selected from the group consisting of:
(2S)-1-(N-{3-[(4-Methylphenyl)sulphonyl]-3-azabicyclo[3.1.0]hex-6-yl}glycyl)pyrrolidine-2-carbonitrile (Compound No. 01),
(2S)-1-{N-[3-(4-Fluorobenzoyl)-3-azabicyclo[3.1.0]hex-6-yl]glycyl}pyrrolidine-2-carbonitrile (Compound No. 02),
(2S)-1-[N-({3-[(4-Fluorophenyl)sulphonyl]-3-azabicyclo[3.1.0]hex-6-yl}methyl)glycyl]pyrrolidine-2-carbonitrile (Compound No. 03).
(2S)-1-[N-(3-Benzyl-3-azabicyclo[3.1.0]hex-6-yl)glycyl]pyrrolidine-2-carbonitrile (Compound No. 04),
Phenyl (2S)-6-{[2-(2-cyano-4,4-difluoropyrrolidin-1-yl)-2-oxoethyl]amino}-3-azabicyclo[3.1.0]hexane-3-carboxylate (Compound No. 05),
(2S)-4,4-Difluoro-1-(N-{3-[(4-fluorophenyl)sulphonyl]-3-azabicyclo[3.1.0]hex-6-yl}glycyl)pyrrolidine-2-carbonitrile (Compound No. 06),
4-Chlorophenyl (2S)-6-{[2-(2-cyano-4,4-difluoropyrrolidin-1-yl)-2-oxoethyl]amino}-3-azabicyclo[3.1.0]hexane-3-carboxylate (Compound No. 07),
Phenyl (2S,4S)-6-({2-[2-cyano-4-fluoropyrrolidin-1-yl]-2-oxoethyl}amino)-3-azabicyclo[3.1.0]hexane-3-carboxylate (Compound No. 08),
(2S)-1-(N-{3-[(4-Fluorophenyl)sulphonyl]-3-azabicyclo[3.1.0]hex-6-yl}glycyl)pyrrolidine-2-carbonitrile (Compound No. 09),
(2S)-1-[N-(3-Benzyl-3-azabicyclo[3.1.0]hex-6-yl)glycyl]-4,4-difluoropyrrolidine-2-carbonitrile (Compound No. 10),
(2S,4S)-4-Fluoro-1-(N-{3-[(4-fluorophenyl)sulphonyl]-3-azabicyclo[3.1.0]hex-6-yl}glycyl)pyrrolidine-2-carbonitrile (Compound No. 11),
(2S)-4-Chlorophenyl 6-{[2-(2-cyanopyrrolidin-1-yl)-2-oxoethyl]amino}-3-azabicyclo[3.1.0]hexane-3-carboxylate (Compound No. 12)
(2S,4S)-1-(N-{3-[(4-tert-Butylphenyl)sulphonyl]-3-azabicyclo[3.1.0]hex-6-yl}glycyl)-4-fluoropyrrolidine-2-carbonitrile (Compound No. 13)
(2S,4S)-4-Fluoro-1-{N-[3-(4-fluorobenzoyl)-3-azabicyclo[3.1.0]hex-6-yl]glycyl}pyrrolidine-2-carbonitrile (Compound No. 14),
(2S)-4,4-Difluoro-1-(N-{3-[5-(trifluoromethyl)pyridin-2-yl]-3-azabicyclo[3.1.0]hex-6-yl}glycyl)pyrrolidine-2-carbonitrile (Compound No. 15),
(2S)-6-{[2-(2-Cyano-4,4-difluoropyrrolidin-1-yl)-2-oxoethyl]amino}-N-(4-fluorophenyl)-3-azabicyclo[3.1.0]hexane-3-carboxamide (Compound No. 16),
(2S)-4,4-Difluoro-1-{N-[3-(4-fluorobenzoyl)-3-azabicyclo[3.1.0]hex-6-yl]glycyl}pyrrolidine-2-carbonitrile (Compound No. 17),
(2S)-1-{N-[3-(2-Naphthoyl)-3-azabicyclo[3.1.0]hex-6-yl]glycyl}pyrrolidine-2-carbonitrile (Compound No. 18),
(2S)-1-{N-[3-(Quinolin-2-ylcarbonyl)-3-azabicyclo[3.1.0]hex-6-yl]glycyl}pyrrolidine-2-carbonitrile (Compound No. 19),
(2S)-1-{N-[3-(1-Adamantylcarbonyl)-3-azabicyclo[3.1.0]hex-6-yl]glycyl}pyrrolidine-2-carbonitrile (Compound No. 20),
Phenyl (2S,4S)-6-({2-[2-cyano-4-fluoropyrrolidin-1-yl]-2-oxoethyl}amino)-3-azabicyclo[3.1.0]hexane-3-carboxylate (Compound No. 21),
(2S,4S)-6-({2-[2-Cyano-4-fluoropyrrolidin-1-yl]-2-oxoethyl}amino)-N-(4-cyanophenyl)-3-azabicyclo[3.1.0]hexane-3-carboxamide (Compound No. 22),
(2S)-6-(6-{[2-(2-Cyano-4,4-difluoropyrrolidin-1-yl)-2-oxoethyl]amino}-3-azabicyclo[3.1.0]hex-3-yl)nicotinonitrile (Compound No. 23),
(2S,4S)-6-[6-({2-[2-Cyano-4-fluoropyrrolidin-1-yl]-2-oxoethyl}amino)-3-azabicyclo[3.1.0]hex-3-yl]nicotinonitrile (Compound No. 24)
(2S)—N-{2-[2-Cyanopyrrolidin-1-yl]-2-oxoethyl-N′-[3-(4-fluorobenzoyl)-3-azabicyclo[3.1.0]hex-6-yl]glycinamide (Compound No. 25)
(2S)—N-{2-[2-Cyanopyrrolidin-1-yl]-2-oxoethyl-N′-[3-(4-fluorobenzoyl)-3-azabicyclo[3.1.0]hex-6-yl]-L-valinamide (Compound No. 26)
(2S)—N-{2-[2-Cyanopyrrolidin-1-yl]-2-oxoethyl-N-[3-(4-fluorobenzoyl)-3-azabicyclo[3.1.0]hex-6-yl]-L-phenyalaninamide (Compound No. 27)
(2S,4S)—N-(4-{[6-({2-[2-Cyano-4-fluoropyrrolidin-1-yl]-2-oxoethyl}amino)-3-azabicyclo[3.1.0]hex-3-yl]carbonyl}phenyl)-4-methylbenzenesulphonamide (Compound No. 28)
(2S,4S)—N-(4-{[6-({2-[2-Cyano-4-fluoropyrrolidin-1-yl]-2-oxoethyl}amino)-3-azabicyclo[3.1.0]hex-3-yl]carbonyl}phenyl)methanesulphonamide (Compound No. 29)
(2S,4S)—N-(4-{[6-({2-[2-Cyano-4-fluoropyrrolidin-1-yl]-2-oxoethyl}amino)-3-azabicyclo[3.1.0]hex-3-yl]carbonyl}phenyl)-4-fluorobenzenesulphonamide (Compound No. 30)
(2S,4S)-1-(4-{[6-({2-[2-Cyano-4-fluoropyrrolidin-1-yl]-2-oxoethyl}amino)-3-azabicyclo[3.1.0]hex-3-yl]carbonyl}phenyl)-3-(4-fluorophenyl)urea (Compound No. 31)
(2S,4S)—N-(4-{[6-({2-[2-Cyano-4-fluoropyrrolidin-1-yl]-2-oxoethyl}amino)-3-azabicyclo[3.1.0]hex-3-yl]carbonyl}phenyl)-4-fluorobenzamide (Compound No. 32)
(2S,4S)-4-[6-({2-[2-Cyano-4-fluoropyrrolidin-1-yl]-2-oxoethyl}amino)-3-azabicyclo[3.1.0]hex-3-yl]benzamide (Compound No. 33)
(2S,4S)-4-[6-({2-[2-Cyano-4-fluoropyrrolidin-1-yl]-2-oxoethyl}amino)-3-azabicyclo[3.1.0]hex-3-yl]-N-cyclopropylbenzamide (Compound No. 34)
(2S,4S)-4-[6-({2-[2-Cyano-4-fluoropyrrolidin-1-yl]-2-oxoethyl}amino)-3-azabicyclo[3.1.0]hex-3-yl]-N-methylbenzamide (Compound No. 35)
(2S,4S)-1-(N-{3-[4-(Dimethylamino)benzoyl]-3-azabicyclo[3.1.0]hex-6-yl}glycyl)-4-fluoropyrrolidine-2-carbonitrile (Compound No. 36)
(2S,4S)—N-(4-{[6-({2-[2-Cyano-4-fluoropyrrolidin-1-yl]-2-oxoethyl}amino)-3-azabicyclo[3.1.0]hex-3-yl]carbonyl}phenyl)acetamide (Compound No. 37)
(2S,4S)-1-{N-[3-(4-Acetylbenzoyl)-3-azabicyclo[3.1.0]hex-6-yl]glycyl}-4-fluoropyrrolidine-2-carbonitrile (Compound No. 38)
(2S,4S)-4-Fluoro-1-(N-{3-[4-(trifluoromethyl)benzoyl]-3-azabicyclo[3.1.0]hex-6-yl}glycyl)pyrrolidine-2-carbonitrile (Compound No. 39)
(2S,4S)-1-{N-[3-(4-Acetylphenyl)-3-azabicyclo[3.1.0]hex-6-yl]glycyl}-4-fluoropyrrolidine-2-carbonitrile (Compound No. 40)
Ethyl (2S,4S)-4-[6-({2-[2-Cyano-4-fluoropyrrolidin-1-yl]-2-oxoethyl}amino)-3-azabicyclo[3.1.0]hex-3-yl]benzoate (Compound No. 41)
(2S,4S)-4-Fluoro-1-(N-{3-[4-(trifluoromethoxy)benzoyl]-3-azabicyclo[3.1.0]hex-6-yl}glycyl)pyrrolidine-2-carbonitrile (Compound No. 42)
(2S,4S)-1-{N-[3-(4-tert-Butylbenzoyl)-3-azabicyclo[3.1.0]hex-6-yl]glycyl}-4-fluoropyrrolidine-2-carbonitrile (Compound No. 43)
(2S,4S)-4-Fluoro-1-{N-[3-(4-methoxybenzoyl)-3-azabicyclo[3.1.0]hex-6-yl]glycyl}pyrrolidine-2-carbonitrile (Compound No. 44)
(2S,4S)-4-Fluoro-1-{N-[3-(4-methylbenzoyl)-3-azabicyclo[3.1.0]hex-6-yl]glycyl}pyrrolidine-2-carbonitrile (Compound No. 45)
(2S,4S)-4-Fluoro-1-{N-[3-phenyl-3-azabicyclo[3.1.0]hex-6-yl]glycyl}pyrrolidine-2-carbonitrile (Compound No. 46)
(2S,4S)-1-{N-[3-(4-Cyano-1-naphthyl)-3-azabicyclo[3.1.0]hex-6-yl]glycyl}-4-fluoropyrrolidine-2-carbonitrile (Compound No. 47)
(2S,4S)-1-(N-{3-[4-Cyano-3-(trifluoromethyl)phenyl]-3-azabicyclo[3.1.0]hex-6-yl}glycyl)-4-fluoropyrrolidine-2-carbonitrile (Compound No. 48)
(2S,4S)-1-(N-{3-[2-Cyano-3-(trifluoromethyl)phenyl]-3-azabicyclo[3.1.0]hex-6-yl}glycyl)-4-fluoropyrrolidine-2-carbonitrile (Compound No. 49)
(2S,4S)-1-{N-[3-(3-Chloro-2-cyanophenyl)-3-azabicyclo[3.1.0]hex-6-yl]glycyl}-4-fluoropyrrolidine-2-carbonitrile (Compound No. 50)
(2S,4S)-1-(N-{3-[2-Cyano-4-(trifluoromethyl)phenyl]-3-azabicyclo[3.1.0]hex-6-yl}glycyl)-4-fluoropyrrolidine-2-carbonitrile (Compound No. 51)
(2S,4S)-1-{N-[3-(4-Cyano-2-fluorophenyl)-3-azabicyclo[3.1.0]hex-6-yl]glycyl}-4-fluoropyrrolidine-2-carbonitrile (Compound No. 52)
(2S,4S)-1-{N-[3-(2-Cyano-3-fluorophenyl)-3-azabicyclo[3.1.0]hex-6-yl]glycyl}-4-fluoropyrrolidine-2-carbonitrile (Compound No. 53)
(2S,4S)-1-{N-[3-(2-Cyanophenyl)-3-azabicyclo[3.1.0]hex-6-yl]glycyl-4-fluoropyrrolidine-2-carbonitrile (Compound No. 54)
(2S,4S)-1-(N-{3-[4-Cyano-2-(trifluoromethyl)phenyl]-3-azabicyclo[3.1.0]hex-6-ylglycyl)-4-fluoropyrrolidine-2-carbonitrile (Compound No. 55)
(2S,4S)-1-{N-[3-(4-Cyano-3-fluorophenyl)-3-azabicyclo[3.1.0]hex-6-yl]glycyl}-4-fluoropyrrolidine-2-carbonitrile (Compound No. 56)
(2S,4S)-1-{N-[3-(2-Chloro-4-cyanophenyl)-3-azabicyclo[3.1.0]hex-6-yl]glycyl}-4-fluoropyrrolidine-2-carbonitrile (Compound No. 57)
(2S,4S)-1-{N-[3-(4-Cyanophenyl)-3-azabicyclo[3.1.0]hex-6-yl]glycyl}-4-fluoropyrrolidine-2-carbonitrile (Compound No. 58)
(2S,4S)-1-{N-[3-(3-Chloro-4-cyanophenyl)-3-azabicyclo[3.1.0]hex-6-yl]glycyl}-4-fluoropyrrolidine-2-carbonitrile (Compound No. 59)
(2S,4S)-1-{N-[3-(2-Cyano-6-fluorophenyl)-3-azabicyclo[3.1.0]hex-6-yl]glycyl}-4-fluoropyrrolidine-2-carbonitrile (Compound No. 60)
(2S,4S)-1-{N-[3-(2-Cyano-4-fluorophenyl)-3-azabicyclo[3.1.0]hex-6-yl]glycyl}-4-fluoropyrrolidine-2-carbonitrile (Compound No. 61)
(2S,4S)-1-{N-[3-(4-Cyanobenzoyl)-3-azabicyclo[3.1.0]hex-6-yl]glycyl}-4-fluoropyrrolidine-2-carbonitrile (Compound No. 62)
(2S,4S)-4-Fluoro-1-(N-{3-[2-fluoro-3-(trifluoromethyl)benzoyl]-3-azabicyclo[3.1.0]hex-6-yl}glycyl)pyrrolidine-2-carbonitrile (Compound No. 63)
(2S,4S)-4-Fluoro-1-(N-{3-[2-fluoro-5-(trifluoromethyl)benzoyl]-3-azabicyclo[3.1.0]hex-6-yl}glycyl)pyrrolidine-2-carbonitrile (Compound No. 64)
(2S,4S)-4-Fluoro-1-(N-{3-[4-fluoro-3-(trifluoromethyl)benzoyl]-3-azabicyclo[3.1.0]hex-6-yl}glycyl)pyrrolidine-2-carbonitrile (Compound No. 65)
(2S,4S)-4-Fluoro-1-(N-{3-[4-fluoro-2-(trifluoromethyl)benzoyl]-3-azabicyclo[3.1.0]hex-6-yl}glycyl)pyrrolidine-2-carbonitrile (Compound No. 66)
(2S,4S)-4-Fluoro-1-(N-{3-[3-fluoro-4-(trifluoromethyl)benzoyl]-3-azabicyclo[3.1.0]hex-6-yl}glycyl)pyrrolidine-2-carbonitrile (Compound No. 67)
(2S,4S)-1-{N-[3-(3-Chloro-2-fluorobenzoyl)-3-azabicyclo[3.1.0]hex-6-yl]glycyl}-4-fluoropyrrolidine-2-carbonitrile (Compound No. 68)
(2S,4S)-1-(N-{3-[2,4-Bis(trifluoromethyl)benzoyl]-3-azabicyclo[3.1.0]hex-6-yl}glycyl)-4-fluoropyrrolidine-2-carbonitrile (Compound No. 69)
(2S,4S)-1-{N-[3-(2,6-Difluorobenzoyl)-3-azabicyclo[3.1.0]hex-6-yl]glycyl}-4-fluoropyrrolidine-2-carbonitrile (Compound No. 70)
(2S,4S)-1-{N-[3-(2,4-Difluorobenzoyl)-3-azabicyclo[3.1.0]hex-6-yl]glycyl}-4-fluoropyrrolidine-2-carbonitrile (Compound No. 71)
(2S,4S)-1-{N-[3-(3,4-Difluorobenzoyl)-3-azabicyclo[3.1.0]hex-6-yl]glycyl}-4-fluoropyrrolidine-2-carbonitrile (Compound No. 72)
(2S,4S)-1-{N-[3-(3-Chloro-2,4-difluorobenzoyl)-3-azabicyclo[3.1.0]hex-6-yl]glycyl}-4-fluoropyrrolidine-2-carbonitrile (Compound No. 73)
(2S,4S)-1-{N-[3-(2-Chloro-4,5-difluorobenzoyl)-3-azabicyclo[3.1.0]hex-6-yl]glycyl}-4-fluoropyrrolidine-2-carbonitrile (Compound No. 74)
(2S,4S)-4-Fluoro-1-{N-[3-(2,3,4-trifluorobenzoyl)-3-azabicyclo[3.1.0]hex-6-yl]glycyl}pyrrolidine-2-carbonitrile (Compound No. 75)
(2S,4S)-4-Fluoro-1-{N-[3-(3-fluorobenzoyl)-3-azabicyclo[3.1.0]hex-6-yl]glycyl}pyrrolidine-2-carbonitrile (Compound No. 76)
(2S,4S)-4-Fluoro-1-{N-[3-(3-fluorobenzoyl)-3-azabicyclo[3.1.0]hex-6-yl]glycyl}pyrrolidine-2-carbonitrile (Compound No. 77)
(2S,4S)-4-Fluoro-1-{N-[3-(2,4,5-trifluorobenzoyl)-3-azabicyclo[3.1.0]hex-6-yl]glycyl}pyrrolidine-2-carbonitrile (Compound No. 78)
(2S,4S)-1-{N-[3-(3-Chloro-4-fluorobenzoyl)-3-azabicyclo[3.1.0]hex-6-yl]glycyl}-4-fluoropyrrolidine-2-carbonitrile (Compound No. 79)
(2S,4S)-4-Fluoro-1-{N-[3-(4-fluoro-2-methylbenzoyl)-3-azabicyclo[3.1.0]hex-6-yl]glycyl}pyrrolidine-2-carbonitrile (Compound No. 80)
(2S,4S)-4-Fluoro-1-{N-[3-(3,4,5-trifluorobenzoyl)-3-azabicyclo[3.1.0]hex-6-yl]glycyl}pyrrolidine-2-carbonitrile (Compound No. 81)
(2S,4S)-1-{N-[3-(3-Cyano-5-fluorophenyl)-3-azabicyclo[3.1.0]hex-6-yl]glycyl}-4-fluoropyrrolidine-2-carbonitrile (Compound No. 82)
(2S,4S)-1-{N-[3-(2-Cyano-3,5-difluorophenyl)-3-azabicyclo[3.1.0]hex-6-yl]glycyl}-4-fluoropyrrolidine-2-carbonitrile (Compound No. 83)
(2S,4S)-1-{N-[3-(4-Cyano-3,5-difluorophenyl)-3-azabicyclo[3.1.0]hex-6-yl]glycyl}-4-fluoropyrrolidine-2-carbonitrile (Compound No. 84)
(2S,4S)-1-{N-[3-(4-Chlorobenzoyl)-3-azabicyclo[3.1.0]hex-6-yl]glycyl}-4-fluoropyrrolidine-2-carbonitrile (Compound No. 85)
(2S,4S)-1-{N-[3-(5-Chloropyridin-2-yl)-3-azabicyclo[3.1.0]hex-6-yl]glycyl}-4-fluoropyrrolidine-2-carbonitrile (Compound No. 86)
(2S,4S)-4-Fluoro-1-{N-[3-(5-nitropyridin-2-yl)-3-azabicyclo[3.1.0]hex-6-yl]glycyl}pyrrolidine-2-carbonitrile (Compound No. 87)
(2S,4S)-4-Fluoro-1-{N-[3-pyridin-2-yl-3-azabicyclo[3.1.0]hex-6-yl]glycyl}pyrrolidine-2-carbonitrile (Compound No. 88)
(2S,4S)-1-{N-[3-(3-Chloropyridin-2-yl)-3-azabicyclo[3.1.0]hex-6-yl]glycyl}-4-fluoropyrrolidine-2-carbonitrile (Compound No. 89)
(2S,4S)—N-(4-Chlorophenyl)-6-({2-[2-cyano-4-fluoropyrrolidin-1-yl]-2-oxoethyl}amino)-3-azabicyclo[3.1.0]hexane-3-carboxamide (Compound No. 90)
(2S,4S)-4-Fluoro-1-{N-[3-{[4-(trifluoromethyl)phenyl]sulfonyl}-3-azabicyclo[3.1.0]hex-6-yl]glycyl}pyrrolidine-2-carbonitrile (Compound No. 91)
(2S,4S)-1-{N-[3-(1,3-Benzothiazol-2-yl)-3-azabicyclo[3.1.0]hex-6-yl]glycyl}-4-fluoropyrrolidine-2-carbonitrile (Compound No. 92)
(2S,4S)-1-{N-[3-(1,3-Benzoxazol-2-yl)-3-azabicyclo[3.1.0]hex-6-yl]glycyl}-4-fluoropyrrolidine-2-carbonitrile (Compound No. 93)
(2S,4S)-4-Fluoro-1-(N-{3-[4-(trifluoromethyl)pyrimidin-2-yl]-3-azabicyclo[3.1.0]hex-6-yl}glycyl)pyrrolidine-2-carbonitrile (Compound No. 94)
(2S,4S)-4-Fluoro-1-{N-[3-isoquinolin-1-yl-3-azabicyclo[3.1.0]hex-6-yl]glycyl}pyrrolidine-2-carbonitrile (Compound No. 95)
(2S,4S)-4-Fluoro-1-{N-[3-quinoxalin-2-yl-3-azabicyclo[3.1.0]hex-6-yl]glycyl}pyrrolidine-2-carbonitrile (Compound No. 96)
(2S,4S)-4-Fluoro-1-{N-[3-(3-nitropyridin-2-yl)-3-azabicyclo[3.1.0]hex-6-yl]glycyl}pyrrolidine-2-carbonitrile (Compound No. 97)
(2S,4S)-4-Fluoro-1-{N-[3-(1,3-thiazol-2-yl)-3-azabicyclo[3.1.0]hex-6-yl]glycyl}pyrrolidine-2-carbonitrile (Compound No. 98)
(2S,4S)-1-{N-[3-(Cyclohexylcarbonyl)-3-azabicyclo[3.1.0]hex-6-yl]glycyl}-4-fluoropyrrolidine-2-carbonitrile (Compound No. 99)
(2S,4S)-4-Fluoro-1-{N-[3-(methylsulphonyl)-3-azabicyclo[3.1.0]hex-6-yl]glycyl}pyrrolidine-2-carbonitrile (Compound No. 100)
(2S)—N-(4-Chlorophenyl)-6-({2-[2-cyanopyrrolidin-1-yl]-2-oxoethyl}amino)-3-azabicyclo[3.1.0]hexane-3-carboxamide (Compound No. 101)
(2S,4S)-1-(N-{3-[(4-Bromophenyl)sulphonyl]-3-azabicyclo[3.1.0]hex-6-yl}glycyl)-4-fluoropyrrolidine-2-carbonitrile (Compound No. 102)
(2S,4S)-2-[6-({2-[2-Cyano-4-fluoropyrrolidin-1-yl]-2-oxoethyl}amino)-3-azabicyclo[3.1.0]hex-3-yl]nicotinonitrile (Compound No. 103)
(2S,4S)-4-Fluoro-1-{N-[3-pyrimidin-2-yl-3-azabicyclo[3.1.0]hex-6-yl]glycyl}pyrrolidine-2-carbonitrile (Compound No. 104)
(2S,4S)-4-Fluoro-1-(N-{3-[5-(trifluoromethyl)pyridin-2-yl]-3-azabicyclo[3.1.0]hex-6-yl}glycyl)pyrrolidine-2-carbonitrile (Compound No. 105)
(2S,4S)-1-(N-{3-[(4-Cyanophenyl)sulphonyl]-3-azabicyclo[3.1.0]hex-6-yl}glycyl)-4-fluoropyrrolidine-2-carbonitrile (Compound No. 106)
(2S)-1-{N-[3-(3-Methoxybenzoyl)-3-azabicyclo[3.1.0]hex-6-yl]glycyl}pyrrolidine-2-carbonitrile (Compound No. 107)
(2S)-1-{N-[3-(Biphenyl-4-ylcarbonyl)-3-azabicyclo[3.1.0]hex-6-yl]glycyl}pyrrolidine-2-carbonitrile (Compound No. 108)
3-(4-Fluorobenzoyl)-N-[2-oxo-2-(1,3-thiazolidin-3-yl)ethyl]-3-azabicyclo[3.1.0]hexan-6-amine (Compound No. 109)
6-[6-{[2-Oxo-2-(1,3-thiazolidin-3-yl)ethyl]amino}-3-azabicyclo[3.1.0]hex-3-yl]nicotinonitrile (Compound No. 110)
N-(4-Fluorophenyl)-6-{[2-oxo-2-(1,3-thiazolidin-3-yl)ethyl]amino}-3-azabicyclo[3.1.0]hexane-3-carboxamide (Compound No. 111)
(28)-1-{N-[3-(4-Chlorobenzoyl)-3-azabicyclo[3.1.0]hex-6-yl]gycyl}pyrrolidine-2-carbonitrile (Compound No. 112)
(2S)-1-(N-{3-[(Benzyloxy)acetyl]-3-azabicyclo[3.1.0]hex-6-yl}glycyl)pyrrolidine-2-carbonitrile (Compound No. 113)
(2S)-1-{N-[3-(3,4-Dichlorobenzoyl)-3-azabicyclo[3.1.0]hex-6-yl]glycyl}pyrrolidine-2-carbonitrile (Compound No. 114)
(2S)-1-{N-[3-(2,6-Dimethoxybenzoyl)-3-azabicyclo[3.1.0]hex-6-yl]glycyl}pyrrolidine-2-carbonitrile (Compound No. 115)
(2S)-1-{N-[3-(4-Methoxybenzoyl)-3-azabicyclo[3.1.0]hex-6-yl]glycyl}pyrrolidine-2-carbonitrile (Compound No. 116)
(2S)-1-(N-{3-[(4-Methoxyphenyl)acetyl]-3-azabicyclo[3.1.0]hex-6-yl}glycyl)pyrrolidine-2-carbonitrile (Compound No. 117)
(2S)-1-{N-[3-(4-tert-Butylbenzoyl)-3-azabicyclo[3.1.0]hex-6-yl]glycyl}pyrrolidine-2-carbonitrile (Compound No. 118)
(2S)-1-{N-[3-(2,4-Dichlorobenzoyl)-3-azabicyclo[3.1.0]hex-6-yl]glycyl}pyrrolidine-2-carbonitrile (Compound No. 119)
(2S)-1-{N-[3-(4-Cyanobenzoyl)-3-azabicyclo[3.1.0]hex-6-yl]glycyl}pyrrolidine-2-carbonitrile (Compound No. 120)
(2S)-1-{N-[3-(3-Chlorobenzoyl)-3-azabicyclo[3.1.0]hex-6-yl]glycyl}pyrrolidine-2-carbonitrile (Compound No. 121)
(2S)-1-(N-{3-[4-(Trifluoromethoxy)benzoyl]-3-azabicyclo[3.1.0]hex-6-yl}glycyl)pyrrolidine-2-carbonitrile (Compound No. 122),
(2S)-1-{N-[3-(2,6-Difluorobenzoyl)-3-azabicyclo[3.1.0]hex-6-yl]glycyl}pyrrolidine-2-carbonitrile (Compound No. 123),
(2S)-1-{N-[3-(4-Methylbenzoyl)-3-azabicyclo[3.1.0]hex-6-yl]glycyl}pyrrolidine-2-carbonitrile (Compound No. 124),
(2S,4S)—N-{4-[6-({2-[2-Cyano-4-fluoropyrrolidin-1-yl]-2-oxoethyl}amino)-3-azabicyclo[3.1.0]hex-3-yl]phenyl}methanesulphonamide (Compound No. 125)
(2S,4S)—N-{4-[6-({2-[2-Cyano-4-fluoropyrrolidin-1-yl]-2-oxoethyl}amino)-3-azabicyclo[3.1.0]hex-3-yl]phenyl}-4-methylbenzenesulphonamide (Compound No. 126), or
(2S,4S, 5S)-1-{N-[3-(4-Methylbenzoyl)-3-azabicyclo[3.1.0]hex-6-yl]glycyl}-4,5-methanopyrrolidine-2-carbonitrile (Compound No. 127).

16. A pharmaceutical composition comprising a therapeutically effective amount of a 2 compound as defined in claim 1 together with a pharmaceutically acceptable 3 carrier, excipients or diluents.

17. A pharmaceutical composition of claim 6 further comprising one or more additional active ingredients selected from:
1) antihyperglycaemic agents (a) other DPP IV inhibitors, (b) insulin sensitizers, (I) PPAR agonists, (II) PPARα/γ dual agonists, and (III) PPAR pan-agonists, (c) biguanides, (d) insulin secretagogues, (e) α-glucosidase inhibitors, (f) protein tyrosine phosphatase-1B inhibitors, (g) glucokinase activators, (h) inhibitors of 11β-hydroxysteroid dehydrogenase type 1, (i) glucagon receptor antagonists, (j) GLP-1 and GLP-1 receptor agonists, (k) insulin or insulin mimetics, (l) GIP and GIP receptor agonists (m) PACAP and PACAP receptor agonists, (n) fructose 1,6 bisphosphatase inhibitors; (o) glucose 6 phosphatase inhibitors; (p) Sodium glucose transporter 2 (SGLT2) inhibitor, (q) AMP-Activated protein kinase activators;
2) lipid modulating agents, (i) HMG-CoA reductase inhibitors, (ii) sequestrants (iii) nicotinyl alcohol, nicotinic acid or a salt thereof, (iv) inhibitors of cholesterol absorption, (v) acyl CoA:cholesterol acyltransferase inhibitors, (vi) ileal bile acid transporter inhibitors, and (vi) CETP inhibitors;
3) antiobesity compounds, (i) CB1 receptor inverse agonists and antagonists, (ii) β₃ adrenergic receptor agonists, (iii) melanocortin-receptor agonists, (iv) ghrelin antagonists, (v) neuropeptide Y₁ or Y₅ antagonists, (vi) melanin-concentrating hormone (MCH) receptor antagonists and (vii) fenfluramine, dexfenfluramine, phentermine, sibutramine, or orlistat;
4) antihypertensive agents, (i) ACE inhibitors, (ii) angiotensin II receptor antagonists and agonists, (iii) β-blockers, and (iv) calcium channel blockers; and
5) anti-TNF agent or c-AMP raising agent.

8. A method for treating conditions mediated by DPP IV, like diabetes, especially type 2 diabetes, as well as prediabetes, diabetic dyslipidemia, metabolic acidosis, ketosis, satiety disorders, and obesity, various conditions manifested by a variety of metabolic, neurological, anti-inflammatory, and autoimmune disorders like inflammatory disease, multiple sclerosis, rheumatoid arthritis; viral, cancer and gastrointestinal disorders and treatment of infertility arising due to polycystic ovary syndrome, which comprises administering to the mammal an effective amount of a compound of claim 1.

9. A method for treating conditions mediated by DPP IV, like diabetes, especially type 2 diabetes, as well as prediabetes, diabetic dyslipidemia, metabolic acidosis, ketosis, satiety disorders, and obesity, various conditions manifested by a variety of metabolic, neurological, anti-inflammatory, and autoimmune disorders like inflammatory disease, multiple sclerosis, rheumatoid arthritis; viral, cancer and gastrointestinal disorders and treatment of infertility arising due to polycystic ovary syndrome, which comprises administering to the mammal an effective amount of a pharmaceutical composition according to claim 6.

10. A method for treating conditions mediated by DPP IV, like diabetes, especially type 2 diabetes, as well as prediabetes, diabetic dyslipidemia, metabolic acidosis, ketosis, satiety disorders, and obesity, various conditions manifested by a variety of metabolic, neurological, anti-inflammatory, and autoimmune disorders like inflammatory disease, multiple sclerosis, rheumatoid arthritis; viral, cancer and gastrointestinal disorders and treatment of infertility arising due to polycystic ovary syndrome, which comprises administering to the mammal an effective amount of pharmaceutical composition according to claim 7.

11. A process for preparing a compound of Formula VI, comprising:
a) Reaction of a compound of Formula II 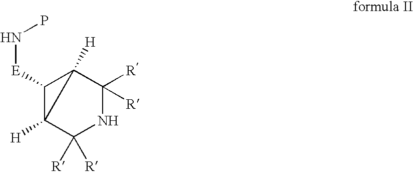
with a compound of Formula III
Z-Y—X-L formula III
to give a compound or Formula Va 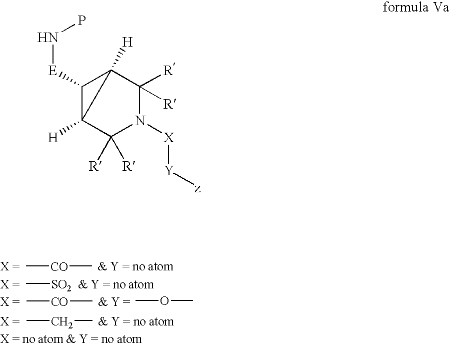
b) The deprotection of a compound of Formula Va to give a compound of Formula VI 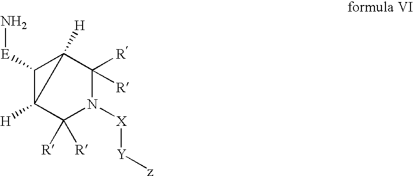
wherein
E is —(CRₓR_{y})ₘ— or —C(RₓR_{y})CON(Rₓ)— [wherein m is an integer of 0 to 3 and Rₓ and R_{y} are independently selected from the group consisting of hydrogen, alkyl, alkenyl, alkynyl, cycloalkyl, aryl, heteroaryl or heterocyclyl];
X is no atom, —CO—, —CS—, —C(RₓR_{y})₁— and —SO₂—, wherein 1 is an integer of 1-3; and Rₓ and R_{y} are defined as above;
Y is no atom, —O— or —NRₓ— with the proviso that Y cannot be —O— or —NRₓ— when X═C(RₓR_{y})₁ [wherein 1 is 1];
Z is alkyl, alkenyl, alkynyl, cycloalkyl, aryl, heteroaryl or heterocyclyl;
R′ is Rₓ, —CN, carboxy, halogen, carbonyl, or amino; and
P is an amino protecting group selected from Boc, Fmoc, allyloxycarbonyl or benzyl derivative.

12. A process for preparing a compound of Formula VI, comprising:
a) reaction of a compound of Formula II with a compound of Formula IV
Z-N═C=M
M=O,S formula IV
to give a compound Formula Vb 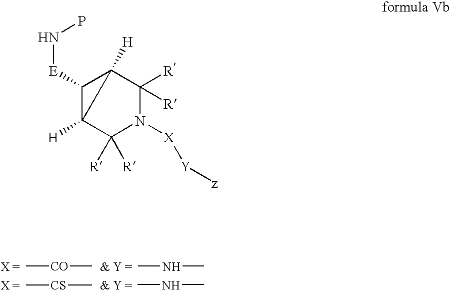
b) the deprotection of a compound of Formula Vb to give a compound of Formula VI
wherein
E is —(CRₓR_{y})ₘ— or —C(RₓR_{y})CON(Rₓ)— [wherein m is an integer of 0 to 3 and Rₓ and R_{y} are independently selected from the group consisting of hydrogen, alkyl, alkenyl, alkynyl, cycloalkyl, aryl, heteroaryl or heterocyclyl];
X is no atom, —CO—, —CS—, —C(RₓR_{y})₁— and —SO₂—, wherein 1 is an integer of 1-3; and Rₓ and R_{y} are defined as above;
Y is no atom, —O— or —NRₓ— with the proviso that Y cannot be —O— or —NRₓ— when X═C(RₓR_{y})₁ [wherein 1 is 1];
Z is alkyl, alkenyl, alkynyl, cycloalkyl, aryl, heteroaryl or heterocyclyl;
R′ is Rₓ, —CN, carboxy, halogen, carbonyl, or amino; and
P is an amino protecting group selected from Boc, Fmoc, allyloxycarbonyl or benzyl derivative.

13. A process for preparing a compound of Formula VI, comprising:
a) reaction of a compound of Formula VI 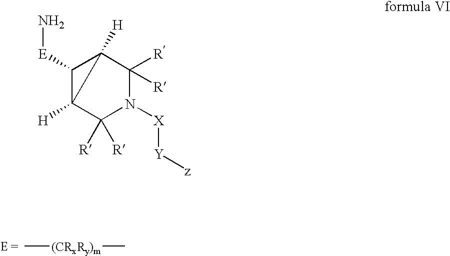
with a compound of Formula VII 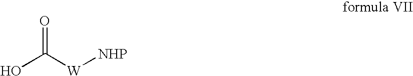
to give a compound Formula VIII 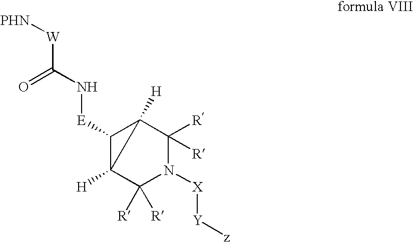
b) the deprotection of a compound of Formula VIII to give a compound of Formula IX 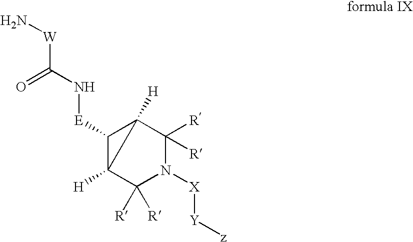
wherein
W is —C(RₓR_{y})ₙ— wherein n is an integer of 1 to 3 and Rₓ and R_{y} are independently selected from the group consisting of hydrogen, alkyl, alkenyl, alkynyl, cycloalkyl, aryl, heteroaryl or heterocyclyl;
E is —(CRₓR_{y})ₘ— [wherein m is an integer of 0 to 3];
X is no atom, —CO—, —CS—, —C(RₓR_{y})ₙ— and —SO₂—, wherein 1 is an integer of 1-3; and Rₓ and R_{y} are defined as above;
Y is no atom, —O— or —Nₓ— with the proviso that Y cannot be —O— or —NRₓ— when X═C(RₓR_{y})₁ [wherein 1 is 1];
Z is alkyl, alkenyl, alkynyl, cycloalkyl, aryl, heteroaryl or heterocyclyl;
R′ is Rₓ, —CN, carboxy, halogen, carbonyl, or amino: and
P is an amino protecting group selected from Boc, Fmoc, allyloxycarbonyl or benzyl derivative.

14. A process for preparing the compounds of Formula XI and Formula XII, comprising:
a) reaction of a compound of Formula X 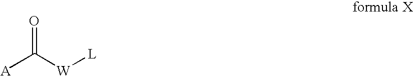
with a compound of Formula VI 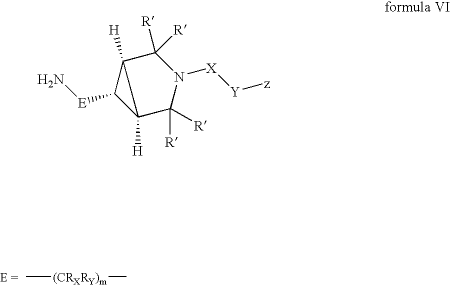
to give a compound Formula XI 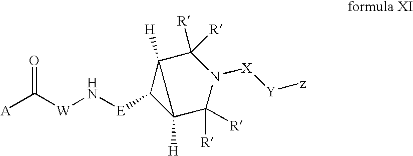
and
b) reaction of a compound of Formula X with a compound of Formula IX 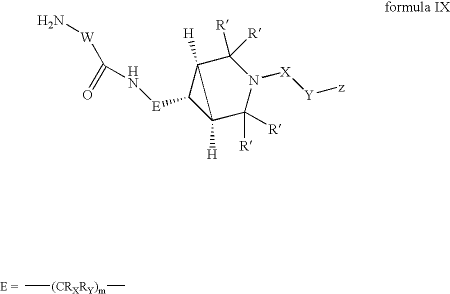
to give a compound Formula XII 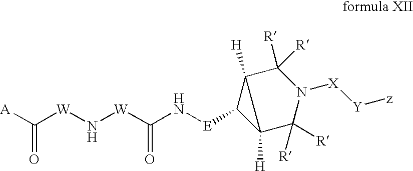
wherein
A is a group selected from 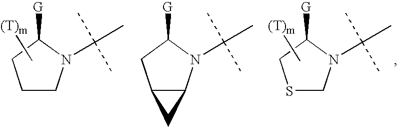
wherein
G is selected from H, —CN, —COR₁, —CR₂═NOH, —CR₂═NR₂ or B(R₃)(R₄) (wherein R₁ is hydrogen, CF₃, alkyl, aryl or heteroaryl;
R₂ is H, alkyl, aryl or heteroaryl; R₃ and R₄ are independently selected from —OH or —OR₅ [wherein —OR₅ can be hydrolysed to —OH and R₅ is alkyl, cycloalkyl or aryl]; If R₃ and R₄ are OR₅, then R₃ and R₄ may together form a ring of 5 to 8 atoms);
T is cyano, halogen, alkyl, alkenyl, alkynyl, hydroxy, alkoxy, carbonyl, thiocarbonyl, and oxo.
W is —C(RₓR_{y})_{d}— wherein n is an integer of 1 to 3 and Rₓ and R_{y} are independently selected from the group consisting of hydrogen, alkyl, alkenyl, alkynyl, cycloalkyl, aryl, heteroaryl or heterocyclyl;
E is —(CRₓR_{y})ₘ— or —C(RₓR_{y})CON(Rₓ)— [wherein m is an integer of 0 to 3]
X is no atom, —CO—, —CS—, —C(RₓR_{y})ₙ— and —SO₂—, wherein 1 is an integer of 1-3; and Rₓ and R_{y} are defined as above;
Y is no atom, —O— or —NRₓ— with the proviso that Y cannot be —O— or —NRₓ— when X═C(RₓR_{y})₁ [wherein 1 is 1];
Z is alkyl, alkenyl, alkynyl, cycloalkyl, aryl, heteroaryl or heterocyclyl;
R′ is Rₓ, —CN, carboxy, halogen, carbonyl, or amino; and
L is a leaving group such as halide.
